(19) United States
(12) Patent Application Publication
(10) Pub. No.: US 2020/0054353 A1
(43) Pub. Date: Feb. 20, 2020

(54) MAXILLARY SINUS PUNCTURE APPARATUS

(71) Applicants: Yeong Seok YUN, Incheon (KR); Seong Ho YUN, Incheon (KR); Seong Jin YUN, Incheon (KR)

(72) Inventors: Yeong Seok YUN, Incheon (KR); Seong Ho YUN, Incheon (KR); Seong Jin YUN, Incheon (KR)

(21) Appl. No.: 16/491,351

(22) PCT Filed: May 8, 2017
(86) PCT No.: PCT/KR2017/004768

§ 371 Date: Sep. 5, 2019

(30) Foreign Application Priority Data
Mar. 15, 2017 (KR) 10-2017-0032409

(51) Int. Cl. A61B 17/24; A61B 17/34; A61B 1/00; A61B 1/233; A61B 18/14; A61B 1/05; A61B 1/06; A61B 8/12; A61B 90/00

(52) U.S. Cl. CPC A61B 17/24; A61B 17/3403; A61B 1/00135; A61B 1/233; A61B 18/1485; A61B 2018/00327; A61B 1/0684; A61B 8/12; A61B 90/39; A61B 2217/007; A61B 1/05

(57) ABSTRACT

The present invention relates to an apparatus for puncturing a maxillary sinus, the apparatus including: a main body gripped by an operator; a probing unit detachable from the main body and configured to detect a posterior fontanelle; a surgical procedure tube which is provided in the main body and formed of an endoscope unit configured to monitor the posterior fontanelle, a puncturing unit configured to puncture the posterior fontanelle, and an irrigation tube configured to irrigate the maxillary sinus through a punctured portion of the posterior fontanelle; and an operation unit provided in the main body and configured to operate the surgical procedure tube.

### TECHNICAL FIELD

The present invention relates to an apparatus for puncturing a maxillary sinus.

### BACKGROUND ART

Small cavities exist within a human's head, and the cavities are called sinuses and are connected to a nose through thin ducts, etc. Here, the sinuses include a maxillary sinus, an ethmoid sinus, a frontal sinus, a sphenoid sinus, etc.

A phenomenon in which sinuses are inflamed is called paranasal sinusitis, which is commonly referred to as sinusitis. Particularly, inflammation of a maxillary sinus is called maxillary sinusitis. Medication and surgery have been widely used to treat maxillary sinusitis.

However, when maxillary sinusitis does not respond well to medication, the maxillary sinus is punctured, an irrigation tube is injected into the maxillary sinus, the maxillary sinus is irrigated through the irrigation tube, and drugs such as antibiotics are injected into the maxillary sinus.

In this case, the use of antibiotics through oral administration or injection may be decreased, a treatment period may be shortened, maxillary sinusitis may be prevented from being aggravated to a level requiring surgery, and even patients facing difficulty using oral antibiotics due to gastrointestinal problems, etc. may be effectively treated.

Antibiotic tolerance increasing due to long-term administration of antibiotics is being recognized as a social problem. Accordingly, antibiotics locally acting only on sinuses need to be used instead of systemically-acting antibiotics, i.e., those that are orally administered or injected. Treatment using a drug delivery system, in which anticancer drugs, steroids, or antibiotics are bonded to small particles to be injected into the body, has recently gained attention. The injected drugs are gradually secreted over a month to three months and become effective. Also, the small particles are gradually decomposed and eventually disappear. The small particles are referred to as drug eluting beads. Drug eluting beads have various sizes ranging from 100 to 700 μm, and because they are formed of materials such as albumin, etc., they are safe to use in the human body and will gradually break down and disappear. When a predetermined amount of time passes after drugs are mixed with drug-eluting beads, the drugs are absorbed into the drug-eluting beads. In addition to antibiotics, drugs used for sinusitis or rhinitis may also be mixed together with drug-eluting beads. Then, an effect of taking drugs for a long period of time may be achieved by one surgical procedure, i.e., injecting the drug-eluting beads containing the drugs into the maxillary sinus. Also, even when drugs need to be taken for a long period of time, a desired effect may be achieved by acting on sinuses or nasal cavities while having almost no systemic influence, thereby significantly decreasing tolerance to drugs or possibilities of side effects.

In addition to treatment purposes, the maxillary sinus may be punctured to suction out a substance within the maxillary sinus for use in bacteriological tests or other research activities. Here, inflammation caused by anaerobic bacteria may be prevented when an irrigation tube is left within the maxillary sinus instead of being removed since oxygen is continuously supplied to the maxillary sinus through the irrigation tube.

Conventional methods for puncturing/irrigating a maxillary sinus include a method of puncturing a maxillary sinus through an inferior meatus, a method of puncturing a maxillary sinus through a canine fossa, the Proetz irrigation method and an irrigation method through a maxillary sinus ostium.

The puncturing method using the inferior meatus involves puncturing from the inferior meatus toward the maxillary sinus. A probability of failure is high when bones near a puncture are thick, a mucosal injury is a concern, and a needle for puncturing may injure eyes.

In addition, the puncturing method using the canine fossa involves puncturing toward the maxillary sinus through the oral cavity. This method has a difficulty of having to pass through the oral cavity and a concern of injuring bones at the oral mucosa and canine fossa.

In addition, in the Proetz irrigation method, a liquid medicine is injected into nasal cavities while a patient's head is leaned backward and inner portions of the nasal cavities are converted with a negative pressure to enable the liquid medicine to flow into the maxillary sinus. In this case, there is a problem of causing discomfort for the patient during the surgical procedure.

In addition, the irrigation method through a maxillary sinus ostium is a method of injecting an irrigation tube through the maxillary sinus ostium which is an inlet from the maxillary sinus toward the middle meatus. However, there is a problem of an extreme difficulty in injecting the irrigation tube through the maxillary sinus ostium since the maxillary sinus ostium is occluded by bones, etc.

Since the conventionally known methods for puncturing/irrigating a maxillary sinus have so far failed to properly address discomfort felt by a patient and a doctor performing the surgical procedure, dangers of the surgical procedure, etc., research and development are needed.

### DISCLOSURE

### Technical Problem

The present invention has been devised to solve the above problems of the related art, and the present invention is directed to providing a maxillary sinus puncturing apparatus that punctures a posterior fontanelle located in the middle meatus, thereby securing safety due to not puncturing bones and facilitating the puncturing of the maxillary sinus.

In addition, the present invention is directed to providing a maxillary sinus puncturing apparatus that detects a posterior fontanelle where there are no bones using ultrasonic probing, punctures the detected posterior fontanelle, and allows an irrigation tube to be installed, thereby effectively and quickly treating maxillary sinusitis.

### Technical Solution

According to an aspect of the present invention, a maxillary sinus puncturing apparatus includes: a main body gripped by an operator; a probing unit detachable from the main body and configured to detect a posterior fontanelle; a surgical procedure tube which is provided in the main body and formed of an endoscope unit configured to monitor the posterior fontanelle, a puncturing unit configured to puncture the posterior fontanelle, and an irrigation tube configured to irrigate a maxillary sinus through a punctured portion of the posterior fontanelle; and an operation unit provided in the main body and configured to operate the surgical procedure tube.

Specifically, the surgical procedure tube may include a sheath configured to accommodate the puncturing unit, the irrigation tube, and the endoscope unit, and the sheath may include a guide structure which forms a front end of the sheath and has a convex outer shape and a round inner shape; a sheath space which provides a place where a front end of the puncturing unit is bendable and in which a space region is secured by the guide structure; a sheath opening configured to provide a passage through which the front end of the puncturing unit guided by the guide structure passes; a surgical procedure means insertion passage through which the puncturing unit and the irrigation tube are inserted and mounted; and an endoscope insertion passage through which the endoscope unit is inserted and mounted.

Specifically, the sheath may further include a direction-changing protrusion which is provided at an upper portion of an inner peripheral surface of the surgical procedure means insertion passage and configured to guide the front end of the puncturing unit to be bent toward the sheath opening before coming into contact with a round surface of the guide structure when the puncturing unit moves forward in order to puncture the posterior fontanelle.

Specifically, the sheath may further include an emergency treatment hole which is provided at an upper portion of the guide structure so as to pass through the sheath space and configured to allow emergency treatment to be performed when the puncturing unit or the irrigation tube is locked to an inner wall of the guide structure.

Specifically, the guide structure may be configured as a fixed type or a movable type.

Specifically, the movable guide structure may be configured so that a front portion is thick as a whole, a middle portion is thin as a whole, and a rear portion is thin as the middle portion but has a lower end which is thick as the front portion.

Specifically, a fixing key configured to restrict a forward movement may be further provided in the movable guide structure.

Specifically, the puncturing unit may include: a puncturing needle which is provided in the surgical procedure means insertion passage, forms the front end of the puncturing unit, and is configured to cauterize the posterior fontanelle using electricity and puncture the posterior fontanelle; a cautery wire which is provided in the surgical procedure means insertion passage, is moved by the operation unit, and passes through the surgical procedure means insertion passage so as to supply electricity to the puncturing needle; and a puncturing electrocauterizer main body which is provided in the main body and configured to supply electricity to the puncturing needle through the cautery wire.

Specifically, the puncturing unit may further include: a cautery activation button which is provided in the main body and configured to activate a cauterizing function of the puncturing electrocauterizer main body; a switch which is provided in the main body and configured to turn on or off the electricity for driving the puncturing electrocauterizer main body; and a socket which is provided in the main body and provided between the puncturing electrocauterizer main body and the cautery wire.

Specifically, the irrigation tube may be provided to be fitted to the cautery wire and be installed in the posterior fontanelle punctured by the puncturing needle.

Specifically, a locking protrusion configured to prevent the irrigation tube from moving backward when the cautery wire moves forward may be further provided, a groove with an open upper portion may be formed in the locking protrusion, a towline may be further provided at an inlet, through which drugs are injected, of the irrigation tube, and, in a state in which the irrigation tube is inserted into the maxillary sinus, the towline may move the irrigation tube upward and forward through the groove of the locking protrusion so that the irrigation tube is fixed so as not to be pushed backward.

Specifically, the surgical procedure means insertion passage may extend to a predetermined length forward from a front end of the main body and be formed of a portion inserted into nasal cavities during a surgical procedure and a portion provided at an upper portion of the main body and exposed to the outside during the surgical procedure, a passage diameter of the portion exposed to the outside may be larger than a passage diameter of the portion inserted into the nasal cavities, and an inlet portion may be provided at a rear end of the portion exposed to the outside.

Specifically, the puncturing unit may further include a cautery wire protection tube which has one end fixed to the operation unit and extends to a boundary between the portion inserted into the nasal cavities and the portion exposed to the outside, and the cautery wire protection tube may be inserted and mounted on the portion exposed to the outside and be fixed by a fixing clip provided at the inlet portion.

Specifically, the endoscope unit may include: an endoscope camera main body provided in the main body; an endoscope camera monitor rotatably mounted on the endoscope camera main body; an endoscope camera head provided in the main body and connected to the endoscope camera main body; an endoscope head provided in the main body and connected to the endoscope camera head; an endoscope mounted on an inner portion of the endoscope insertion passage and connected to the endoscope head; an endoscope window connected to a front end of the endoscope and provided to be exposed to the outside at a front end of the endoscope insertion passage so as to secure a monitoring field-of-view; and a small light-emitting diode (LED) light source device or light source cable configured to transmit light to the endoscope.

Specifically, the endoscope camera main body may be provided at a rear end of the main body, have a battery embedded therein, and be manufactured so that an upper end surface thereof is placed on a straight line with an upper end surface of the main body.

Specifically, the probing unit may be an ultrasonic probing unit configured to detect a punctured position of the posterior fontanelle using ultrasonic waves, and the ultrasonic probing unit may include: an ultrasonic main body provided at a rear end of a probing tube; an ultrasonic probe which is provided at a front end of the probing tube and has one side surface at which a liquid structure is provided; and a coaxial lead wire provided inside the probing tube and configured to transmit an electrical signal from the ultrasonic main body to the ultrasonic probe.

Specifically, the ultrasonic probing unit may further include: a probing unit handle provided at a front portion of the ultrasonic main body; and a probing monitor provided at a rear portion of the ultrasonic main body.

Specifically, the maxillary sinus puncturing apparatus may further include a marking unit configured to mark a position to be punctured on a mucosa of the posterior fontanelle, and the marking unit may include: a marking electrocauterizer main body provided between the ultrasonic main body and the probing tube; a marking activation button configured to activate a marking function of the marking electrocauterizer main body; a needle passage which is provided in the ultrasonic probe and has a structure open toward the liquid structure; a wire which is provided inside the probing tube and extends from the marking electrocauterizer main body to the needle passage; and a marking needle which is provided inside the needle passage and connected to the wire so as to receive electricity from the marking electrocauterizer main body, cauterize the mucosa of the posterior fontanelle, and mark the position to be punctured thereon.

Specifically, the maxillary sinus puncturing apparatus may further include a marking unit configured to mark a position to be punctured on a mucosa of the posterior fontanelle, and the marking unit may include: a cylinder tube which is provided in the ultrasonic probe and has a structure open toward the liquid structure; a fluid pipe which is provided in a longitudinal direction inside the probing tube and extends to the cylinder tube by passing through the ultrasonic probe; a push-pull means which is connected to one end of the fluid pipe at an inner upper portion of the cylinder tube and configured to expand or contract due to a fluid injected or discharged through the fluid pipe; a piston which is connected to the push-pull means at an inner lower portion of the cylinder tube and configured to move upward and downward due to an operation of the push-pull means; and a marking needle which is connected to the piston at the inner lower portion of the cylinder tube and configured to, according to upward and downward movement of the piston, leave a scratch on the mucosa of the posterior fontanelle to mark a position to be punctured thereon.

Specifically, a plurality of guide bars may be provided at an inner lower surface of the cylinder tube to allow the piston to move upward and downward uniformly, and a plurality of guide grooves corresponding to the plurality of guide bars may be formed in the piston.

Specifically, the maxillary sinus puncturing apparatus may further include a marking unit configured to mark a position to be punctured on a mucosa of the posterior fontanelle, and the marking unit may include: a cylinder tube which is provided in the ultrasonic probe and has a structure open toward the liquid structure; a fluid pipe which is provided in a longitudinal direction outside the probing tube and extends to the cylinder tube by passing through the ultrasonic probe; a piston which is accommodated in the cylinder tube and configured to move upward and downward due to a fluid injected or discharged through the fluid pipe; a wire which is provided inside the fluid pipe and extends from an electricity supply source to the cylinder tube; an airtight member which has one end connected to the cylinder tube and the other end connected to the piston and is configured to prevent the fluid injected into the fluid pipe from leaking to the outside; and a tip which is provided at a lower end of the piston and configured to mark a position to be punctured on the mucosa of the posterior fontanelle using an electrocauterization method.

Specifically, the tip may mark the position to be punctured on the mucosa of the posterior fontanelle and then puncture the posterior fontanelle using the electrocauterization method.

Specifically, the probing unit may be a light probing unit configured to detect a punctured position of the posterior fontanelle using light, and the light probing unit may include: a light probing main body; a light emitting unit formed of a first probing tube, which is connected to the light probing main body, and a first light probe, which is provided at a front end of the first probing tube and configured to receive an electrical signal from the light probing main body and emit light; and a light receiving unit formed of a second probing tube, which is connected to the light probing main body, and a second light probe, which is provided at a front end of the second probing tube and configured to sense light emitted from the first light probe and transmit the light to the light probing main body.

According to an aspect of the present invention, a maxillary sinus puncturing apparatus includes: a main body gripped by an operator; a surgical procedure tube provided in the main body; and an operation unit configured to operate the surgical procedure tube, wherein the surgical procedure tube includes a probing tube configured to allow a posterior fontanelle to be detected by an ultrasonic probing unit, and a marking/puncturing tube configured to allow the maxillary sinus to be punctured after a position to be punctured is marked on a mucosa of the posterior fontanelle by a marking/puncturing unit.

Specifically, the surgical procedure tube may further include a liquid tube configured to allow a liquid to be injected into or discharged from a liquid structure provided at one side surface of an ultrasonic probe of the ultrasonic probing unit.

Specifically, each of the probing tube, the marking/puncturing tube, and the liquid tube may be provided in a lengthy form to be inserted into a middle meatus and may be disposed to be parallel to one another.

Specifically, the liquid tube may have a structure connected from the ultrasonic probe to the main body, an outlet portion of the liquid tube may be connected to the liquid structure, and an inlet portion of the liquid tube may be connected to a liquid injection/discharge means provided at a portion of the main body.

Specifically, the liquid injection/discharge means may include: a valve provided inside the inlet portion of the liquid tube; and a liquid supply unit detachable from the inlet portion of the liquid tube.

Specifically, the ultrasonic probing unit may include: an ultrasonic main body which is provided at a rear end of the probing tube at a rear portion of the main body and configured to generate an electrical signal for generating ultrasonic waves; an ultrasonic probe which is provided at a front end of the probing tube and configured to detect the posterior fontanelle; the liquid structure provided at one side surface of the ultrasonic probe; a coaxial lead wire which is provided inside the probing tube to pass therethrough and configured to transmit an electrical signal from the ultrasonic main body to the ultrasonic probe; and a probing monitor provided at a rear portion of the ultrasonic main body.

Specifically, the probing tube may have a structure connected from the ultrasonic probe to the rear portion of the main body, the ultrasonic probe may be connected to a front end of the probing tube, the ultrasonic main body may be connected to a rear end of the probing tube, and the coaxial lead wire configured to transmit an electrical signal to the ultrasonic probe may be disposed inside the probing tube.

Specifically, the marking/puncturing unit may include: a marking/puncturing needle which is disposed at an outlet portion of the marking/puncturing tube and configured to cauterize the posterior fontanelle, mark a position to be punctured on a mucosa of the posterior fontanelle, and then puncture the maxillary sinus; a cautery wire which is provided inside the marking/puncturing tube and moved by the operation unit and is configured to pass through the marking/puncturing tube to supply electricity to the marking/puncturing needle; and a marking/puncturing electrocauterizer main body which is provided in the main body and configured to supply electricity to the marking/puncturing needle through the cautery wire.

Specifically, the marking/puncturing unit may further include: a cautery activation switch which is provided in the main body and configured to activate a cauterizing function of the marking/puncturing electrocauterizer main body; and a main body socket which is provided in the main body and provided between the marking/puncturing electrocauterizer main body and the cautery wire.

Specifically, the marking/puncturing unit may further include an emergency treatment hole which is formed at the other side surface of the ultrasonic probe, which is a surface opposite to the liquid structure provided at the one side surface of the ultrasonic probe of the ultrasonic probing unit, and formed to pass through the outlet portion of the marking/puncturing tube from the other side surface of the ultrasonic probe.

Specifically, the marking/puncturing tube may have a structure connected from the ultrasonic probe to the main body, the outlet portion through which the marking/puncturing needle moves in and out of the marking/puncturing tube may be formed at a portion of the ultrasonic probe, an inlet portion through which the marking/puncturing needle is inserted into the marking/puncturing tube may be formed at a portion of the main body, and the cautery wire configured to transmit electricity to the marking/puncturing needle may be disposed inside the marking/puncturing tube.

### Advantageous Effects

The maxillary sinus puncturing apparatus according to the present invention punctures a posterior fontanelle in order to install an irrigation tube in the maxillary sinus such that the maxillary sinus may be punctured without puncturing bones, thereby guaranteeing a safe surgical procedure.

In addition, the maxillary sinus puncturing apparatus according to the present invention probes a posterior fontanelle where there are no bones using ultrasonic waves in order to puncture the maxillary sinus such that convenience is improved and a treatment time is significantly shortened, thereby improving satisfaction of a patient and a doctor.

### DESCRIPTION OF DRAWINGS

FIG. 1 is a side view of a maxillary sinus puncturing apparatus according to a first embodiment of the present invention.

FIG. 2 is a plan view of the maxillary sinus puncturing apparatus according to the first embodiment of the present invention.

FIGS. 3 and 4 are cross-sectional views taken along line A-A′ of FIG. 1.

FIG. 5 is a plan view of a puncturing tube in the maxillary sinus puncturing apparatus according to the first embodiment of the present invention.

FIGS. 6A and 6B are a side cross-sectional view and a plan cross-sectional view, respectively, that illustrate another embodiment of a puncturing tube in the maxillary sinus puncturing apparatus according to the first embodiment of the present invention.

FIGS. 7A to 7C are sequence diagrams for describing a method of operating the puncturing tube of FIG. 6.

FIGS. 8A and 8B are a side view and a plan view, respectively, that illustrate another embodiment of an ultrasonic probe in the maxillary sinus puncturing apparatus according to the first embodiment of the present invention.

FIG. 9 is a view illustrating a cautery wire protection tube in the maxillary sinus puncturing apparatus according to the first embodiment of the present invention.

FIG. 10 is a view illustrating a moving block position adjuster in the maxillary sinus puncturing apparatus according to the first embodiment of the present invention.

FIGS. 11A to 11C are views illustrating a method of detecting a posterior fontanelle by the maxillary sinus puncturing apparatus according to the first embodiment of the present invention.

FIGS. 12A and 12B are views illustrating a result detected in accordance with the method of detecting a posterior fontanelle of FIG. 11.

FIG. 13 is an exploded side view of the maxillary sinus puncturing apparatus according to the first embodiment of the present invention.

FIGS. 14A and 14B are a side view and a plan view, respectively, that illustrate another embodiment of an ultrasonic probing unit in the maxillary sinus puncturing apparatus according to the first embodiment of the present invention.

FIGS. 15A and 15B are a side view and a plan view, respectively, of the ultrasonic probing unit to which a probing unit cover is added.

FIG. 16 is a view illustrating still another embodiment of an ultrasonic probing unit in the maxillary sinus puncturing apparatus according to the first embodiment of the present invention.

FIG. 17 is a view for describing an image shown on a probing monitor of the ultrasonic probing unit.

FIG. 18 is a side view illustrating another embodiment of a moving unit in the maxillary sinus puncturing apparatus according to the first embodiment of the present invention.

FIG. 19 is a cross-sectional view taken along line A-A′ of FIG. 18.

FIG. 20 is a view for describing a maxillary sinus tube.

FIG. 21 is a view for describing a tube front wing of the maxillary sinus tube.

FIG. 22 is a view for describing inserting and installing the maxillary sinus tube at a punctured portion of the posterior fontanelle using a tube insertion tool.

FIG. 23 is a side cross-sectional view of a face.

FIG. 24 is a front cross-sectional view of the face.

FIG. 25 is an exploded view for describing a maxillary sinus puncturing apparatus according to a second embodiment of the present invention.

FIGS. 26A to 26C are views for describing an embodiment of a surgical procedure tube in the maxillary sinus puncturing apparatus according to the second embodiment of the present invention.

FIGS. 27A to 27C are views for describing another embodiment of a surgical procedure tube in the maxillary sinus puncturing apparatus according to the second embodiment of the present invention.

FIGS. 28A to 28C are views for describing still another embodiment of a surgical procedure tube in the maxillary sinus puncturing apparatus according to the second embodiment of the present invention.

FIGS. 29A to 29C are views for describing the shape of a movable guide structure of FIGS. 27 and 28.

FIG. 30 is a view for describing the shape of an endoscope window of FIGS. 27 and 28.

FIG. 31 is a front cross-sectional view of a face for describing the fact that the surgical procedure tube is disposed in the posterior fontanelle.

FIG. 32 is an enlarged view of portions of a cautery wire protection tube and a moving unit in the maxillary sinus puncturing apparatus according to the second embodiment of the present invention.

FIG. 33 is an enlarged view of a portion of a trigger in the maxillary sinus puncturing apparatus according to the second embodiment of the present invention.

FIG. 34 is a cross-sectional view taken along line Y-Y′ of FIG. 33.

FIG. 35 is a view illustrating an ultrasonic probing unit coupled to a marking unit in the maxillary sinus puncturing apparatus according to the second embodiment of the present invention.

FIG. 36 is a cross-sectional view taken along line X-X′ of FIG. 35 that is for describing a first embodiment of the marking unit in the maxillary sinus puncturing apparatus according to the second embodiment of the present invention.

FIG. 37 is a cross-sectional view taken along line X-X′ of FIG. 35 that is for describing a second embodiment of the marking unit in the maxillary sinus puncturing apparatus according to the second embodiment of the present invention.

FIG. 38 is a view for describing a coupling structure between a piston and a cylinder tube in FIG. 37.

FIGS. 39A to 39C are cross-sectional views taken along line X-X′ of FIG. 35 that are for describing a third embodiment of the marking unit in the maxillary sinus puncturing apparatus according to the second embodiment of the present invention.

FIG. 40 is a cross-sectional view taken along line X-X′ of FIG. 35 that is for describing a fourth embodiment of the marking unit in the maxillary sinus puncturing apparatus according to the second embodiment of the present invention.

FIGS. 41A to 41C are views for describing an irrigation tube.

FIG. 42 is a front cross-sectional view of a face illustrating a state in which the irrigation tube is inserted into the maxillary sinus.

FIG. 43 is a view illustrating a light probing unit configured to detect a posterior fontanelle using light in the maxillary sinus puncturing apparatus according to the present invention.

FIG. 44 is a left cross-sectional view of a face for describing a method of detecting a posterior fontanelle using the light probing unit of FIG. 43.

FIG. 45 is a bottom view of a maxillary sinus puncturing apparatus according to a third embodiment of the present invention.

FIG. 46 is a side view of the maxillary sinus puncturing apparatus according to the third embodiment of the present invention.

FIGS. 47A to 47C are enlarged views of portion A of FIG. 45.

FIG. 48 is an enlarged view of portion B of FIG. 46.

FIGS. 49A to 49D are views for describing a cautery activation switch.

### MODES OF THE INVENTION

The objectives, specific advantages, and novel features of the present invention will become more apparent from the detailed description and the preferred embodiments below to be referenced to the accompanying drawings. In giving reference numerals to elements in each drawing of this specification, it should be noted that like reference numerals are given to like elements as much as possible even when the elements are illustrated in different drawings. In addition, in describing the present invention, when detailed description on a known related art is deemed to unnecessarily obscure the gist of the present invention, the detailed description thereof will be omitted.

Hereinafter, the preferred embodiments of the present invention will be described in detail with reference to the accompanying drawings.

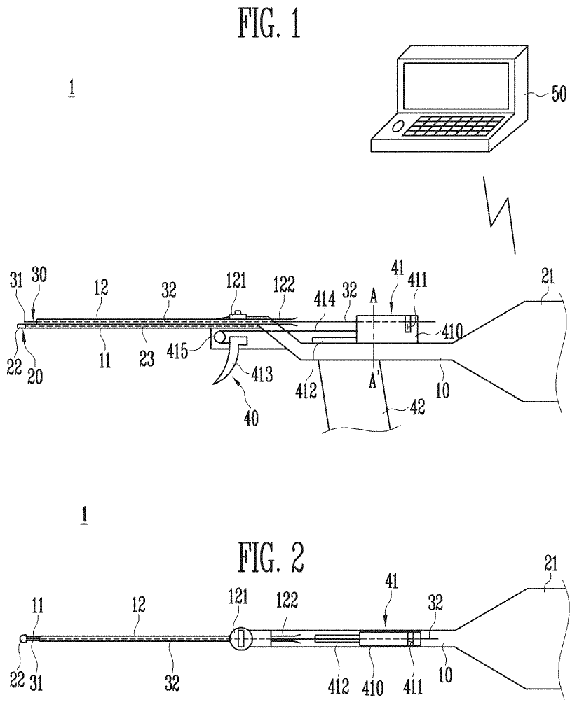

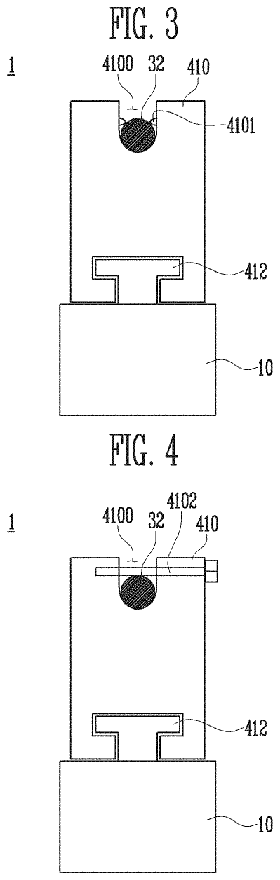

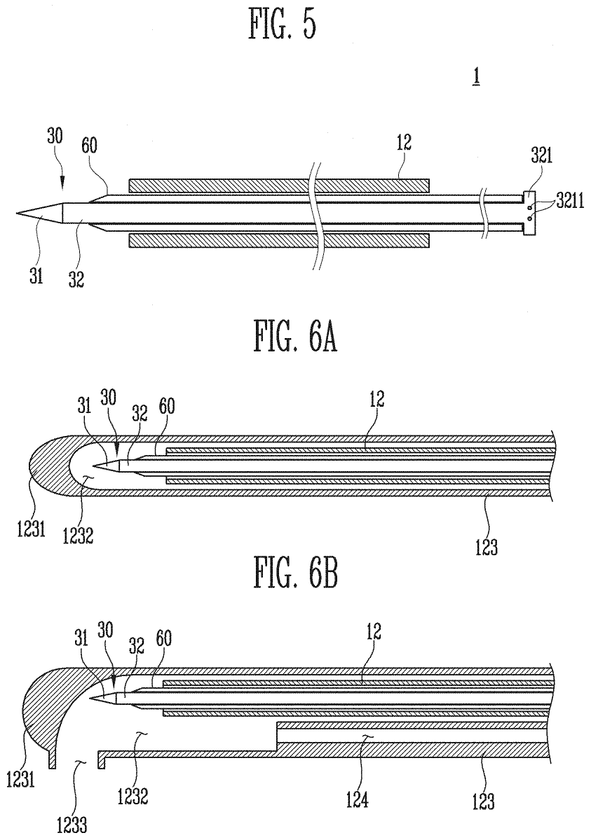

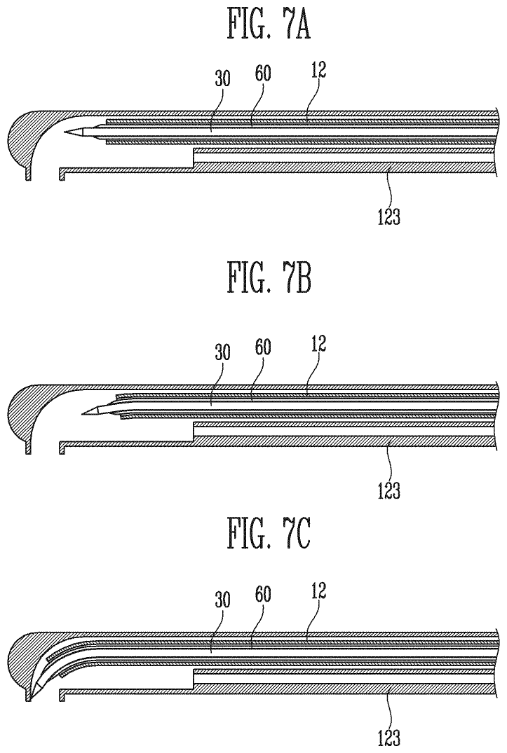

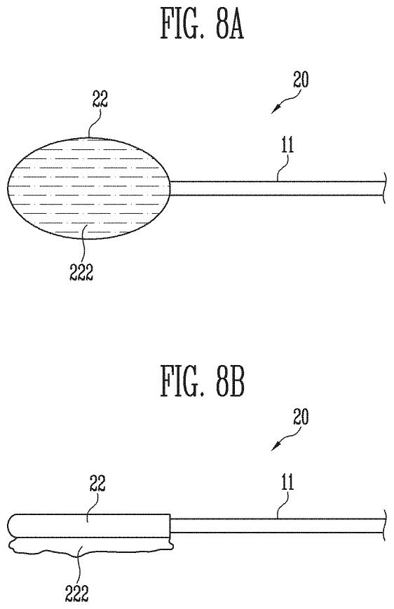

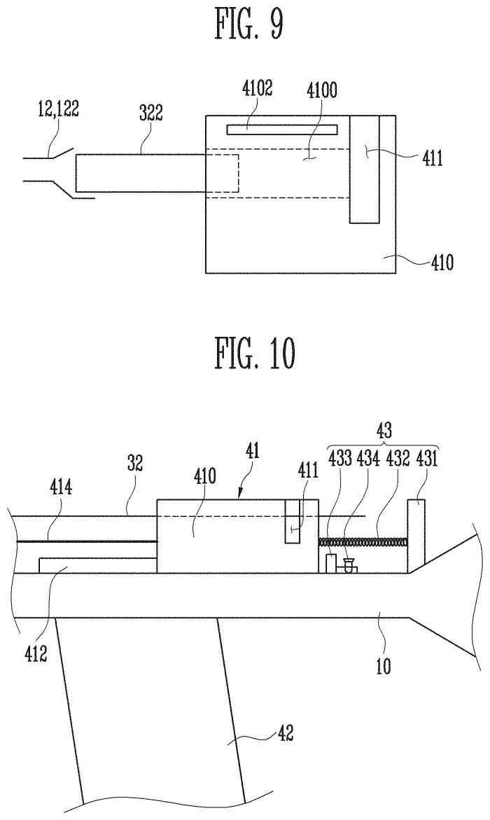

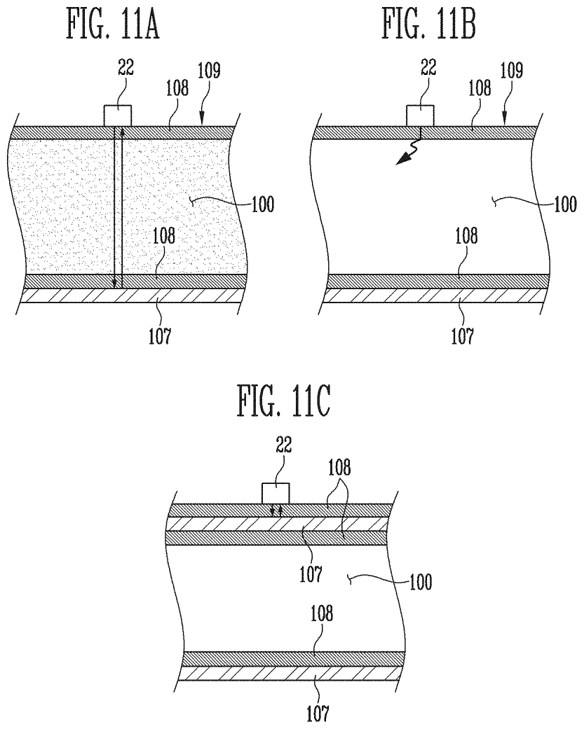

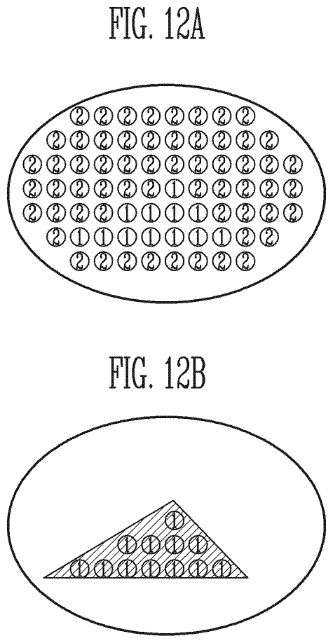

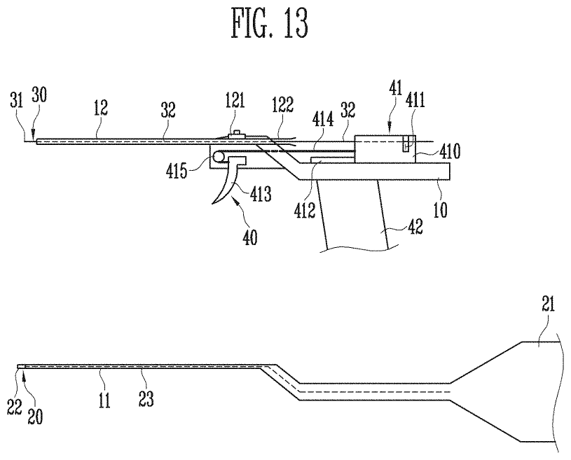

FIG. 1 is a side view of a maxillary sinus puncturing apparatus according to a first embodiment of the present invention; FIG. 2 is a plan view of the maxillary sinus puncturing apparatus according to the first embodiment of the present invention; and FIGS. 3 and 4 are cross-sectional views taken along line A-A′ of FIG. 1. In addition, FIG. 5 is a plan view of a puncturing tube in the maxillary sinus puncturing apparatus according to the first embodiment of the present invention; FIGS. 6A and 6B are a side cross-sectional view and a plan cross-sectional view, respectively, that illustrate another embodiment of a puncturing tube in the maxillary sinus puncturing apparatus according to the first embodiment of the present invention; FIGS. 7A to 7C are sequence diagrams for describing a method of operating the puncturing tube of FIG. 6; FIGS. 8A and 8B are a side view and a plan view, respectively, that illustrate another embodiment of an ultrasonic probe in the maxillary sinus puncturing apparatus according to the first embodiment of the present invention; FIG. 9 is a view illustrating a cautery wire protection tube in the maxillary sinus puncturing apparatus according to the first embodiment of the present invention; FIG. 10 is a view illustrating a moving block position adjuster in the maxillary sinus puncturing apparatus according to the first embodiment of the present invention; FIGS. 11A to 11C are views illustrating a method of detecting a posterior fontanelle by the maxillary sinus puncturing apparatus according to the first embodiment of the present invention; and FIGS. 12A and 12B are views illustrating a result detected in accordance with the method of detecting a posterior fontanelle of FIG. 11.

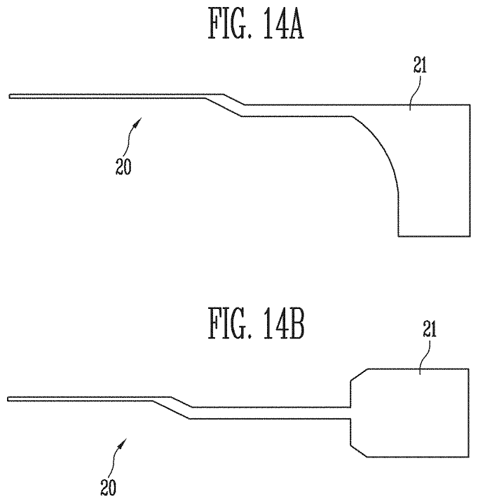

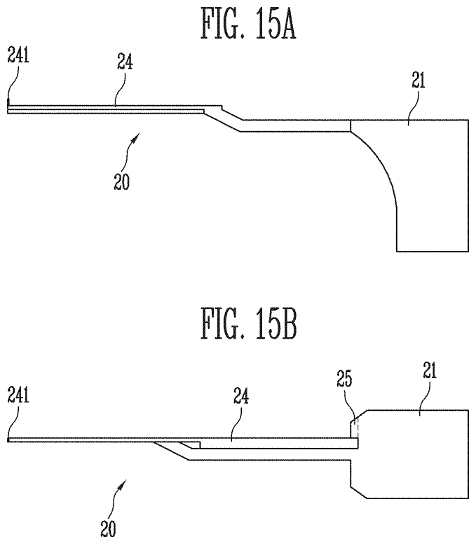

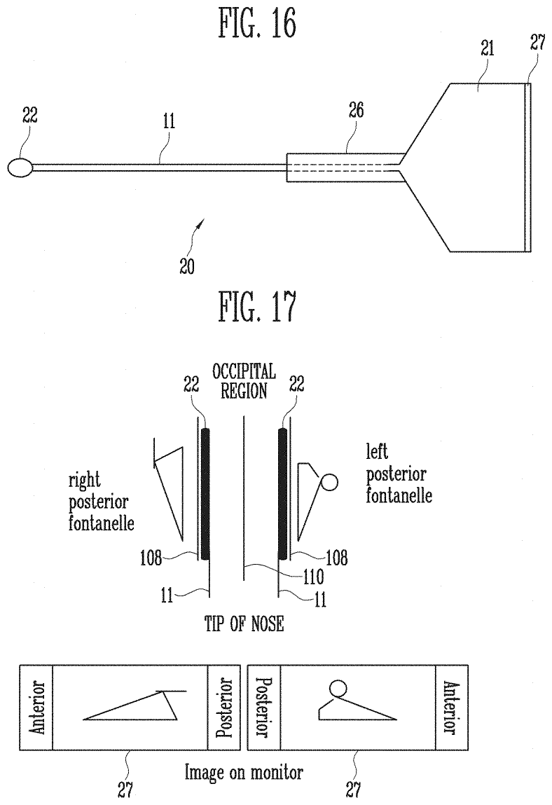

In addition, FIG. 13 is an exploded side view of the maxillary sinus puncturing apparatus according to the first embodiment of the present invention; FIGS. 14A and 14B are a side view and a plan view, respectively, that illustrate another embodiment of an ultrasonic probing unit in the maxillary sinus puncturing apparatus according to the first embodiment of the present invention; FIGS. 15A and 15B are a side view and a plan view, respectively, of the ultrasonic probing unit to which a probing unit cover is added; FIG. 16 is a view illustrating still another embodiment of an ultrasonic probing unit in the maxillary sinus puncturing apparatus according to the first embodiment of the present invention; and FIG. 17 is a view for describing an image shown on a probing monitor of the ultrasonic probing unit.

In addition, FIG. 23 is a side cross-sectional view of a face, and FIG. 24 is a front cross-sectional view of the face. FIGS. 23 and 24 may be utilized as references in the description of the present invention. A maxillary sinus 100, a superior meatus 101, a superior concha 102, a middle meatus 103, a middle concha 104, an inferior meatus 105, an inferior concha 106, a posterior fontanelle 109, etc. are shown in FIGS. 23 and 24.

Referring to FIGS. 1 to 17, a maxillary sinus puncturing apparatus 1 according to the first embodiment of the present invention includes a main body 10, an ultrasonic probing unit 20, a puncturing unit 30, an operation unit 40, a notification unit 50, and an irrigation tube 60.

The main body 10 is a portion gripped by an operator. The operator may detect the posterior fontanelle 109 using the ultrasonic probing unit 20, which will be described below, while gripping the main body 10 and then use the puncturing unit 30, which will be described below, to make a puncture toward the maxillary sinus 100.

The main body 10 may be in the shape of a rifle bent at least twice. This is to allow an index finger and a middle finger of the operator to perform different tasks while the operator is gripping the main body 10 and, at the same time, to facilitate securing a field of view during a surgical procedure. Here, the operator's middle finger may be used to pull a trigger 413 of the operation unit 40, which will be described below, and the index finger may be used to operate a rotating unit 121, which will be described below.

In the main body 10, a direction of insertion into the middle meatus 103 through a nostril may be defined as the front, and the opposite direction may be defined as the rear. Here, a probing tube 11 and a puncturing tube 12 may be provided at the front portion of the main body 10.

The probing tube 11 may be disposed in a lengthy form to be inserted into the middle meatus 103. The probing tube 11 is a tube provided for ultrasonic probing, and when taking into consideration that a typical distance from a nostril to the posterior fontanelle 109 is approximately 55 mm to 65 mm, the length of the probing tube 11 may be in a range of 80 mm to 100 mm. Also, the diameter of the probing tube 11 may be 1 to 3 mm.

An ultrasonic probe 22 configured to detect the posterior fontanelle 109 may be provided at the front end of the probing tube 11, and a coaxial lead wire 23 configured to transmit an electrical signal may be disposed inside the probing tube 11. Here, the coaxial lead wire 23 may have a front end connected to the ultrasonic probe 22 and a rear end connected to an ultrasonic main body 21, which will be described below. The ultrasonic main body 21 may be provided at a rear portion of the main body 10, and the probing tube 11 may have a structure in which the inner portion is connected to the rear portion of the main body 10.

The puncturing tube 12 may be provided in a lengthy form to be inserted into the middle meatus 103 together with the probing tube 11 and may be provided to be vertically parallel to the probing tube 11. The puncturing tube 12 is disposed to be vertically adjacent to the probing tube 11 since nasal cavities are sufficient in the vertical direction but are limited in the horizontal direction.

The length of the puncturing tube 12 may be substantially similar to the length of the probing tube 11, and the diameter thereof may be in a range of 1.0 mm to 1.5 mm. Also, the puncturing tube 12 may be provided so that a front end thereof is rotated by the rotating unit 121. The rotating unit 121 is provided in the main body 10 and may allow the front end of the puncturing tube 12 to be bent.

As illustrated in FIG. 5, a puncturing needle 31 may be provided at a front end of the puncturing tube 12, and a cautery wire 32 configured to transmit electricity to the puncturing needle 31 may be disposed inside the puncturing tube 12. Here, the cautery wire 32 may allow the puncturing needle 31 to protrude from the front end of the puncturing tube 12 due to back and forth movement of the cautery wire 32 inside the puncturing tube 12.

When the puncturing tube 12 is inserted toward the middle meatus 103, the posterior fontanelle 109 may be disposed at the left or the right of the puncturing tube 12. Therefore, a direction of the puncturing needle 31 should be changed in order to puncture the posterior fontanelle 109 using the puncturing needle 31. In the present invention, the direction of the puncturing needle 31 may be changed by causing the front end of the puncturing tube 12 to be bent using the rotating unit 121. A principle of a fiberscope which is widely being used in a conventional endoscope may be used here. However, detailed description of the structures of the rotating unit 121 and the puncturing tube 12 using a fiberscope will be omitted.

For example, the front end of the puncturing tube 12 may be bent rightward when the rotating unit 121 is rotated clockwise, the front end of the puncturing tube 12 may be bent leftward when the rotating unit 121 is rotated counterclockwise, and a locking unit (not marked) configured to fix the front end of the puncturing tube 12 in a bent state may be provided at the rotating unit 121.

The puncturing needle 31 provided at the front end of the puncturing tube 12 electrocauterizes the posterior fontanelle 109 using electricity to puncture the posterior fontanelle 109. Here, since heat is generated, the front end of the puncturing tube 12 may be insulated to be protected from the heat, and a method for insulating the front end of the puncturing tube 12 is not particularly limited.

In the above, the puncturing needle 31 punctures the posterior fontanelle 109 using electricity supplied by a puncturing electrocauterizer (not illustrated), and the puncturing electrocauterizer is turned off when the puncturing needle 31 punctures the posterior fontanelle 109 and enters the maxillary sinus 100. This is because pain or other side effects may be caused when the puncturing needle 31 comes into contact with mucosa 108 in the maxillary sinus 100 other than the posterior fontanelle 109, which is anesthetized, while the puncturing electrocauterizer is turned on.

An inlet portion 122 may be provided at the rear end of the puncturing tube 12. The probing tube 11 allows the ultrasonic probe 22 at the front end thereof to be connected up to the ultrasonic main body 21 at the rear end of the main body 10 via the coaxial lead wire 23, and the coaxial lead wire 23 does not need to be exposed to the outside of the probing tube 11 and the main body 10 at the rear end of the probing tube 11. However, the cautery wire 32 inserted into the puncturing tube 12 may come out to the outside of the puncturing tube 12 in order to be connected to the operation unit 40 or to be replaced.

Therefore, the cautery wire 32 may be inserted from the rear end of the puncturing tube 12 into the front end thereof through the inlet portion 122 of the puncturing tube 12. Here, the inlet portion 122 may be provided at the rear end of the puncturing tube 12, formed in the shape of a funnel having a cross-sectional area that gradually increases rearward, and form an inlet of the puncturing tube 12 for introducing the cautery wire 32. The inlet portion 122 is formed as above in order to allow the cautery wire 32 to be easily introduced into the puncturing tube 12.

The operator may detect the posterior fontanelle 109 using the ultrasonic probe 22 provided at the front end of the probing tube 11 and then puncture the posterior fontanelle 109 using the puncturing needle 31 provided at the front end of the puncturing tube 12. However, a spot coming into contact with the ultrasonic probe 22 and a spot coming into contact with the puncturing needle 31 may be slightly different.

The puncturing tube 12 generally has a property of being slightly flexible instead of being rigid when examined by itself. As illustrated in FIGS. 6A and 6B, in order to prevent the puncturing tube 12 from bending when being inserted into the narrow middle meatus 103, the puncturing tube 12 may include a sheath 123 configured to hold portions of the puncturing tube 12 except for the front end thereof that needs to be actually bent.

The sheath 123 may be formed in the shape of a rigid tube covering the puncturing tube 12. The sheath 123 will be described in detail below.

The sheath 123 may be configured such that a side at which the front end of the puncturing tube 12 is bent, i.e., a portion heading toward the posterior fontanelle 109, is relatively thick, and the remaining portions are relatively thin. By varying the thickness of the sheath 123 as above, a space for puncturing the posterior fontanelle 109 may be secured within the narrow middle meatus 103.

Since a surgical procedure needs to be performed on left and right sides of a patient in order to puncture the posterior fontanelle 109 with the puncturing unit 30, the sheath 123 is used while being rotated 180°. Here, in order to fix the sheath 123 to a position to be punctured after rotating the sheath 123, a method of forming a groove (not illustrated) on a case (not marked) surrounding a wire roller 415 and fitting a portion of the sheath 123 into the groove may be used. When rotating the sheath 123, the sheath 123 may be withdrawn from the groove, rotated in a desired direction, fitted into the groove again, and then fixed.

In addition, while holding the remaining portions of the puncturing tube 12 except for the front end thereof that needs to be actually bent, the sheath 123 includes a guide structure 1231, a sheath space 1232, and a sheath opening 1233 so that the front end of the puncturing tube 12 is not exposed to the outside, the front end of the puncturing tube 12 may be freely bent, and the front end of the puncturing tube 12 may accurately come into contact with a portion to be punctured of the posterior fontanelle 109.

The guide structure 1231 may form a front end of the sheath 123 and may be configured to prevent the front end of the puncturing tube 12 from being exposed to the outside.

The guide structure 1231 may have a convex outer shape to allow the puncturing tube 12 to be easily inserted into the narrow middle meatus 103 and a round inner shape to allow the puncturing needle 31 to be guided along the round surface to be accurately positioned at the portion to be punctured of the posterior fontanelle 109.

The sheath space 1232 provides a place at which the front end of the puncturing tube 12 may be bent, and a space region may be secured in the sheath space 1232 by the guide structure 1231.

The sheath opening 1233 may be formed by removing a portion of the sheath 123 at the side at which the front end of the puncturing tube 12 is bent, so that, when the posterior fontanelle 109 is punctured by the puncturing unit 30 or the irrigation tube 60 is inserted into the maxillary sinus 100, an end of the puncturing unit 30 partially exposed to the outside from the front end of the puncturing tube 12, i.e., an end of the puncturing needle 31 or the irrigation tube 60, passes through the sheath opening 1233.

An edge (not marked) protruding a predetermined length outward from an inlet portion may be further provided at the sheath opening 1233.

The edge forming the inlet of the sheath opening 1233 may not only serve to prevent the puncturing needle 31 or the irrigation tube 60 from being damaged due to the mucosa 108 being pushed into the sheath space 1232 through the sheath opening 1233 when the puncturing tube 12 is rotated toward the posterior fontanelle 109 after the puncturing tube 12 is inserted into the middle meatus 103, but also serve as a guide tube that allows the end of the puncturing needle 31 or the irrigation tube 60 that forms the end of the puncturing unit 30 to easily pass when the posterior fontanelle 109 is being punctured or irrigated.

The sheath 123 may include an endoscope insertion passage 124 to allow an endoscope to be used when puncturing the posterior fontanelle 109 by the puncturing unit 30. Here, in order to secure a sufficiently clear field of view when performing the surgical procedure, the sheath 123 may be manufactured with a transparent material.

The endoscope insertion passage 124 may be provided in the shape of a tunnel at the relatively thick portion of the sheath 123 in the longitudinal direction.

The endoscope insertion passage 124 may improve utilization of the maxillary sinus puncturing apparatus 1.

To assist in understanding the present invention, a method of operating the puncturing tube 12 including the sheath 123 may be briefly described as follows.

Before inserting the puncturing tube 12 including the sheath 123 into nasal cavities, the front end of the puncturing tube 12 is rotated toward the thickest side of the sheath 123 and fixed, and then the puncturing tube 12 including the sheath 123 is inserted into the nasal cavities. To allow the puncturing tube 12 including the sheath 123 to be easily inserted into the narrow middle meatus 103, the puncturing tube 12 including the sheath 123 is inserted while a side of the sheath 123 with the narrowest width is horizontal, a side of the sheath 123 with the widest width is vertical, and the front end of the puncturing tube 12 faces downward, and, when the front end of the sheath 123 reaches a position of the posterior fontanelle 109, the front end of the sheath 123 is rotated 90° such that the sheath opening 1233 turns toward the posterior fontanelle 109. Since, in reality, the puncturing needle 31 turns toward the inner portion of the maxillary sinus 100 even when the front end of the sheath 123 is slightly rotated rather than being rotated 90°, there is no problem in puncturing the maxillary sinus 100. Also, even when the sheath 123 receives resistance in the middle meatus 103 when the sheath 123 is being inserted thereinto, the sheath 123 is not damaged because the sheath 123 is rigid.

In other words, a space of the middle meatus 103 may be widened when the middle concha 104 is pushed toward a nasal septum, i.e., toward the center, using the sheath 123, and when the mucosa 108 in the middle meatus 103 receives a pressure and becomes thin, a space needed for a surgical procedure may be secured as much as the mucosa 108 is thinned. Even though the middle meatus 103 seems to be a narrow space, when the front end of the sheath 123 is rotated 90° after the sheath 123 is inserted into the middle meatus 103 while the side of the sheath 123 with the wide width is vertical, the rotation causing the above effects becomes possible.

Referring to FIGS. 7A to 7C, FIG. 7A illustrates an initial state of the puncturing tube 12 in which the puncturing tube 12 is inserted into the sheath 123. Here, since the puncturing needle 31 of the puncturing unit 30 is extremely rigid and does not bend, the puncturing needle 31 should not be inside the puncturing tube 12 that needs to bend and should remain exposed to the outside.

FIG. 7B illustrates a state in which the front end of the puncturing tube 12 is rotated. Here, due to a limitation in that it is not possible to sufficiently rotate the front end of the puncturing tube 12 toward the sheath opening 1233 in the narrow sheath space 1232, the puncturing needle 31 faces forward with a predetermined slope that is not vertical. When the front end of the puncturing tube 12 is rotated and bent at a predetermined angle, the bent state is maintained by being locked by the locking unit provided at the rotating unit 121.

FIG. 7C illustrates a state in which the puncturing tube 12 is moved forward toward the guide structure 1231 such that the puncturing needle 31 comes into contact with an inner surface of the guide structure 1231 and is guided toward the sheath opening 1233.

FIGS. 7A to 7C described above show the process before the puncturing tube 12 including the sheath 123 is inserted into the nasal cavities. After the process, the sheath 123 is inserted into the nasal cavities to allow the sheath opening 1233 to be located at a portion to be punctured of the posterior fontanelle 109, the sheath 123 receives the resistance of the guide structure 1231 as the puncturing tube 12 is continuously moved forward such that the puncturing needle 31 and the cautery wire 32 are bent further. In this way, the puncturing needle 31 may pass through the sheath opening 1233 in a vertical state and puncture the posterior fontanelle 109.

The ultrasonic probing unit 20 is a device configured to detect a punctured position of the posterior fontanelle 109 using ultrasonic waves. The ultrasonic probing unit 20 is provided in the main body 10 and detects the posterior fontanelle 109 where there are no bones 107 in a direction from the middle meatus 103 to the maxillary sinus 100. As mentioned in the description of the related art, since the bones 107 are punctured in a conventional method of puncturing the maxillary sinus 100, the method has problems in that the level of difficulty of the surgical procedure varies depending on the thickness of the bones 107 and there is a concern for damaging other portions. However, in the present invention, since the posterior fontanelle 109 formed only with the mucosa 108 without the bones 107 is detected in the direction from the middle meatus 103 to the maxillary sinus 100 using ultrasonic waves, and then the posterior fontanelle 109 is punctured, the level of difficulty and danger of the surgical procedure may be significantly lowered.

Here, a value detected by the ultrasonic probing unit 20 may be shown with an image and provided to the operator or indicated with a sound, a vibration, or the like. The detected value may be provided to the operator in various ways by the notification unit 50 which will be described below.

The ultrasonic probing unit 20 may include the ultrasonic main body 21, the ultrasonic probe 22, and the coaxial lead wire 23.

The ultrasonic main body 21 is provided at the rear portion of the main body 10 and generates an electrical signal for generating ultrasonic waves. Since the ultrasonic main body 21 has to have the size suitable for generating and analyzing an electrical signal, the ultrasonic main body 21 may be provided at the rear portion of the main body 10 which does not have to be inserted into the middle meatus 103. Of course the ultrasonic main body 21 may be provided at a position spaced apart from the main body 10 and connected to the main body 10 via a wire instead of being directly provided at the rear portion of the main body 10. Also, the ultrasonic main body 21 may be connected to the notification unit 50, which will be described below, via a wire or wirelessly and transmit a detected value to the notification unit 50.

The ultrasonic probe 22 is provided at the front portion of the main body 10. As described above, the ultrasonic probe 22 may be disposed at the front end of the probing tube 11. Here, the ultrasonic probe 22 may detect the posterior fontanelle 109 in the middle meatus 103 by converting an electrical signal into ultrasonic waves and emitting the ultrasonic waves.

Here, as illustrated in FIGS. 1 and 2, the ultrasonic probe 22 may be formed of a smooth, curved surface and have a form in which bent corner are minimized. This is to prevent the mucosa 108 in the nasal cavities from being damaged and to smoothly come into contact with the mucosa in the nasal cavities. The ultrasonic probe 22 may detect the posterior fontanelle 109 with corner portions at both sides.

Here, due to the ultrasonic probe 22 also coming into contact with portions other than the portion to be detected after entering the narrow nasal cavities, a confusion may be caused in the measurement. Therefore, a left corner and a right corner of the ultrasonic probe 22 may be made to operate independently. For example, when detecting the posterior fontanelle 109 in order to puncture the maxillary sinus 100 at a right side with respect to a patient, probing through the right corner of the ultrasonic probe 22 may be deactivated since the left corner of the ultrasonic probe 22 may be disposed in the direction of the posterior fontanelle 109, and vice versa. For this, a switch (not illustrated) configured to allow only one of the right or left side of the ultrasonic probe 22 to be activated may be provided in the main body 10 or the ultrasonic main body 21, and there may be one or more switches.

Unlike the probe described with reference to FIGS. 1 and 2, the ultrasonic probe 22 according to the present invention may employ an oval disk-shaped probe as illustrated in FIGS. 8A and 8B.

The oval disk-shaped ultrasonic probe 22 may generate ultrasonic waves from one side surface, and a liquid structure 222 may be further provided on the one side surface from which the ultrasonic waves are generated.

Since the oval disk-shaped ultrasonic probe 22 may detect the posterior fontanelle 109 by bringing the one side surface having a wide area into contact with the mucosa 108, the detection time may be shortened.

The oval disk-shaped ultrasonic probe 22 may be a probe that applies a single element transducer method, in which an electrical signal is converted into mechanical vibration by a part referred to as “piezoelectric crystal” to generate ultrasonic waves, the mechanical vibration, i.e., returning ultrasonic waves, is sensed, and then the sensed mechanical vibration is converted back to an electrical signal, or a dual element transducer method in which a transmitting element and a receiving element are separately provided.

Generally, since propagation of ultrasonic waves is possible only when the probe is adhered to an object without an air layer therebetween, when the oval disk-shaped ultrasonic probe 22 is used at the portion of the posterior fontanelle 109 which is not flat, the propagation of ultrasonic waves becomes possible only when the shape of a surface of the probe changes according to the shape of the posterior fontanelle 109 so that the surface of the probe is adhered to the mucosa. In the present embodiment, the liquid structure 222 allows the surface of the probe to be adhered to the mucosa.

A method of using the oval disk-shaped ultrasonic probe 22 is as follows. The mucosa 108 at a portion expected to be the posterior fontanelle 109 in the middle meatus 103 is marked with a marking needle, a central portion of the ultrasonic probe 22 is located at the marked portion of the mucosa 108, and the marked portion of the mucosa 108 is examined with ultrasonic waves to check the posterior fontanelle 109 through the notification unit 50, which will be described below, or a probing monitor 27, which will be described below. When a degree to which the position of the posterior fontanelle 109 identified by the ultrasonic waves is different from the portion marked with the marking needle is checked and then the actual posterior fontanelle 109 is punctured, the irrigation tube 60 may be safely inserted into the maxillary sinus 100.

The coaxial lead wire 23 transmits an electrical signal from the ultrasonic main body 21 to the ultrasonic probe 22. The coaxial lead wire 23 may be provided to pass through the inner portion of the probing tube 11 described above. That is, one end of the coaxial lead wire 23 may be connected to the ultrasonic probe 22 at the front end of the probing tube 11, and the other end of the coaxial lead wire 23 may be connected to the ultrasonic main body 21 at the rear portion of the main body 10.

The puncturing unit 30 is provided in the main body 10 and punctures the posterior fontanelle 109. Since the posterior fontanelle 109 corresponds to a portion formed only of the mucosa 109 and not having the bones 107, efficiency of a surgical procedure may be maximized in the present invention by allowing the irrigation tube 60, which is configured to irrigate the maxillary sinus 100, to be installed by passing through only the mucosa 108.

The puncturing unit 30 may include the puncturing needle 31 and the cautery wire 32.

The puncturing needle 31 corresponds to a puncturing electrocauterizer that uses electricity to cauterize the posterior fontanelle 109. The puncturing needle 31 may be manufactured in a bipolar type, etc. The puncturing tube 12 may be bent by the rotating unit 121 as described above, and a direction of the puncturing needle 31 may be adjusted by the bent front end of the puncturing tube 12.

However, for the front end of the puncturing tube 12 to be easily bent by the rotating unit 121, the puncturing needle 31 formed with a rigid material may be caused to protrude from the front end of the puncturing tube 12, before being inserted into a nostril, or in the inside of the sheath 123.

The cautery wire 32 supplies electricity to the puncturing needle 31. The cautery wire 32 may have a structure in which a wire connected to the puncturing needle 31 is coated with an insulator. The cautery wire 32 may be in a thin form so as to be movable inside the puncturing tube 12.

The cautery wire 32 may be formed with a material that causes the cautery wire 32 to be bent together with the puncturing tube 12 as the puncturing tube 12 is bent. However, a front end of the cautery wire 32 deviating from the front end of the puncturing tube 12 may be formed with a material capable of firmly supporting the puncturing needle 31.

That is, the front end of the cautery wire 32 may be formed of a material which is bent due to rotation of the puncturing tube 12 and has a strength capable of supporting the puncturing needle 31 coming out of the puncturing tube 12. Of course this may be realized by limiting the material of the cautery wire 32 and/or limiting a cross-sectional structure of the cautery wire 32, and the material or the cross-sectional structure is not particularly limited.

A locking protrusion 321 may be provided at the cautery wire 32. The irrigation tube 60 may be fitted into the cautery wire 32 disposed inside the puncturing tube 12. The locking protrusion 321 of the cautery wire 32 may come into contact with a rear end of the irrigation tube 60 in order to prevent the irrigation tube 60 from moving backward when the cautery wire 32 moves forward. Here, the locking protrusion 321 may be provided to be disposed within an open groove 411 which will be described below.

A cauterizing main body (not illustrated) configured to transmit electricity may be connected to a rear end of the cautery wire 32, and, like the ultrasonic main body 21 described above, the cauterizing main body may be provided in the main body 10 or may be provided to be spaced apart from the main body 10 and be connected to the main body 10 via a wire.

The cautery wire 32 may be manufactured to reach only up to the locking protrusion 321 and be fixed. Here, as illustrated in FIG. 5, a socket 3211 connected to the cautery wire 32 may be provided at the locking protrusion 321. The socket 3211 may be connected to or disconnected from an external wire and may be manufactured in various shapes at a portion of the locking protrusion 321. However, when a spring 432, which will be described below, is provided at a rear portion of a moving block 410, which will be described below, the socket 3211 is preferably provided at an upper end of the locking protrusion 321 as illustrated in FIG. 5 to avoid interfering with the spring 432.

In addition, a portion of the cautery wire 32 extending from the inlet portion 122 of the puncturing tube 12 to the moving block 410, which will be described below, may be exposed to the outside without being protected by the puncturing tube 12, and the exposed cautery wire 32 may be bent when the moving block 410 moves forward. Accordingly, as illustrated in FIG. 9, a cautery wire protection tube 322 may be further provided between the inlet portion 122 of the puncturing tube 12 and the moving block 410, which will be described below, in order to protect the exposed cautery wire 32.

The cautery wire protection tube 322 may have one end which is inserted and fixed to a fixing groove 4100 of the moving block 410, which will be described below, and extends to the inlet portion 122 of the puncturing tube 12 and may prevent the cautery wire 32 from being bent when the moving block 410 moves forward.

The operation unit 40 is provided in the main body 10 and moves the puncturing unit 30 toward the posterior fontanelle 109. The operation unit 40 is a configuration for allowing the puncturing needle 31 of the puncturing unit 30 to come into contact with the posterior fontanelle 109 to puncture the posterior fontanelle 109, and specifically, the operation unit 40 may allow the cautery wire 32 to move forward inside the puncturing tube 12.

The operation unit 40 may include a moving unit 41, a main body handle 42, and a moving block position adjuster 43.

The moving unit 41 may move the cautery wire 32 along the puncturing tube 12, and particularly, the moving unit 41 may move the cautery wire 32 forward along the puncturing tube 12. When the cautery wire 32 moves forward, the puncturing needle 31 connected to the front end of the cautery wire 32 may come into contact with the posterior fontanelle 109.

The moving unit 41 may include the moving block 410, a rail 412, and the trigger 413.

The moving block 410 moves the cautery wire 32, which is fixed thereto, so as to allow the cautery wire 32 to move along the inner portion of the puncturing tube 12. The fixing groove 4100 recessed from an upper surface of the moving block 410 in the longitudinal direction of the puncturing tube 12 may be provided in the moving block 410, and, when the cautery wire 32 is fitted into the fixing groove 4100, movement of the moving block 410 may be transmitted as it is to the cautery wire 32.

Here, in order to allow the cautery wire 32 to be fixed in a state of being fitted into the fixing groove 4100 of the moving block 410, a fixing protrusion 4101 may be provided at the fixing groove 4100 as illustrated in FIG. 3. The fixing protrusion 4101 may prevent the cautery wire 32 from being coming out of the fixing groove 4100 when the cautery wire 32 is completely inserted into the fixing groove 4100, and although, in this case, the fixing protrusion 4101 may of course serve as an obstacle in the process of inserting the cautery wire 32 into the fixing groove 4100, the cautery wire 32 may be completely inserted into the fixing groove 4100 over the fixing protrusion 4101 when the cautery wire 32 is pressed by applying a predetermined force.

A fixing member 4102 may be fitted into the fixing groove 4100 as illustrated in FIG. 4. That is, when the cautery wire 32 or the cautery wire protection tube 322 is inserted into the fixing groove 4100, and then the fixing member 4102 is fitted into the fixing groove 4100 by the operator so as to cross an upper side of the cautery wire 32, the cautery wire 32 or the cautery wire protection tube 322 may be prevented from being detached upward. Here, the fixing member 4102 may have a pin type structure or a plate type structure, one or more fixing members 4102 may be provided in the longitudinal direction of the cautery wire 32, and a groove (not marked) for inserting the fixing member 4102 may be provided in the moving block 410.

When the fixing member 4102 has a pin type structure, the fixing member 4102 may have a screw form having a screw thread or a simple pin form without a screw thread, and the groove may be provided in a shape corresponding thereto. When the fixing member 4102 is rotated by a predetermined angle, the state of the fixing member 4102 may be changed to a state in which it is able to come out of or is unable to come out of the groove so that the fixing member 4102 is fixed or separated. This may be realized by a structure in which a protrusion (not illustrated) is provided at a predetermined position in an outward direction from the fixing member 4102 and the fixing member 4102 is able to be inserted/withdrawn only when the corresponding protrusion is aligned to a specific position.

The open groove 411 may be provided at the moving block 410. The open groove 411 may be in the form of a groove that is recessed more than the fixing groove 4100 and may be opened sideward to allow the cautery wire 32 to be exposed from a side surface. Therefore, the operator may use the open groove 411 to check from the side surface whether the cautery wire 32 is properly fixed to the moving block 410. The open groove 411 may be provided at a middle point of the fixing groove 4100 provided from the front end to the rear end of the moving block 410 in the longitudinal direction.

In addition, the locking protrusion 321 of the cautery wire 32 may be fitted into the open groove 411. Since the cautery wire 32 at which the locking protrusion 321 is provided moves forward by the moving block 410 at which the open groove 411 is provided, the open groove 411 may have a horizontal width that allows the locking protrusion 321 to be fitted thereto. Here, the irrigation tube 60 may have a length in which the rear end thereof extends to the open groove 411.

The rail 412 is provided in the main body 10 and guides movement of the moving block 410. The rail 412 may have a T-shaped cross-section, and a rail groove (not marked) into which the rail 412 is inserted may be provided at the moving block 410 such that the moving block 410 may move back and forth while being fitted into the rail 412 without being detached therefrom.

Here, a roller or a sliding surface that facilitates movement of the moving block 410 may be provided at the rail 412, and the moving block 410 may remain spaced apart from an upper surface of the main body 10 while moving along an upper surface of the rail 412, so that the movement of the moving block 410 is prevented from being restricted due to friction.

The rail 412 may have a form that horizontally extends as much as a distance by which the cautery wire 32 has to move for the puncturing needle 31 to come into contact with the posterior fontanelle 109. However, a step (not illustrated) configured to prevent deviation of the moving block 410 may be provided on at least one of a front end and a rear end of the rail 412. Of course the step may be provided on the upper surface of the rail 412 and/or the upper surface of the main body 10.

The trigger 413 moves the moving block 410 on the rail 412 in accordance with the operator's manipulation. The trigger 413 may be connected to the moving block 410 via a wire 414, and the wire 414 may be wound around the wire roller 415 provided at a front portion of the trigger 413. When the trigger 413 is moved backward by the operator's middle finger or index finger, the wire 414 may be pulled as the wire roller 415 is rotated, and thus the moving block 410 may move forward.

Of course a structure and a principle in which the cautery wire 32 moves forward due to the pulling action of the trigger 413 are not limited to the above in the present invention. In the case of another embodiment of the moving unit 41, the cautery wire 32 may be moved forward due to a roller 416 instead of the moving block 410 when the trigger 413 is pulled. This will be described below.

The main body handle 42 may be provided at a portion of the main body 10 in the form of a handle of a pistol so that the operator may easily grip the main body 10.

The moving block position adjuster 43 may be installed at a rear portion of the moving block 410 as illustrated in FIG. 10 to adjust a position of the moving block 410 and includes a fixed wall 431, the spring 432, a preventive wall 433, and a screw 434.

The spring 432 may be installed between the rear end of the moving block 410 and the fixed wall 431 which is installed to be spaced a predetermined distance apart from the moving block 410.

The length of the cautery wire 32 may be slightly increased or decreased as the maxillary sinus puncturing apparatus 1 is used. The moving block position adjuster 43 is provided to compensate for the increased or decreased length and prevents the moving block 410 from moving from a predetermined position.

To manage the predetermined position of the moving block 410 more quantitatively, the preventive wall 433, which is configured to prevent further rearward movement of the moving block 410 even when the spring 432 pulls the moving block 410 from the rear of the moving block 410, is provided. The preventive wall 433 is fixed by the screw 434. A few preventive walls with different thicknesses may be prepared for the preventive wall 433, and, when the moving block 410 needs to be placed more forward or backward due to a changed length of the cautery wire 32, the preventive wall 433 may be replaced with a preventive wall corresponding to the changed length.

The notification unit 50 informs the operator of a value detected by the ultrasonic probing unit 20. The notification unit 50 may include a display for showing the value detected by the ultrasonic probe 22 with an image.

In addition, the notification unit 50 may inform the operator of the detected value in a simpler way. This will be described below with reference to FIGS. 11A to 11C and FIGS. 12A and 12B.

Referring to FIGS. 11A to 11C, the ultrasonic probe 22 may emit ultrasonic waves and then receive the returning ultrasonic waves. Here, when a duration of time from a point in time at which the ultrasonic waves are transmitted to a point in time at which the ultrasonic waves are received is measured, a distance of a portion to which the ultrasonic waves may be transmitted may be derived.

However, the ultrasonic waves have a property of being unable to pass through the bones 107 despite being able to pass through the mucosa 108 and has a property of being unable to be propagated when only air is present in the maxillary sinus 100 despite being able to be propagated when the maxillary sinus 100 is filled with a liquid such as secretions.

FIG. 11A is a case in which ultrasonic waves are emitted from the posterior fontanelle 109 formed only of the mucosa 108 without the bones 107 in the middle meatus 103 by the ultrasonic probe 22 while the maxillary sinus 100 is filled with a liquid such as secretions. The ultrasonic waves pass through the maxillary sinus 100 and then return due to the bones 107 inside the maxillary sinus 100, and here, the time taken for the ultrasonic waves to return may be expressed as a long time “Ta.”

FIG. 11B is a case in which ultrasonic waves are emitted from the posterior fontanelle 109 formed only of the mucosa 108 without the bones 107 in the middle meatus 103 by the ultrasonic probe 22 while only air is present in the maxillary sinus 100. The ultrasonic waves disappear within the maxillary sinus 100, and here, the time taken for the ultrasonic waves to return may be expressed as an infinite time “Tb.”

FIG. 11C is a case in which ultrasonic waves are emitted from portions where the bones 107 are present in the middle meatus 103 by the ultrasonic probe 22. The ultrasonic waves immediately return without passing through the maxillary sinus 100, and here, the time taken for the ultrasonic waves to return may be expressed as a short time “Tc.”

That is, like Ta, Tb, and Tc, differences may occur in time taken for recovering ultrasonic waves depending on whether the bones 107 are present in the spot from which the ultrasonic waves are emitted and an inner state of the maxillary sinus 100, and the ultrasonic probing unit 20 may detect the posterior fontanelle 109 using this principle. In this case, the notification unit 50 does not have to show the detected value with an image, and may instead confirm the user about whether the bones 107 are present and whether the maxillary sinus 100 is filled with a liquid or air, by using a simple numerical value that shows a distance reached by the ultrasonic waves or using different sounds/vibrations with respect to FIGS. 11A, 11B, and 11C. Here, since the notification unit 50 does not require a display, the size of the apparatus of the present invention may be reduced, and the notification unit 50 may be disposed within the ultrasonic main body 21.

That is, the operator may conveniently perform a surgical procedure by identifying a position of the posterior fontanelle 109 from the notification unit 50 while detecting the posterior fontanelle 109 by the ultrasonic probing unit 20 and then puncturing the posterior fontanelle 109.

As illustrated in FIGS. 12A and 12B, a result detected in accordance with the method of detecting the posterior fontanelle described with reference to FIG. 11 may be shown with an image on a probing monitor. As illustrated in FIG. 12A, a number “1” is displayed on the probing monitor when the time taken for recovering ultrasonic waves is either the long time “Ta” or the infinite time “Tb,” and a number “2” is displayed on the probing monitor when the time taken for recovering the ultrasonic waves is the short time “Tc.” When the long times “Ta” and the infinite times “Tb” are grouped by the number “1” and the short times “Tc” are grouped by the number “2” as above and colors of the numbers are varied, a form of the posterior fontanelle 109 may be shown with a color image on the probing monitor. In addition, as illustrated in FIG. 12B, the number “2” may be erased and only the number “1” may be left to show the form of the posterior fontanelle 109 with a geometric image on the probing monitor.

The irrigation tube 60 may be provided to be fitted into the cautery wire 32 in the puncturing tube 12 and may be installed at the posterior fontanelle 109 punctured by the puncturing needle 31. The irrigation tube 60 is a configuration for connecting the inside and the outside of the maxillary sinus 100, and, after the posterior fontanelle 109 is punctured by the puncturing unit 30, the irrigation tube 60 may remain installed in the punctured hole. The irrigation tube 60 is installed for irrigating the maxillary sinus 100 and may be used for the purpose of administering drugs as necessary.

The irrigation tube 60 may be manufactured using a material that is bendable while being rigid to a certain degree and is harmless to the human body, and there is no problem in irrigating the maxillary sinus 100 even when the diameter of the irrigation tube 60 is smaller than 1 mm. A front end portion of the irrigation tube 60 may be tapered similarly to a tube catheter used when performing an intravenous injection such that resistance is minimized when the irrigation tube 60 passes through the hole together with the puncturing needle 31.

The irrigation tube 60 is installed in the punctured hole of the posterior fontanelle 109. The puncturing needle 31 may move forward for puncturing the posterior fontanelle 109 and, after the puncturing, move forward for installing the irrigation tube 60. In the former case, the puncturing needle 31 moves forward only until it comes into contact with the posterior fontanelle 109; however, in the latter case, the puncturing needle 31 may move forward until it is inserted into the maxillary sinus 100.

In the latter case, when the puncturing needle 31 and the irrigation tube 60 enter the maxillary sinus 100 through the hole of the posterior fontanelle 109, the operator may move only the puncturing needle 31 and the cautery wire 32 backward. Here, the irrigation tube 60 may come out forward from the cautery wire 32 and remain in the hole.

Since the hole of the posterior fontanelle 109 formed by the puncturing needle 31 may naturally be blocked as time passes after the irrigation tube 60 is removed, the hole is not a problem. Also, even when the hole is not blocked, it is not a problem since there are cases in which an accessory ostium is present in a normal person.

The structure of the maxillary sinus puncturing apparatus 1 according to the present invention may be referred to as an integral type structure. However, the maxillary sinus puncturing apparatus 1 according to the present invention may also be configured to have a detachable type structure in which the ultrasonic probing unit 20 is detachable from the main body 10 so that puncturing the posterior fontanelle 109 or irrigating or administering drugs into the maxillary sinus 100 through the punctured portion in the narrow nasal cavities is facilitated and the posterior fontanelle 109 is easily detected. The maxillary sinus puncturing apparatus 1 having the detachable type structure will be described with reference to FIG. 13.

As illustrated in FIG. 13, the maxillary sinus puncturing apparatus having the detachable type structure may be formed of the main body 10 in which the puncturing unit 30 and the operation unit 40 are provided and the ultrasonic probing unit 20 which is detachable from the main body 10, and may separately perform detection or a puncturing procedure. The maxillary sinus puncturing apparatus 1 having the detachable type structure may of course perform the detection or the puncturing procedure in combination with each other.

When the maxillary sinus puncturing apparatus 1 has the integral type structure, the maxillary sinus puncturing apparatus 1 has to be configured to be able to rotate the sheath 123 surrounding the puncturing tube 12 in order to perform ultrasonic probing or puncturing on the left and right sides of a patient, and when the ultrasonic probe 22 is in the oval disk shape in which ultrasonic waves are generated from one side surface as illustrated in FIG. 8, the maxillary sinus puncturing apparatus 1 has to be configured to include a pair of ultrasonic probing units 20 at the left and right. However, the maxillary sinus puncturing apparatus 1 having the detachable type structure does not have to be configured to be able to rotate the sheath 123 surrounding the puncturing tube 12 in order to perform ultrasonic probing or puncturing on the left and right sides of the patient and may be configured to include only one ultrasonic probing unit 20, instead of the pair of ultrasonic probing units 20 at the left and right, regardless of the type of the ultrasonic probe 22.

In addition, while the front end of the puncturing tube 12 is configured to be bent 900 sideward from the direction of the trigger 413 in the maxillary sinus puncturing apparatus 1 manufactured as the integral type model, the front end of the puncturing tube 12 may be configured to be bent toward the trigger 413 in the maxillary sinus puncturing apparatus 1 manufactured as the detachable type model.

In the maxillary sinus puncturing apparatus 1 manufactured as the integral type model, when the puncturing tube 12 is inserted into the middle meatus 103, the puncturing unit 30 is inserted thereinto while the side of the sheath 123 with a wide width is vertical, i.e., while the trigger 413 and the main body handle 42 are vertical, and, when puncturing the posterior fontanelle 109, the main body handle 42 is rotated such that the sheath opening 1233 of the sheath 123 and the front end of the puncturing tube 12 turn toward the posterior fontanelle 109. Here, when the main body handle 42 is rotated 90° clockwise, the front end of the puncturing tube 12 turns toward the posterior fontanelle 109 at the right side of the patient, and, when the main body handle 42 is rotated 90° counterclockwise, the front end of the puncturing tube 12 turns toward the posterior fontanelle 109 at the left side of the patient. In this way, the posterior fontanelle 109 at the left side or the right side may be punctured.

In the maxillary sinus puncturing apparatus 1 manufactured as the detachable type model, although the ultrasonic probing unit 20 does not have the main body handle 42 that may be seen in the maxillary sinus puncturing apparatus manufactured as the integral type model, the ultrasonic probing unit 20 may be formed in a shape similar to the main body 10 as a whole and may be gripped as if holding a pencil and rotated to detect the posterior fontanelle 109 at the left side or the right side.

In the ultrasonic probing unit 20 which is detachable that is manufactured as the detachable type model, a groove (not illustrated) into which the rear end of the main body 10 may be fitted and fixed may be provided in a front surface portion of the ultrasonic main body 21 in order to facilitate the detachment of the ultrasonic probing unit 20 from the main body 10. Also, in order to prevent the probing tube 11 from deviating from the puncturing tube 12 when the ultrasonic probing unit 20 is attached to the main body 10, the ultrasonic probing unit 20 may be configured such that the probing tube 11 is coupled to the puncturing tube 12 while a rear portion of the probing tube 11 is surrounded by a clip (not illustrated).

In addition, as illustrated in FIGS. 15A and 15B, the ultrasonic probing unit 20 which is detachable that is manufactured as the detachable type model may include a probing unit cover 24 so that the ultrasonic probing unit 20 is protected while gripping the ultrasonic probing unit 20 is facilitated. The probing unit cover 24 extends to an upper portion of the probing tube 11, and this portion is preferably manufactured in a plate shape to reduce the volume.

At one end of the probing unit cover 24, i.e., a portion at which the ultrasonic probe 22 is disposed, a marking needle 241 may be provided to mark a portion of the posterior fontanelle 109 to be punctured using the puncturing unit 30. When the posterior fontanelle 109 is checked by the ultrasonic probe 22 and then the ultrasonic probing unit 20 is rotated 90°, the marking needle 241 leaves a scratch on the mucosa 108 of the posterior fontanelle 109 to mark a portion to be punctured.

To facilitate detachment of the probing unit cover 24, a coupling groove 25 may be provided in the front surface portion of the ultrasonic main body 21 so that a rear end of the probing unit cover 24 may be fitted thereto and fixed. Also, the ultrasonic probing unit 20 may be configured such that, in order to prevent the attached probing unit cover 24 from being detached from the probing tube 11, the probing unit cover 24 is coupled to the probing tube 11 while a rear portion of the probing unit cover 24 is surrounded by a clip (not illustrated).

In the maxillary sinus puncturing apparatus 1 manufactured as the integral type model or the maxillary sinus puncturing apparatus 1 manufactured as the detachable type model, the ultrasonic main body 21 may be manufactured in various shapes. However, when the socket 3211 is provided at the locking protrusion 321 as illustrated in FIG. 5, in order to eliminate an inconvenience when connecting an external wire to the socket 3211, an upper end of the ultrasonic main body 21 may be manufactured to be flat so as to be coplanar with the upper end of the main body 10 as illustrated in FIGS. 14A and 14B.

The ultrasonic probing unit 20 manufactured as the detachable type model may be configured to further include a probing unit handle 26 disposed at a front portion of the ultrasonic main body 21 and the probing monitor 27 disposed at a rear portion of the ultrasonic main body 21 as illustrated in FIG. 16.

When the probing unit handle 26 is gripped as if holding a pencil, the ultrasonic probing unit 20 manufactured as the detachable type model may perform ultrasonography in a rigid state. Here, when a surface of the probing unit handle 26 is angularly formed, the ultrasonic probing unit 20 may be prevented from arbitrarily rotating when being gripped.

The ultrasonic probe 22 may be an oval disk-shaped probe in which ultrasonic waves are generated from one side surface instead of being generated from left and right sides, e.g., ultrasonic waves are generated from one side surface as illustrated in FIG. 8. In order to examine the left side or the right side of the patient, when holding the probing unit handle 26, without any other manipulation, the probing unit handle 26 just has to be held while rotating the ultrasonic probing unit 20 by 180°. The ultrasonography may be started and ended using a button (not illustrated) or using a voice recognition method since the operator performs nasoendoscopy with one hand and uses the ultrasonic probing unit 20 with the other hand.

When the probing unit handle 26 is held while rotating the ultrasonic probing unit 20 by 180° about an axis of the probing unit handle 26, the ultrasonic probe 22 also turns toward the opposite direction, and the probing monitor 27 disposed at the rear portion of the ultrasonic main body 21 also rotates 180° simultaneously. Then, even without any other manipulation, the posterior fontanelle 109 may be naturally viewed in the form necessary for a surgical procedure from the probing monitor 27 as illustrated in FIG. 17. Generally, for an anatomical depiction, left and right structures are depicted as seen from the nasal septum 110, i.e., the center of the nasal cavities, and, since images shown on the probing monitor 27 are as above, there is no inconvenience in performing the surgical procedure. One pair of probing monitors 27 are seen in FIG. 17. One of the probing monitors 27 shows a state in which the other probing monitor 27 is rotated 180°. Here, “anterior” refers to a direction toward the tip of a nose, and “posterior” refers to a direction toward an occipital region.

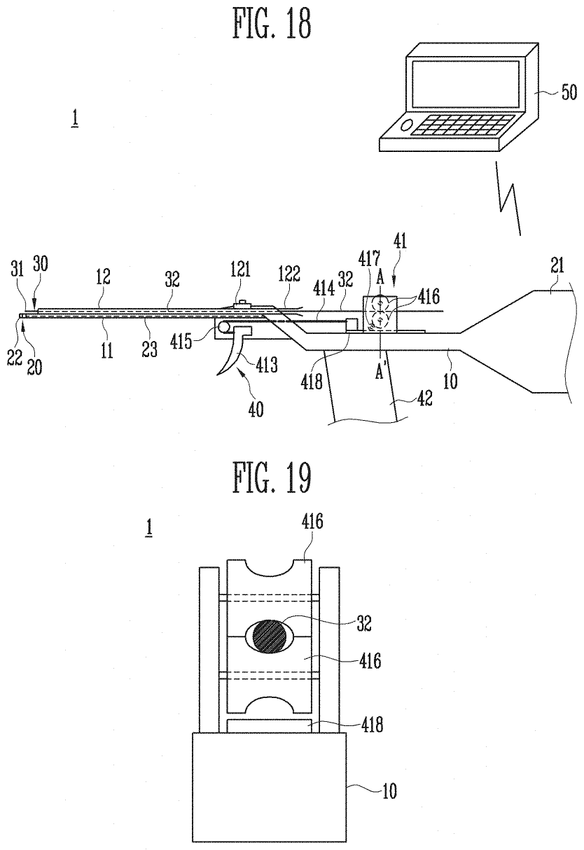

Hereinafter, another embodiment in which the moving unit 41 varies will be described with reference to FIGS. 18 and 19.

FIG. 18 is a side view illustrating another embodiment of a moving unit in the maxillary sinus puncturing apparatus according to the first embodiment of the present invention, and FIG. 19 is a cross-sectional view taken along line A-A′ of FIG. 18.

Referring to FIGS. 18 and 19, the moving unit 41 may include the roller 416, a rack 418, and the trigger 413. Hereinafter, differences from the first embodiment will be mainly described, and parts whose description is omitted should be referenced to the description of the first embodiment.

The roller 416 may be configured as one pair. The one pair of rollers 416 may rotate while the cautery wire 32 is disposed therebetween in order to allow the cautery wire 32 to move along the inner portion of the puncturing tube 12. As illustrated in the drawings, the rollers 416 may be vertically provided with respect to the cautery wire 32 or horizontally provided with respect to the cautery wire 32.

Portions of the rollers 416 coming into contact with the cautery wire 32 may be recessed to prevent the cautery wire 32 from deviating to the outside, and the two rollers 416 may come into contact with each other while the cautery wire 32 is disposed therebetween. In this case, when the two rollers 416 come into contact with each other, the size of the recessed portions may correspond to the cross-sectional area of the cautery wire 32. Also, the roller 416 may be formed with an elastic material and thus come into contact with both the cautery wire 32 and the other roller 416.

At least one of the pair of rollers 416 may be rotated by the trigger 413. Since the pair of rollers 416 may come into contact with each other, when any one roller 416 is rotated by the trigger 413, both of the rollers 416 may rotate and push the cautery wire 32 forward.

The rack 418 is directly or indirectly connected to the rollers 416 and realizes rotation of the rollers 416. The rack 418 may be disposed on the upper surface of the main body 10 and provided to move in the horizontal direction. The movement of the rack 418 may be realized by the principle and structure which are the same as or similar to those of the movement of the moving block 410 mentioned above.

The rack 418 may move forward when the trigger 413 is pulled, and, when the rack 418 moves forward, the rollers 416 may rotate, and the cautery wire 32 may move forward. Here, in order to align a direction in which the rack 418 moves and a direction in which the rollers 416 rotate, an auxiliary roller 417 may be connected between the rack 418 and the rollers 416. That is, the rack 418 may transmit a rotary force to the auxiliary roller 417, and the rollers 416 may come into contact with the auxiliary roller 417 and rotate. Therefore, when the rack 418 moves forward, the auxiliary roller 417 may move clockwise, the roller 416 at the lower side among the pair of rollers 416 may move counterclockwise, and the roller 416 at the upper side may come into contact with the roller 416 at the lower side and thus move clockwise. Therefore, the cautery wire 32 between the pair of rollers 416 moves forward.

The trigger 413 moves the rack 418 in accordance with the operator's manipulation to rotate the rollers 416. Since the principle of the wire 414 and the wire roller 415 described above in the first embodiment may be applied as it is to the moving of the rack 418 by the trigger 413, detailed description thereof will be omitted.

In this embodiment, the rollers 416, the auxiliary roller 417, etc. are configurations that rotate due to friction force. However, in the present invention, unlike the above, the rollers 416 and the auxiliary roller 417 may be substituted with gears or the like that rotate due to interlocking gear teeth. In this case, gear teeth may be provided at one surface of the rack 418, and the auxiliary roller 417 may be a rack gear interlocking with the rack 418.

Hereinafter, an order of a surgical procedure using the maxillary sinus puncturing apparatus 1 having the integral type structure will be briefly described.

While gripping the main body 10, the operator may check that the ultrasonic probe 22 and the coaxial lead wire 23 are provided in the probing tube 11 and that the puncturing needle 31 and the cautery wire 32 are provided in the puncturing tube 12.

Then, the operator may simultaneously insert the probing tube 11 and the puncturing tube 12 into the middle meatus 103, detect the posterior fontanelle 109 from the middle meatus 103 using the ultrasonic probe 22, and check a portion where there are no bones 107.

Then, in order to puncture the posterior fontanelle 109, the operator may adjust a direction of the front end of the puncturing tube 12 and pull the trigger 413. When the trigger 413 is pulled, the puncturing needle 31 and the cautery wire 32 move forward such that the puncturing needle 31 is located at a desired portion and a hole is formed by electrocauterization.

Then, in order to insert the irrigation tube 60 through the portion in which the hole is formed, the operator may pull the trigger 413 again to allow the puncturing needle 31 and the irrigation tube 60 to be inserted into the maxillary sinus 100 through the hole. Here, the trigger 413 may be pulled further than when the trigger 413 is pulled for puncturing the posterior fontanelle 109. The trigger 413 which is pulled for puncturing the posterior fontanelle 109 may return to an original position due to a spring (not illustrated) provided at the trigger 413, and then the trigger 413 may be pulled for inserting the irrigation tube 60.

Then, the operator may carefully take out only the puncturing needle 31 so that the irrigation tube 60, which is disposed in the hole, remains inside the maxillary sinus 100. By this process, the irrigation tube 60 communicating with the outside may be installed in the maxillary sinus 100.

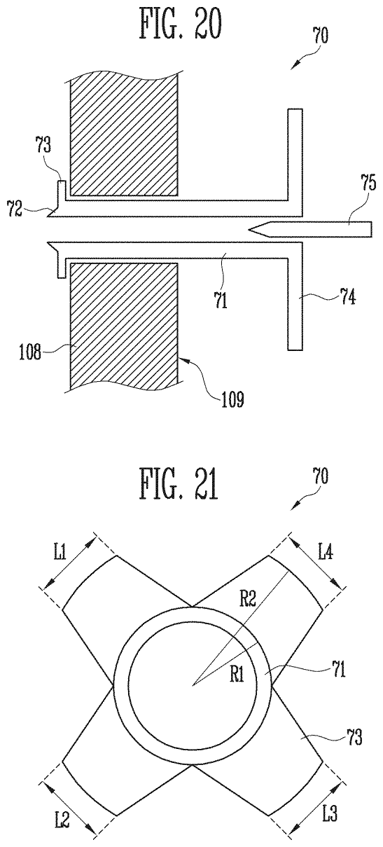

The maxillary sinus puncturing apparatus 1 according to the first embodiment may detect the posterior fontanelle 109 using the ultrasonic probing unit 20, puncture the posterior fontanelle 109 using the puncturing unit 30, and irrigate the maxillary sinus 100 or administer drugs into the maxillary sinus 100 using the irrigation tube 60. However, when it is determined that performing irrigation or drug administration one time using the irrigation tube 60 has only a marginal effect, the irrigation or the drug administration should be performed several times. Each time, removing the irrigation tube 60 and then continuously irrigating the maxillary sinus 100 or administering drugs into the maxillary sinus 100 through the hole formed in the posterior fontanelle 109 may be more preferable than continuing to puncture the posterior fontanelle 109. In the present invention, parts of the maxillary sinus puncturing apparatus 1 which are for addressing the above issue will be described with reference to FIGS. 20 to 22.

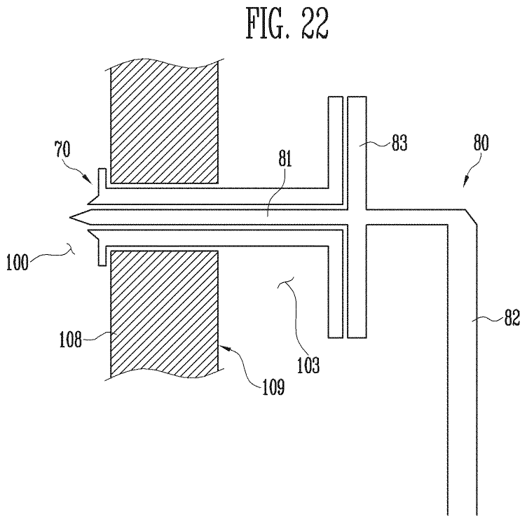

FIG. 20 is a view for describing a maxillary sinus tube which is a part of the maxillary sinus puncturing apparatus according to the present invention, FIG. 21 is a view for describing a tube front wing of the maxillary sinus tube, and FIG. 22 is a view for describing inserting and installing the maxillary sinus tube at a punctured portion of the posterior fontanelle using a tube insertion tool.

As illustrated in FIGS. 20 to 22, a maxillary sinus tube 70 which is a part of the maxillary sinus puncturing apparatus 1 includes a tube main body 71, a tube front end 72, a tube front wing 73, a tube hind wing 74, and a tube cover 75. The maxillary sinus puncturing apparatus 1 and the maxillary sinus tube 70 are medical devices.

The tube main body 71 may provide a passage for irrigating the maxillary sinus 100 or administering drugs into the maxillary sinus 100, may be manufactured in a size and shape corresponding to the hole formed in the posterior fontanelle 109, and may include the tube front wing 73, which will be described below, disposed at a front end and the tube hind wing 74, which will be described below, disposed at a rear end so that the tube main body 71 may remain inserted into a hole. Also, when the tube main body 71 having a large size is required, the size of the hole formed in the posterior fontanelle 109 may of course be widened.

The tube front end 72 is formed to become gradually thinner forward so that the tube main body 71 is easily inserted into the hole formed in the posterior fontanelle 109, i.e., the punctured portion of the posterior fontanelle 109.

The tube front wing 73 is provided at a front end of the tube main body 71 to allow the maxillary sinus tube 70 to remain inserted in the hole.

To facilitate insertion and removal of the tube main body 71, the tube front wing 73 may be configured of a plurality of wings by using a material that is bendable while being rigid to a certain degree.

Specifically, as illustrated in FIG. 21, for each of the plurality of tube front wings 73 to be easily folded when the maxillary sinus tube 70 is inserted and removed, it may be preferable that, while the plurality of wing portions are prevented from overlapping each other, the tube front wings 73 are manufactured such that a sum of lengths corresponding to an outer circle (radius R2) forming outer portions of the plurality of wings (L1+L2+L3+L4) is less than a circumference 2πR1 of an inner circle (radius R1) forming inner portions of the plurality of wings. Also, although the length of R2 is ideal when (R2−R1) is less than R1, since the maxillary sinus tube 70 has to be placed at a forward position in the posterior fontanelle 109, it may be preferable to not make the length of R2 too small in consideration of the size of the hole formed in the posterior fontanelle 109. Meanwhile, even when (R2−R1) is larger than R1, the length of R2 does not become a problem when the maxillary sinus tube 70 is inserted or removed, since the wing portions gather within the inner circle when the maxillary sinus tube 70 is being removed.

The tube hind wing 74 is provided at the rear end of the tube main body 71 to allow the maxillary sinus tube 70 to remain inserted in the hole together with the tube front wings 73.

The tube hind wing 74 has a size capable of preventing the maxillary sinus tube 70 from entering the maxillary sinus 100.

The tube cover 75 may block the maxillary sinus tube 70 inserted and installed through the hole formed in the posterior fontanelle 109 so as to block between the maxillary sinus 100 and the middle meatus 103 and may be removed when irrigating the maxillary sinus 100 or administering drugs into the maxillary sinus 100.

As illustrated in FIG. 22, a tube insertion tool 80 may be used in order to insert and install the maxillary sinus tube 70 in the hole formed in the posterior fontanelle 109, i.e., in the punctured portion of the posterior fontanelle 109.

The tube insertion tool 80 includes a tool main body 81, a tool handle 82, and a support part 83.

A method of inserting the maxillary sinus tube 70 using the tube insertion tool 80 is as follows. When a force is applied to the tool handle 82 while the tool main body 81 is fitted into the tube main body 71 of the maxillary sinus tube 70, as the force is transmitted to the tube hind wing 74 by the support part 83, the maxillary sinus tube 70 is inserted into the punctured portion. Then, the tube insertion tool 80 is removed from the maxillary sinus tube 70 and stored.

The maxillary sinus tube 70 inserted and installed in the punctured portion of the posterior fontanelle 109 as above has to be removed when irrigating the maxillary sinus 100 or administering drugs into the maxillary sinus 100 is not necessary. The maxillary sinus tube 70 may be removed by holding the tube main body 71 using forceps and applying a force toward the nasal septum (the center).

As described above, the maxillary sinus tube 70 may be inserted and installed through the hole formed in the posterior fontanelle 109 after the irrigation tube 60 is removed, thereby allowing the irrigation or drug administration to be performed several times as necessary. Also, since the maxillary sinus 100 is a wide hollow space and thus may provide a place in which drugs may be stored, in the future, the maxillary sinus 100 might be used as a drug storage place not only when treating maxillary sinusitis but also when drugs need to be continuously administered for a long period of time due to other diseases. For this, when drugs are injected into the maxillary sinus 100 and the maxillary sinus tube 70 is blocked by the tube cover 75 while the maxillary sinus tube 70 is installed in the hole formed in the posterior fontanelle 109, the drugs injected into and stored in the maxillary sinus 100 are continuously supplied to the nasal cavities through the maxillary sinus ostium and are supplied to a stomach via a nasopharynx, a pharynx, and an esophagus.

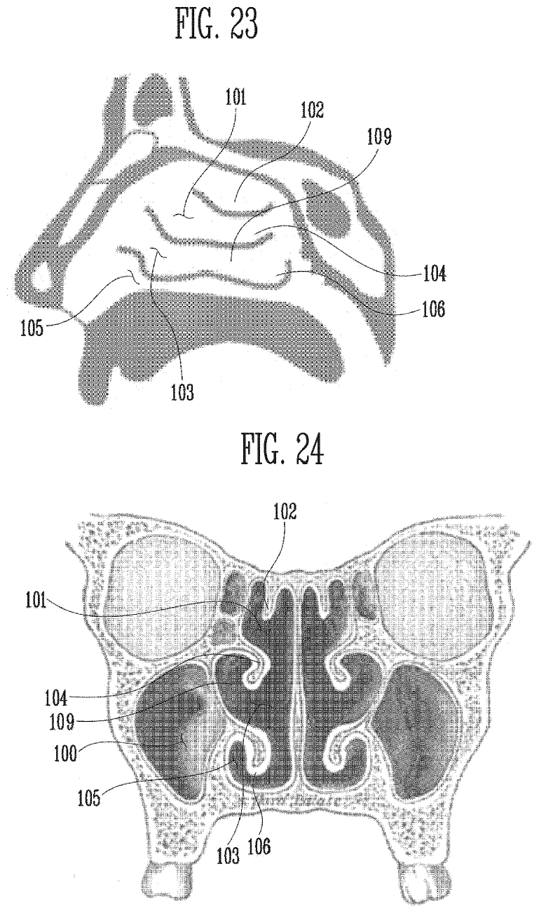

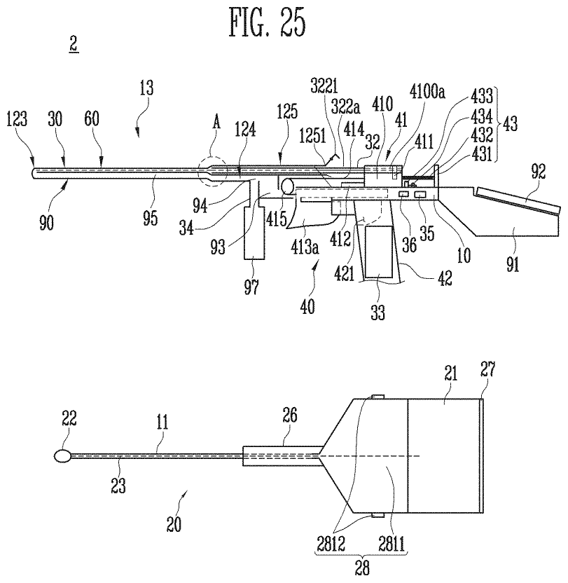

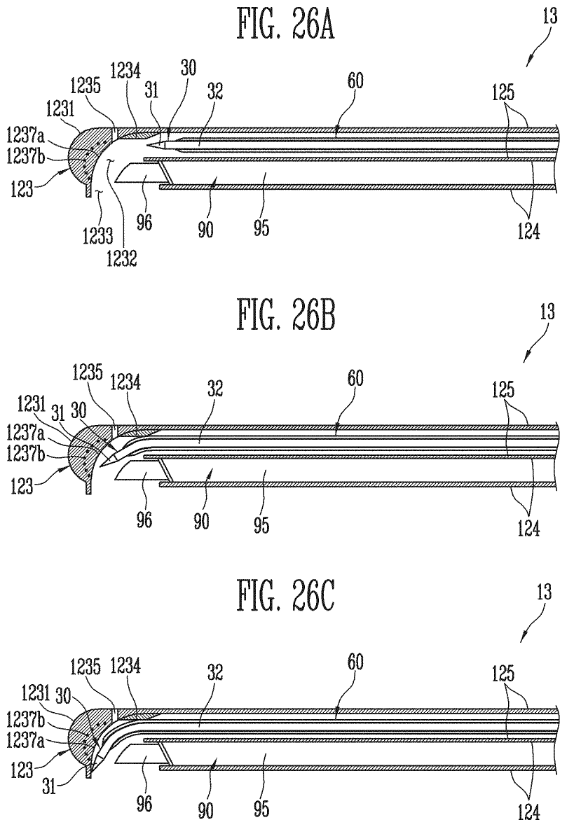

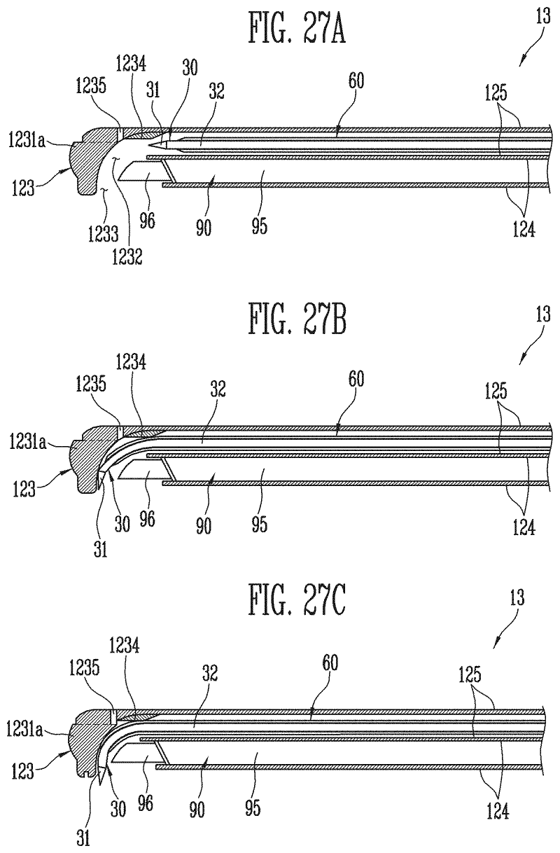

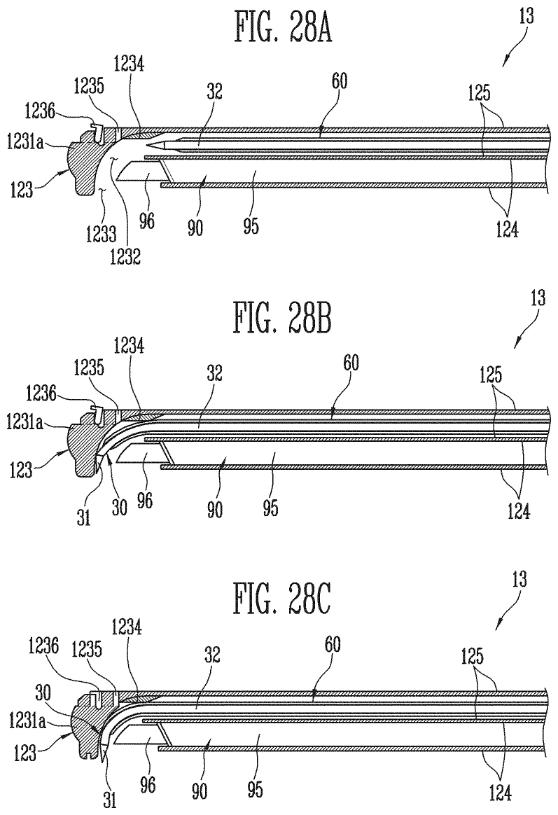

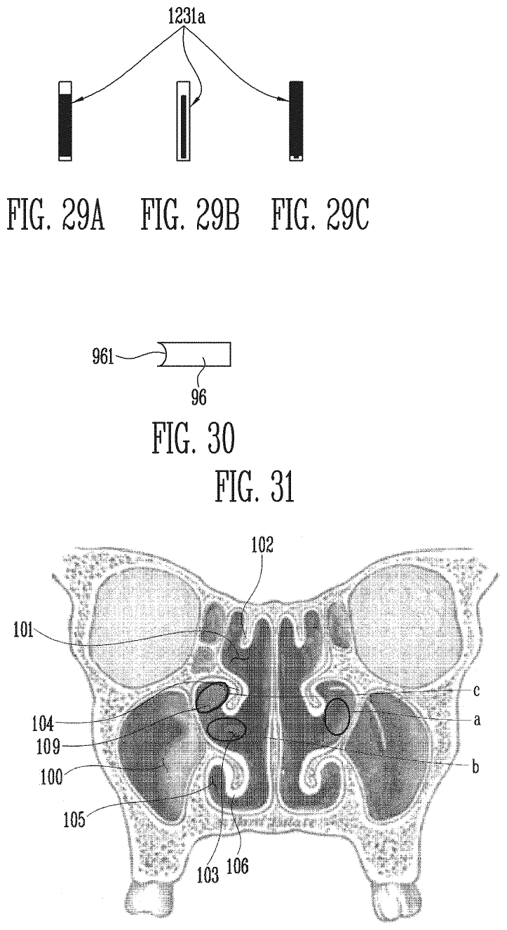

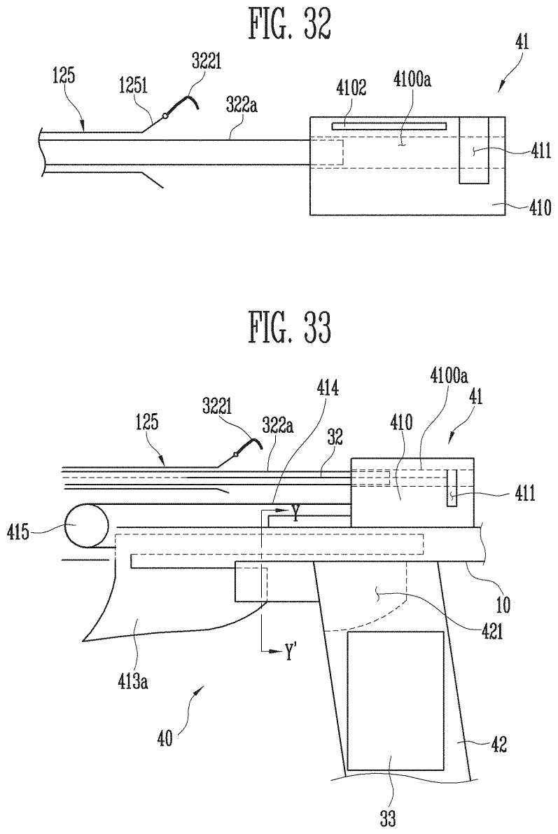

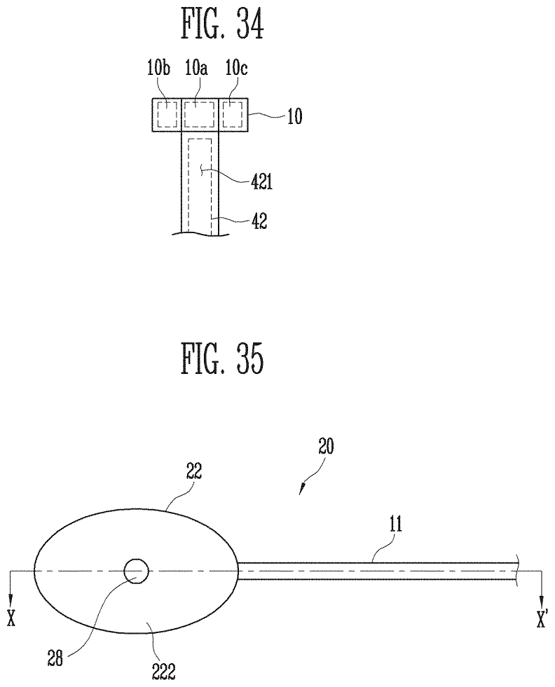

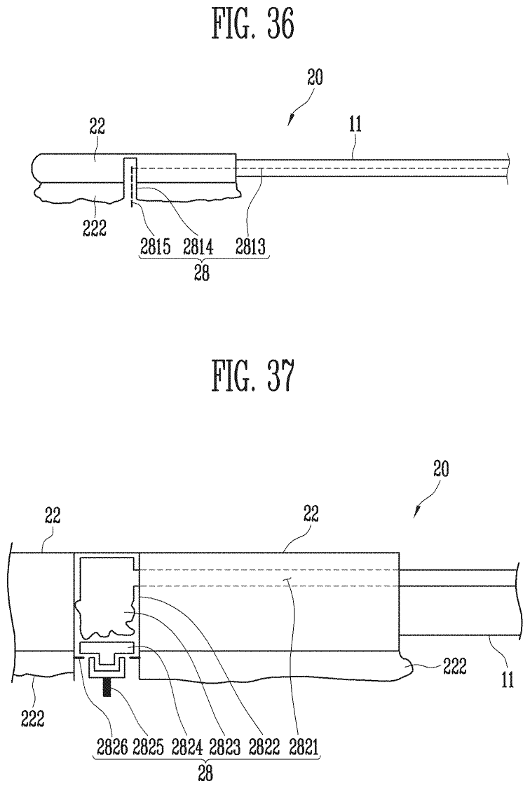

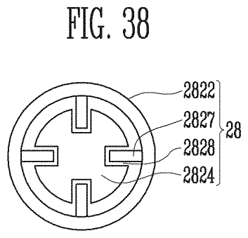

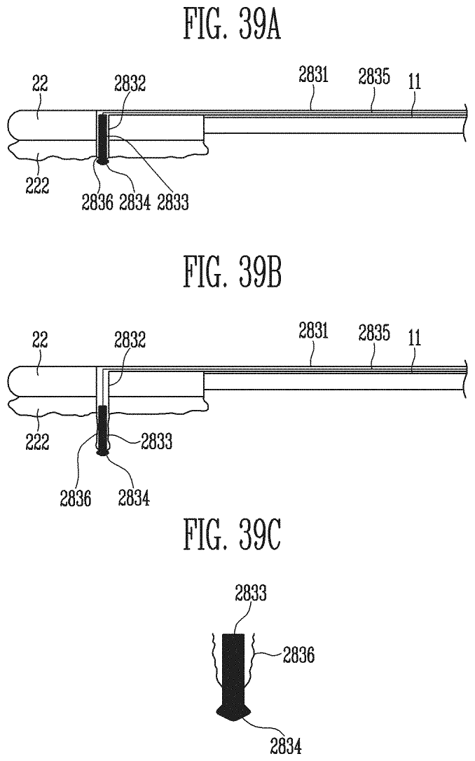

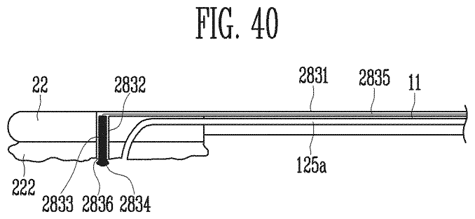

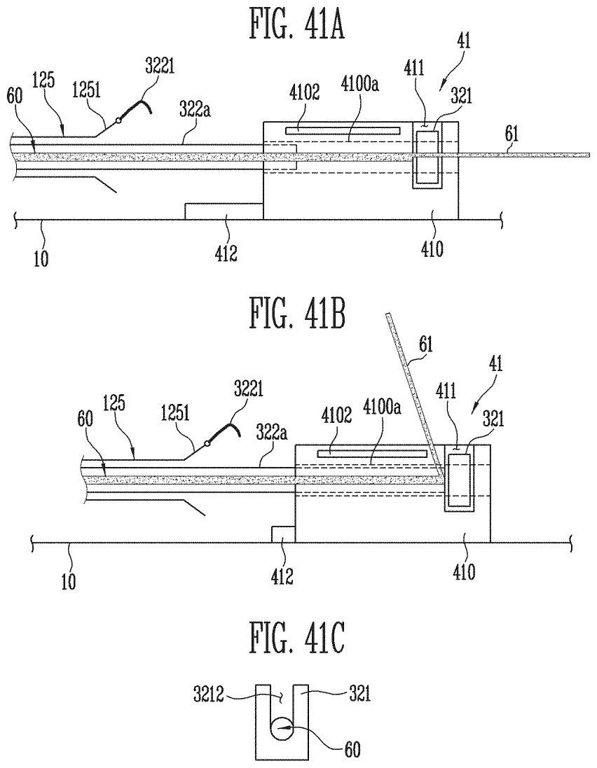

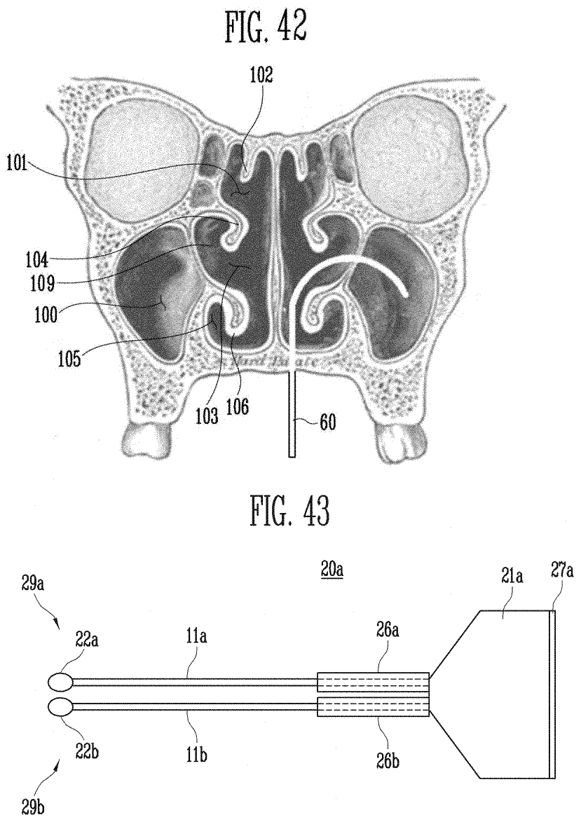

FIG. 25 is an exploded view for describing a maxillary sinus puncturing apparatus according to a second embodiment of the present invention; FIGS. 26A to 26C are views for describing an embodiment of a surgical procedure tube in the maxillary sinus puncturing apparatus according to the second embodiment of the present invention; FIGS. 27A to 27C are views for describing another embodiment of a surgical procedure tube in the maxillary sinus puncturing apparatus according to the second embodiment of the present invention; FIGS. 28A to 28C are views for describing still another embodiment of a surgical procedure tube in the maxillary sinus puncturing apparatus according to the second embodiment of the present invention; FIGS. 29A to 29C are views for describing the shape of a movable guide structure of FIGS. 27 and 28; FIG. 30 is a view for describing the shape of an endoscope window of FIGS. 27 and 28; FIG. 31 is a front cross-sectional view of a face for describing the fact that the surgical procedure tube is disposed in the posterior fontanelle; FIG. 32 is an enlarged view of portions of a cautery wire protection tube and a moving unit in the maxillary sinus puncturing apparatus according to the second embodiment of the present invention; FIG. 33 is an enlarged view of a portion of a trigger in the maxillary sinus puncturing apparatus according to the second embodiment of the present invention; FIG. 34 is a cross-sectional view taken along line Y-Y′ of FIG. 33; FIG. 35 is a view illustrating an ultrasonic probing unit coupled to a marking unit in the maxillary sinus puncturing apparatus according to the second embodiment of the present invention; FIG. 36 is a cross-sectional view taken along line X-X′ of FIG. 35 that is for describing a first embodiment of the marking unit in the maxillary sinus puncturing apparatus according to the second embodiment of the present invention; FIG. 37 is a cross-sectional view taken along line X-X′ of FIG. 35 that is for describing a second embodiment of the marking unit in the maxillary sinus puncturing apparatus according to the second embodiment of the present invention; FIG. 38 is a view for describing a coupling structure between a piston and a cylinder tube in FIG. 37; FIGS. 39A to 39C are cross-sectional views taken along line X-X′ of FIG. 35 that are for describing a third embodiment of the marking unit in the maxillary sinus puncturing apparatus according to the second embodiment of the present invention; and FIG. 40 is a cross-sectional view taken along line X-X′ of FIG. 35 that is for describing a fourth embodiment of the marking unit in the maxillary sinus puncturing apparatus according to the second embodiment of the present invention. FIGS. 41A to 41C are views for describing an irrigation tube, and FIG. 42 is a front cross-sectional view of a face illustrating a state in which the irrigation tube is inserted into the maxillary sinus.

Hereinafter, the second embodiment will be described with reference to FIGS. 25 to 42. Note that configurations using the reference numerals same as those in the first embodiment do not necessarily refer to the configurations same as those in the first embodiment.

In addition, the operating principle of a maxillary sinus puncturing apparatus 2 according to the second embodiment is basically similar to that of the maxillary sinus puncturing apparatus 1 according to the first embodiment, but an operating method of the maxillary sinus puncturing apparatus 2 may be somewhat different from that of the maxillary sinus puncturing apparatus 1 due to a structural difference. Detailed description of parts overlapping with the first embodiment will be omitted.

In addition, the maxillary sinus puncturing apparatus 2 according to the second embodiment is configured so that the puncturing tube 12 of the maxillary sinus puncturing apparatus 1 according to the first embodiment may be eliminated. In this way, the cost of manufacturing the puncturing tube 12 is saved, and a difficulty in manufacturing the puncturing tube 12 is alleviated.

Referring to FIGS. 25 to 42, the maxillary sinus puncturing apparatus 2 according to the second embodiment of the present invention includes a main body 10, a first surgical procedure tube 13, an operation unit 40, an ultrasonic probing unit 20, and a marking unit 28, and, here, the first surgical procedure tube 13 includes a sheath 123, a puncturing unit 30, an irrigation tube 60, and an endoscope unit 90.

The case in which the maxillary sinus puncturing apparatus 2 according to the second embodiment is manufactured as a detachable type model in which the ultrasonic probing unit 20 is detachable from the main body 10 will be described below. However, the maxillary sinus puncturing apparatus 2 is not limited to the detachable type model, and, by combinations with the above-described first embodiment, the maxillary sinus puncturing apparatus 2 may of course be realized in various other models, e.g., an integral type model, a removable type model, and the like.

The main body 10 is a portion gripped by the operator. While gripping the main body 10, the operator allows the maxillary sinus 100 to be punctured using the puncturing unit 30, which will be described below, and allows the maxillary sinus 100 to be irrigated using the irrigation tube 60, which will be described below.

The main body 10 may be manufactured in various shapes to allow an index finger and a middle finger of the operator to perform different tasks while the operator is gripping the main body 10 and, at the same time, to facilitate securing a field of view during a surgical procedure. As illustrated in FIG. 34, a first passage 10a configured to guide horizontal movement of a trigger 413a, a second passage 10b for installing a component related to the endoscope unit 90, and a third passage 10c for installing a component related to the puncturing unit 30 may be provided in the main body 10.

In the main body 10, a direction of insertion into the middle meatus 103 through a nostril may be defined as the front, and the opposite direction may be defined as the rear. Here, the first surgical procedure tube 13 may be provided at the front portion of the main body 10.

In order to allow a posterior fontanelle 109 to be punctured and the maxillary sinus 100 to be irrigated while the posterior fontanelle 109 is monitored, the first surgical procedure tube 13 may include the sheath 123, the puncturing unit 30, the irrigation tube 60, and the endoscope unit 90 and may be provided in a lengthy form to be inserted into the middle meatus 103.

The sheath 123 may have a tubular shape that forms an exterior of the first surgical procedure tube 13 while accommodating the puncturing unit 30, the irrigation tube 60, and the endoscope unit 90. Hereinafter, the sheath 123 will be described in detail with reference to FIGS. 26, 27, and 28.

The sheath 123 may be configured so that a portion heading toward the posterior fontanelle 109 is relatively thick and the remaining portions are relatively thin. By varying the thickness of the sheath 123 as above, a space for puncturing the posterior fontanelle 109 may be secured within the narrow middle meatus 103.

In order to allow a surgical procedure to be performed on left and right sides of a patient for puncturing the posterior fontanelle 109 with the puncturing unit 30, irrigating the posterior fontanelle 109 with the irrigation tube 60, or monitor the posterior fontanelle 109 with the endoscope unit 90, the sheath 123 may be configured to be used by being rotated 180°. Here, in order to fix the sheath 123 to a position at which the puncturing, irrigating, or monitoring is to be performed after rotating the sheath 123, a method of forming a groove (not illustrated) on a case (not marked) surrounding a wire roller 415 and fitting a portion of the sheath 123 into the groove may be used. When rotating the sheath 123, the sheath 123 may be withdrawn from the groove, rotated in a desired direction, fitted into the groove again, and then fixed.

FIGS. 26A to 26C are views for describing an embodiment of the first surgical procedure tube 13 in the maxillary sinus puncturing apparatus 2 according to the second embodiment of the present invention. FIG. 26A illustrates an initial state of the first surgical procedure tube 13 in which, together with the irrigation tube 60, the puncturing unit 30 is inserted into a surgical procedure means insertion passage 125 of the sheath 123; FIG. 26B illustrates a state in which a front end of the puncturing unit 30 is rotated; and FIG. 26C illustrates a state in which the puncturing unit 30 is moved forward toward a fixed guide structure 1231 such that the puncturing needle 31 comes into contact with an inner surface of the fixed guide structure 1231 and is guided toward the sheath opening 1233.

FIGS. 26A to 26C described above show the process before the sheath 123 is inserted into the nasal cavities. After the process, the sheath 123 is inserted into the nasal cavities to allow the sheath opening 1233 to be located at a portion to be punctured of the posterior fontanelle 109, the sheath 123 receives the resistance of the fixed guide structure 1231 as the puncturing unit 30 is continuously moved forward such that the puncturing needle 31 and the cautery wire 32 are bent further. In this way, the puncturing needle 31 may pass through the sheath opening 1233 in a vertical state and puncture the posterior fontanelle 109.

Referring to FIG. 26, the sheath 123 includes the fixed guide structure 1231, the sheath space 1232, the sheath opening 1233, a direction-changing protrusion 1234, an emergency treatment hole 1235, an endoscope insertion passage 124, and the surgical procedure means insertion passage 125 so that the front ends of the puncturing unit 30, the irrigation tube 60, and the endoscope unit 90 are not exposed to the outside, the front end of the puncturing unit 30 may be freely bent, and the front end of the puncturing unit 30 may accurately come into contact with a portion to be punctured of the posterior fontanelle 109.

The fixed guide structure 1231 which is a fixed type may form the front end of the sheath 123 and be configured so that the front ends of the puncturing unit 30, the irrigation tube 60, and the endoscope unit 90 are not exposed to the outside.

The fixed guide structure 1231 may have a convex outer shape to allow the puncturing unit 30 to be easily inserted into the narrow middle meatus 103 and a round inner shape to allow the puncturing needle 31 to be guided along a first round surface 1237a to be accurately positioned at the portion to be punctured of the posterior fontanelle 109.

The sheath space 1232 provides a place at which the front end of the puncturing unit 30 may be bent, and a space region may be secured in the sheath space 1232 by the fixed guide structure 1231.

The sheath opening 1233 may be formed by removing a portion of the sheath 123 at the side at which the front end of the puncturing unit 30 is bent, so that, when the posterior fontanelle 109 is punctured by the puncturing unit 30 or the irrigation tube 60 is inserted into the maxillary sinus 100, an end of the puncturing unit 30, i.e., an end of the puncturing needle 31 or the irrigation tube 60, passes through the sheath opening 1233.

An edge (not marked) protruding a predetermined length outward from an inlet portion may be further provided at the sheath opening 1233.

The edge forming the inlet of the sheath opening 1233 may not only serve to prevent the puncturing needle 31 or the irrigation tube 60 from being damaged due to the mucosa 108 being pushed into the sheath space 1232 through the sheath opening 1233 when the puncturing unit 30 is rotated toward the posterior fontanelle 109 after the puncturing unit 30 is inserted into the middle meatus 103, but also serve as a guide tube that allows the end of the puncturing needle 31 or the irrigation tube 60 that forms the end of the puncturing unit 30 to easily pass when the posterior fontanelle 109 is being punctured or irrigated.

The direction-changing protrusion 1234 guides the front end of the puncturing unit 30 to be easily bent toward the sheath opening 1233 when the puncturing unit 30 moves forward to puncture the posterior fontanelle 109. The direction-changing protrusion 1234 may be further provided at an upper portion of an inner peripheral surface of the surgical procedure means insertion passage 125, which is upper portion of an inner peripheral surface of the sheath 123. Here, the direction-changing protrusion 1234 serves as the puncturing tube 12, which causes the puncturing unit 30 to be bent, of the above-described first embodiment.

In order to guide the puncturing needle 31 of the puncturing unit 30 to be bent toward the sheath opening 1233 before the puncturing needle 31 comes into contact with the first round surface 1237a of the guide structure 1231, the direction-changing protrusion 1234 may be provided at a boundary portion between a round surface of the sheath 123 and a linear surface of the sheath 123. The puncturing unit 30 is bent along the first round surface 1237a of the guide structure 1231 even when the direction-changing protrusion 1234 is not present. However, since an end of the puncturing needle 31 is sharp, a locking phenomenon may occur. The direction-changing protrusion 1234 may address such a problem.

In the above, the first round surface 1237a may be formed to have any curvature that does not cause the end of the puncturing needle 31 to come into contact with the first round surface 1237a. However, to allow the puncturing needle 31 or the irrigation tube 60 to form a right angle with the sheath opening 1233 when exiting the sheath 123 via the sheath opening 1233, a front portion of the sheath space 1232 may be formed to be more round like a second round surface 1237b having a curvature greater than that of the first round surface 1237a. That is, when the puncturing unit 30 or the irrigation tube 60 is pushed in the state illustrated in FIG. 26C, as the puncturing unit 30 or the irrigation tube 60 is leaned toward the second round surface 1237b, the puncturing needle 31 or the irrigation tube 60 is bent further, and an end thereof forms a right angle with the sheath opening 1233. A vertical direction-changing protrusion (not illustrated) may be further provided at a proper position of the second round surface 1237b.

In order to minimize frictional resistance with the puncturing needle 31 and allow, as much as possible, the end of the puncturing needle 31 to be bent toward the sheath opening 1233 while not coming into contact with the guide structure 1231, a surface of the direction-changing protrusion 1234 that comes into contact with the puncturing needle 31 may be formed to be inclined by a predetermined angle. For example, the inclined surface of the direction-changing protrusion 1234 may be formed at an angle corresponding to an angle of inclination of the puncturing needle.

Also, it may be preferable that the direction-changing protrusion 1234 has a height that allows only a side surface of the puncturing needle 31 to come into contact with the direction-changing protrusion 1234 and does not allow a cutting edge portion of the puncturing needle 31 to come into contact with the direction-changing protrusion 1234. This is because, when the height of the direction-changing protrusion 1234 is high to an extent that the cutting edge portion of the puncturing needle 31 comes into contact with the direction-changing protrusion 1234, the cutting edge portion of the puncturing needle 31 may collide with the direction-changing protrusion 1234, and thus the locking phenomenon may occur.

The emergency treatment hole 1235 may be formed to pass through the sheath space 1232 from an upper portion of the guide structure 1231 so that, when the puncturing unit 30 or the irrigation tube 60 for puncturing or irrigation is locked to a wall of the sheath 123, i.e., an inner wall of the guide structure 1231, and thus is unable to smoothly move forward, a tool such as a wire is inserted through the emergency treatment hole 1235 so as to push the puncturing unit 30 or the irrigation tube 60 and change a direction of the puncturing needle 31.

The endoscope insertion passage 124 may be provided in the shape of a tunnel at an inner lower portion of the sheath 123 in the longitudinal direction in order to allow an endoscope 95 of the endoscope unit 90 to be mounted that is capable of monitoring puncturing of the posterior fontanelle 109 using the puncturing unit 30 or the state in which the irrigation tube 60 is located at the posterior fontanelle 109.

The endoscope insertion passage 124 is a portion inserted into nasal cavities while the endoscope 95 is mounted thereon and may be provided to extend a predetermined length forward from the front end of the main body 10.

The surgical procedure means insertion passage 125 may be provided in the shape of a tunnel at an inner upper portion of the sheath 123 in the longitudinal direction in order to allow the puncturing unit 30 configured to puncture the posterior fontanelle 109 and the irrigation tube 60 configured to irrigate the posterior fontanelle 109 to be mounted.

The surgical procedure means insertion passage 125 may be divided into a portion inserted into the nasal cavities and a portion exposed to the outside during a surgical procedure, and an inlet portion 1251, into which the cautery wire 32 of the puncturing unit 30 is inserted, may be provided at a rear end of the portion exposed to the outside. Here, the portion of the surgical procedure means insertion passage 125 that is inserted into the nasal cavities may be referred to as a portion provided by extending a predetermined length forward from the front end of the main body 10, and the portion of the surgical procedure means insertion passage 125 that is exposed to the outside may be referred to as a portion provided at the upper portion of the main body 10.

The cautery wire 32 of the puncturing unit 30 that is inserted into the surgical procedure means insertion passage 125 may come out to the outside via the inlet portion 1251 so as to be connected to the operation unit 40 or to be replaced. Therefore, the cautery wire 32 may be inserted from the rear end of the surgical procedure means insertion passage 125 into the front end thereof through the inlet portion 1251 of the surgical procedure means insertion passage 125. Here, the inlet portion 1251 may be provided at the rear end of the surgical procedure means insertion passage 125, be formed in the shape of a funnel having a cross-sectional area that gradually increases rearward, and form an inlet of the surgical procedure means insertion passage 125 for introducing the cautery wire 32. The inlet portion 1251 is formed as above in order to allow the cautery wire 32 to be easily introduced into the surgical procedure means insertion passage 125.

In the surgical procedure means insertion passage 125, a passage diameter of the portion exposed to the outside may be larger than a passage diameter of the portion inserted into the nasal cavities. This is because, although a passage of the portion inserted into the nasal cavities is formed of a minimum space in which the cautery wire 32 of the puncturing unit 30 and the irrigation tube 60 are mounted, a passage of the portion exposed to the outside requires, in addition to the above space, a space in which a cautery wire protection tube 322a, which is configured to surround the cautery wire 32 and the irrigation tube 60, is mounted. Thus, in the surgical procedure means insertion passage 125, a boundary between the portion inserted into the nasal cavities and the portion exposed to the outside may be curved.

A fixing clip 3221 configured to fix the cautery wire protection tube 322a mounted in the passage of the portion exposed to the outside may be further provided at the inlet portion 1251 of the surgical procedure means insertion passage 125.

FIGS. 27A to 27C are views for describing another embodiment of the first surgical procedure tube 13 in the maxillary sinus puncturing apparatus 2 according to the second embodiment of the present invention. FIG. 27A illustrates an initial state of the first surgical procedure tube 13 in which, together with the irrigation tube 60, the puncturing unit 30 is inserted into the surgical procedure means insertion passage 125 of the sheath 123; FIG. 27B illustrates a state in which the front end of the puncturing unit 30 is rotated, the puncturing unit 30 is moved forward toward a movable guide structure 1231a, and thus the puncturing needle 31 comes into contact with an inner surface of the movable guide structure 1231a and is guided toward the sheath opening 1233; and FIG. 27C illustrates a state in which, as the movable guide structure 1231a moves rearward due to an external force, the puncturing unit 30 is pushed, and thus the puncturing needle 31, which is in a vertical state, is guided toward the sheath opening 1233.

FIGS. 27A and 27B show the process before the sheath 123 is inserted into the nasal cavities, and FIG. 27C illustrates a state in which the sheath 123 is inserted into the nasal cavities and the sheath opening 1233 is located at a portion to be punctured of the posterior fontanelle 109.

Referring to FIG. 27, the sheath 123 includes the movable guide structure 1231a, the sheath space 1232, the sheath opening 1233, the direction-changing protrusion 1234, the emergency treatment hole 1235, the endoscope insertion passage 124, and the surgical procedure means insertion passage 125.

As compared with the configuration of the sheath 123 of FIG. 26, the configuration of the sheath 123 of FIG. 27 is different only in that the guide structure is configured to be movable, and the sheath space 1232, the sheath opening 1233, the direction-changing protrusion 1234, the emergency treatment hole 1235, the endoscope insertion passage 124, and the surgical procedure means insertion passage 125, which are the remaining elements, are the same. Accordingly, to avoid repetitive description here, detailed description of the same elements will be omitted, and only the movable guide structure 1231a, which is a different element, and changes that occur due to the movable guide structure 1231a will be described.

Generally, a certain amount of force should be applied to the puncturing needle 31 in order to allow the puncturing needle 31 to puncture the posterior fontanelle 109, pass through the posterior fontanelle 109 up to the irrigation tube 60, which has a larger diameter, and enter the maxillary sinus 100. However, in the case of the fixed guide structure 1231 illustrated in FIG. 26, since the puncturing needle 31 only relies on the force that moves the puncturing unit 30 forward, when a situation in which the force is not able to be transmitted to the puncturing needle 31 occurs, there is a possibility that the cautery wire 32 including the irrigation tube 60 may be bent in an unexpected direction.

As the movable guide structure 1231a moves rearward due to resistance that is generated when the puncturing needle 31 punctures the posterior fontanelle 109, passes through the posterior fontanelle 109 up to the irrigation tube 60, which has a larger diameter, and attempts to enter the maxillary sinus 100, the movable guide structure 1231a pushes the puncturing needle 31, and thus, a force of the movable guide structure 1231a is applied to the puncturing needle 31, in addition to the force that moves the puncturing unit 30 forward. Also, as the movable guide structure 1231a moves rearward, the movable guide structure 1231a causes the inlet of the sheath opening 1233, which is a passage through which the puncturing needle 31 passes, to become narrow. In this way, an occurrence of the situation in which the cautery wire 32 including the irrigation tube 60 is bent in an unexpected direction and thus a force is not able to be transmitted to the puncturing needle 31 may be prevented.

FIGS. 28A to 28C are views for describing still another embodiment of the first surgical procedure tube 13 in the maxillary sinus puncturing apparatus 2 according to the second embodiment of the present invention. FIG. 28A illustrates an initial state of the first surgical procedure tube 13 in which, together with the irrigation tube 60, the puncturing unit 30 is inserted into the surgical procedure means insertion passage 125 of the sheath 123; FIG. 28B illustrates a state in which the front end of the puncturing unit 30 is rotated, the puncturing unit 30 is moved forward toward the movable guide structure 1231a, and thus the puncturing needle 31 comes into contact with the inner surface of the movable guide structure 1231a and is guided toward the sheath opening 1233; and FIG. 28C illustrates a state in which, as the movable guide structure 1231a moves rearward due to an external force, the puncturing unit 30 is pushed, and thus the puncturing needle 31, which is in a vertical state, is guided toward the sheath opening 1233.

FIGS. 28A and 28B show the process before the sheath 123 is inserted into the nasal cavities, and FIG. 28C illustrates a state in which the sheath 123 is inserted into the nasal cavities and the sheath opening 1233 is located at a portion to be punctured of the posterior fontanelle 109.

Referring to FIG. 28, the sheath 123 includes the movable guide structure 1231a, the sheath space 1232, the sheath opening 1233, the direction-changing protrusion 1234, the emergency treatment hole 1235, the endoscope insertion passage 124, the surgical procedure means insertion passage 125, and a fixing key 1236.

As compared with the configuration of the sheath 123 of FIG. 27, the configuration of the sheath 123 of FIG. 28 is different only in that the fixing key 1236 is added, and the movable guide structure 1231a, the sheath space 1232, the sheath opening 1233, the direction-changing protrusion 1234, the emergency treatment hole 1235, the endoscope insertion passage 124, and the surgical procedure means insertion passage 125, which are the remaining elements, are the same. Accordingly, to avoid repetitive description here, detailed description of the same elements will be omitted, and only the fixing key 1236, which is a different element, and changes that occur due to the fixing key 1236 will be described.

The fixing key 1236 may be provided at an upper portion of the movable guide structure 1231a and may be a rotatable structure, so that the movable guide structure 1231a may maintain the state of being moved rearward due to the resistance that occurs when the movable guide structure 1231a attempts to enter the middle meatus 103.

The fixing key 1236 is tilted when the movable guide structure 1231a moves forward and then, when the movable guide structure 1231a moves rearward, the fixing key 1236 becomes vertical and corresponds to an upper surface of the sheath 123. When the sheath 123 is used in the middle meatus 103 in which the posterior fontanelle 109 is located, due to the anatomical structure in the narrow middle meatus 103, the upper surface of the sheath 123 receives pressure, and the fixing key 1236 is fixed such that it is not movable. Thus, even when a force is transmitted to the puncturing needle 31 in order to puncture the posterior fontanelle 109, the movable guide structure 1231a is not able to move forward.

When restricting the forward movement of the movable guide structure 1231a by the fixing key 1236, which is a rotatable structure, is not easy, a vertical structure such as a screw may be used in place of the fixing key 1236.

FIGS. 29A to 29C are views for describing the shape of the movable guide structure 1231a of FIGS. 27 and 28 and illustrate an inner portion of a front end of the movable guide structure 1231a, which is a plate-shaped structure.

FIG. 29A illustrates a front portion of the movable guide structure 1231a that corresponds to the handle. The front portion may be formed to be thick as a whole.

FIG. 29B illustrates a middle portion of the movable guide structure 1231a. The middle portion may be formed to be thin as a whole.

FIG. 29C illustrates a rear portion of the movable guide structure 1231a. The rear portion may be formed such that it is thin as the middle portion but has a lower end which is thick as the front portion.

The movable guide structure 1231a is formed in the above shape so that, while the puncturing needle 31 enters the front end of the sheath 123, the movable guide structure 1231a is moved forward to secure a space inside the sheath 123 and allow the puncturing needle 31 to move smoothly. That is, in order to facilitate the forward movement of the movable guide structure 1231a, the front portion corresponding to the handle may be formed to be thick, and a rear lower end portion may also be formed to be thick. This is to prevent the movable guide structure 1231a from falling out of the sheath 123. The movable guide structure 1231a may be manufactured with a material such as glass in order to block transfer of heat to the sheath 123, which may be manufactured with metal, during cauterization of the mucosa 108 using the puncturing needle 31.

The puncturing unit 30 may be provided in the surgical procedure means insertion passage 125 and punctures the posterior fontanelle 109. Since the posterior fontanelle 109 corresponds to a portion formed only of the mucosa 109 and not having the bones 107, efficiency of a surgical procedure may be maximized in the present invention by allowing the irrigation tube 60, which is configured to irrigate the maxillary sinus 100, to be installed by passing through only the mucosa 108.

The puncturing unit 30 may include the puncturing needle 31, the cautery wire 32, a puncturing electrocauterizer main body 33, a cautery activation button 34, a switch 35, and a socket 36.

As compared with the first embodiment, the puncturing unit 30 according to the second embodiment is the same or similar in terms of the configurations of the puncturing needle 31 and the cautery wire 32, is different in that the puncturing unit 30 is inserted and mounted in the surgical procedure means insertion passage 125, and further includes the puncturing electrocauterizer main body 33, the cautery activation button 34, the switch 35, and the socket 36. Accordingly, to avoid repetitive description here, detailed description on the puncturing needle 31 and the cautery wire 32 will be omitted, and only the puncturing electrocauterizer main body 33, the cautery activation button 34, the switch 35, and the socket 36 and changes that occur due to the elements will be described.

Each of the puncturing electrocauterizer main body 33, the cautery activation button 34, the switch 35, and the socket 36 may be provided in the main body 10.

The puncturing electrocauterizer main body 33 may be provided inside the main body handle 42 of the main body 10 and may have a battery embedded therein. The puncturing electrocauterizer main body 33 may be connected to the cautery wire 32 via a wire and allow electricity to be supplied to the puncturing needle 31 via the cautery wire 32.

The socket 36 may be further provided at the wire between the puncturing electrocauterizer main body 33 and the cautery wire 32 so as to connect or disconnect the wire. The socket 36 for the puncturing electrocauterizer main body may be connected to the socket 3211, which is provided at the locking protrusion 321, via an external wire.

Also, the switch 35 may be further provided between the puncturing electrocauterizer main body 33 and the cautery wire 32 so as to turn electricity for driving the puncturing electrocauterizer main body 33 on or off. The switch 35 may include an LED-on-lap function.

The cautery activation button 34 may be connected to the puncturing electrocauterizer main body 33 via a wire so as to activate a cauterizing function of the puncturing electrocauterizer main body 33. The cautery activation button 34 may be provided at the front end of the main body 10 so that the cautery activation button 34 may be easily manipulated by the operator.

Meanwhile, in the cautery wire 32, a portion which extends from the inlet portion 1251 of the surgical procedure means insertion passage 125 to the moving block 410 may be exposed to the outside, and the exposed cautery wire 32 may be bent when the moving block 410 moves forward. Accordingly, the cautery wire protection tube 322a may be further provided in order to protect the exposed cautery wire 32.

As compared with the cautery wire protection tube 322 according to the first embodiment described above, the cautery wire protection tube 322a according to the second embodiment may extend further up to the surgical procedure means insertion passage 125 at the portion exposed to the outside from the upper end of the main body 10 so that the cautery wire protection tube 322a is also used for the purpose of sterilizing the apparatus in addition to being used for the purpose of preventing the cautery wire 32 from being bent.

That is, the cautery wire protection tube 322a according to the second embodiment may have one end inserted and fixed to a through-hole 4100a of the moving block 410 and may extend to the boundary between the portion of the surgical procedure means insertion passage 125 that is inserted into the nasal cavities and the portion thereof exposed to the outside. In this way, the cautery wire protection tube 322a may be used for sterilizing the apparatus as well as for preventing the cautery wire 32 from being bent when the moving block 410 moves forward. The cautery wire protection tube 322a which is inserted and mounted in the passage of the portion exposed to the outside may be fixed by the fixing clip 3221 provided at the inlet portion 1251 of the surgical procedure means insertion passage 125.

The maxillary sinus puncturing apparatus 2 should be thoroughly sterilized since it is used in the human body. When the cautery wire 32 is used for a patient and then removed, the surgical procedure means insertion passage 125 becomes unclean. Sterilization is performed on the basis of principles for sterilizing endoscopes such as a gastroscope. Since, when a liquid (sterilant) enters various structures provided in the main body 10 such as the cautery activation button 34 or an endoscope head 94 provided at the front portion of the main body 10, there is concern about damaging the structures, the maxillary sinus puncturing apparatus 2 should be sterilized by putting only up to the sheath 123, which is ahead of the cautery activation button 34 or the endoscope head 94 provided at the front portion of the main body 10, into the sterilant. Generally, the maxillary sinus puncturing apparatus 2 which stands upright is put into a container containing a sterilant so as to be sterilized. For the rear portion of the maxillary sinus puncturing apparatus 2 that does not come into contact with the sterilant, the sterilant is injected through the inlet portion 1251 of the surgical procedure means insertion passage 125 for irrigation and sterilization, and, to better ensure disinfection, the cautery wire protection tube 322a which is completely sterilized may be additionally inserted. Since the irrigation tube 60 is inserted into the completely sterilized cautery wire protection tube 322a, the irrigation tube 60 may be used without the risk of injection.

The irrigation tube 60 may be mounted in the surgical procedure means insertion passage 125 together with the puncturing unit 30 configured to puncture the posterior fontanelle 109.

The irrigation tube 60 may be provided to be fitted to the cautery wire 32 in the surgical procedure means insertion passage 125 and may be installed at the posterior fontanelle 109 punctured by the puncturing needle 31. The irrigation tube 60 is a configuration for allowing the inner portion and the outer portion of the maxillary sinus 100 to communicate. After the posterior fontanelle 109 is punctured by the puncturing unit 30, the irrigation tube 60 may remain installed in the punctured hole. The irrigation tube 60 is installed in order to irrigate the maxillary sinus 100 and may be used for the purpose of administering drugs into the maxillary sinus 100 as necessary.

In the irrigation tube 60, a towline 61 may be further provided at an inlet through which drugs are administered. The towline 61 will be described below with reference to FIGS. 41 and 42.

FIGS. 41A to 41C are views for describing the irrigation tube 60. FIG. 41A illustrates a state before the trigger 413a is pulled; FIG. 41B illustrates a state after the trigger 413a is pulled; and FIG. 41C illustrates an embodiment of the locking protrusion 321 of FIG. 5 that is suitable for the irrigation tube 60 in which the towline 61 is provided. FIG. 42 is a front cross-sectional view of a face illustrating a state in which the irrigation tube 60 is inserted into the maxillary sinus 100. The towline 60 may not only serve as a handle when the irrigation tube 60 is pulled but also serve to fix the irrigation tube 60 as illustrated in FIG. 42 so that the irrigation tube 60 is not pushed rearward while it is inserted into the maxillary sinus 100.

The towline 61 may be provided at an inlet portion of the irrigation tube 60. The towline 61 may be an upper portion which is left after a lower portion is removed after horizontally cutting the irrigation tube 60. Alternatively, the towline 61 may be provided by being separately attached to the inlet of the irrigation tube 60.

After the irrigation tube 60, the puncturing needle 31, and the cautery wire 32 are inserted into the maxillary sinus 100, only the irrigation tube 60 may be left in the maxillary sinus 100, and the puncturing needle 31 and the cautery wire 32 may be removed. For this, while the irrigation tube 60 is fixed, the cautery wire 32, the puncturing needle 31, and the locking protrusion 321 connected thereto are moved backward. When the trigger 413a is pulled from the state illustrated in FIG. 41A, and the moving block 410 is moved forward as in the state illustrated in FIG. 41B, the irrigation tube 60, the cautery wire 32, and the puncturing needle 31 enter the maxillary sinus 100. In this state, the fixing member 4102 is released such that the through-hole 4100a is exposed to the upper side, and the towline 61 is pulled upward and forward such that the irrigation tube 60 is not pushed rearward. Then, when the trigger 413a is slowly released, the moving block 410 moves rearward due to a force of the spring 432, and the locking protrusion 321, the cautery wire 32, and the puncturing needle 31 which are inside the irrigation tube 60 also move rearward together.

In order to prevent the irrigation tube 60 from being pushed rearward while it is inserted into the maxillary sinus 100, the towline 61 is pushed upward and rearward. For this, a groove 3212 which is open upward may be formed in the locking protrusion 321, and, in this way, the towline 61 may move upward and forward through the groove 3212.

The endoscope unit 90 may be provided in the endoscope insertion passage 124 and the main body 10 so as to monitor puncturing of the posterior fontanelle 109 using the puncturing unit 30 or the state in which the irrigation tube 60 is located at the posterior fontanelle 109. The endoscope unit 90 may be configured to include an endoscope camera main body 91, an endoscope camera monitor 92, an endoscope camera head 93, the endoscope head 94, the endoscope 95, an endoscope window 96, and a small LED light source device or a light source cable 97.

The endoscope camera main body 91 may be provided at the rear end of the main body 10 and have a battery embedded therein. The endoscope camera main body 91 may supply electricity to the endoscope camera monitor 92, the endoscope camera head 93, the endoscope head 94, the endoscope 95, and the like.

The endoscope camera main body 91 may be manufactured so that an upper end surface thereof is placed on a straight line with the upper end surface of the main body 10. This is to prevent interference when the cautery wire protection tube 322a, which is manufactured in a lengthy form, is inserted into the surgical procedure means insertion passage 125 via the through-hole 4100a.

The endoscope camera monitor 92 may be mounted on one end of the endoscope camera main body 91 and may be provided to be tilted vertically and horizontally to allow the screen to be well-visible.

The endoscope camera head 93 may include an image sensor and convert an image into an electrical signal. The endoscope camera head 93 may be connected to the endoscope camera main body 91 and provided at the front end of the main body 10.

The endoscope head 94 is a portion where optical fibers and a lens coupled thereto are present. The endoscope head 94 may be connected to the endoscope camera head 93 and provided at the front end of the main body 10.

The endoscope 95 may be connected to the endoscope head 94 and mounted inside the endoscope insertion passage 124. When the endoscope 95 is used at 30° or 45° instead of 0°, front blocking due to the sheath 123 may be avoided.

The endoscope window 96 may be connected to a front end of the endoscope 95. The endoscope window 96 may be provided to be exposed to the outside from the front end of the endoscope insertion passage 124 so that a field of view is easily secured when the endoscope 95 monitors puncturing of the posterior fontanelle 109 using the puncturing unit 30 or the state in which the irrigation tube 60 is located at the posterior fontanelle 109.

A groove 961 having a concave shape may be formed at one side surface of the endoscope window 96 as illustrated in FIG. 30. Since the puncturing needle 31, the cautery wire 32, and the irrigation tube 60 may enter up to the one side surface, when the one side surface of the endoscope window 96 is formed to have the concave groove 961 rather than being formed in a planar shape, the cautery wire 32 and the irrigation tube 60 are allowed to be bent further, and the puncturing needle 31 is allowed to perform puncturing in a state closer to being vertical.

The small LED light source device or the light source cable 97 is a portion configured to transmit light to the endoscope 95 and may be provided at the front end of the main body 10. Light may be transmitted through the cable while a light source device main body (not illustrated) is disposed outside, or the small LED light source device may be used by being attached to the main body 10.

In the endoscope unit 90, since, when an electronic endoscope is used instead of a fiber optic endoscope as the endoscope 95, the image sensor is disposed right behind an objective lens and only optical fibers and an image line pass through the endoscope insertion passage 124, which is lengthy, the endoscope insertion passage 124 may be slightly curved instead of having a linear structure as a whole. Therefore, the curve, which is generated at the boundary between the portion of the surgical procedure means insertion passage 125 that is inserted into the nasal cavities and the portion thereof exposed to the outside, may be made almost straight.

In the above, the puncturing needle 31 turns toward the maxillary sinus 100 even when, as illustrated in FIG. 31, the first surgical procedure tube 13 is inserted into the middle meatus 103 in form “a” and then slightly rotates as in form “c” instead of rotating at a right angle as in form “b.” Therefore, the apparatus can puncture the posterior fontanelle 109 without difficulty even in the narrow middle meatus 103.

The operation unit 40 is provided in the main body 10 and moves the puncturing unit 30 of the first surgical procedure tube 13 toward the posterior fontanelle 109. The operation unit 40 is a configuration that allows the puncturing needle 31 of the puncturing unit 30 to come into contact with the posterior fontanelle 109 so that puncturing of the posterior fontanelle 109 is realized. Specifically, the operation unit 40 may allow the cautery wire 32 to move forward inside the surgical procedure means insertion passage 125.

The operation unit 40 may include the moving unit 41, which includes the moving block 410, the rail 412, and the trigger 413, the main body handle 42, and the moving block position adjuster 43.

As compared with the configuration of the operation unit 40 according to the first embodiment described above, the operation unit 40 according to the second embodiment is different in that the through-hole 4100a passing through both side surfaces of the moving block 410 is formed instead of the fixing groove 4100 which passes through only one side surface of the moving block 410 such that the other side surface of the moving block 410 is blocked and is different in terms of the configuration of the trigger 413a. Accordingly, to avoid repetitive description here, detailed description of the same elements will be omitted, and only the different elements will be described.

To allow the cautery wire protection tube 322a, which is manufactured to be lengthy, to be easily inserted into the surgical procedure means insertion passage 125 from the outside, the through-hole 4100a is manufactured to pass through both side surfaces of the moving block 410.

The trigger 413a may move back and forth along the first passage 10a of the main body 10 and be formed to have a plate-shaped structure. Since the trigger 413a is formed to have the plate-shaped structure, the trigger 413a may interfere with the main body handle 42 when the trigger 413a moves backward. In order to prevent the interference, an accommodating groove 421 in which a rear end of the trigger 413a may be accommodated may be formed in a front portion of the main body handle 42.

The ultrasonic probing unit 20 is a device configured to detect a punctured position of the posterior fontanelle 109 using ultrasonic waves. The ultrasonic probing unit 20 may include the ultrasonic main body 21 which is provided at a rear end of the probing tube 11, the ultrasonic probe 22 provided at a front end of the probing tube 11, and a coaxial lead wire 23 provided inside the probing tube 11. The ultrasonic probing unit 20 which is manufactured as the detachable type model that is detachable from the main body 10 may further include the probing unit handle 26 provided at a front portion of the ultrasonic main body 21 and the probing monitor 27 provided at a rear portion of the ultrasonic main body 21.

As compared with the first embodiment, the ultrasonic probing unit 20 according to the second embodiment is different in that it is configured to be coupled to the marking unit 28, and basic configurations and functions of the ultrasonic probing unit 20 according to the second embodiment are the same as or similar to the first embodiment. Therefore, to avoid repetitive description here, detailed description of the basic configurations and functions will be omitted. Configurations of the ultrasonic probing unit 20 according to the second embodiment that are different from the first embodiment should be understood from the following description of the marking unit 28.

The marking unit 28 may mark a position to be punctured on the mucosa 108 at a portion expected to be the posterior fontanelle 109 in the middle meatus 103 detected by the ultrasonic probing unit 20 and may be provided to be coupled to the ultrasonic probing unit 20. Hereinafter, the marking unit 28 will be described in detail with reference to FIGS. 35 to 40.

FIG. 36 is a cross-sectional view taken along line X-X′ of FIG. 35 that is for describing a first embodiment of the marking unit 28 in the maxillary sinus puncturing apparatus 2 according to the second embodiment of the present invention. The marking unit 28 includes a marking electrocauterizer main body 2811, a marking activation button 2812, a first wire 2813, a needle passage 2814, and a first marking needle 2815.

The marking electrocauterizer main body 2811 may be provided between the ultrasonic main body 21 and the probing tube 11 and have a battery embedded therein. The marking electrocauterizer main body 2811 may supply electricity to the first marking needle 2815 via the first wire 2813.

The marking activation button 2812 may be connected to the marking electrocauterizer main body 2811 via a wire (not illustrated) and may activate a marking function of the marking electrocauterizer main body 2811. The marking activation button 2812 may be provided at a portion of the marking electrocauterizer main body 2811 so as to be easily manipulated by the operator.

The first wire 2813 may extend from the marking electrocauterizer main body 2811 to the needle passage 2814 and may be provided inside the probing tube 11. The first wire 2813 allows electricity to be supplied from the marking electrocauterizer main body 2811 to the first marking needle 2815.

The needle passage 2814 may be provided at a central portion or an edge portion of the ultrasonic probe 22 so that the first marking needle 2815 may be accommodated in the needle passage 2814. The needle passage 2814 may have a structure which is open toward the liquid structure 222. The needle passage 2814 may be manufactured with an insulator since the electricity flowing through the first marking needle 2815 may cause damage to the ultrasonic probe 22.

The first marking needle 2815 may be provided inside the needle passage 2814. The first marking needle 2815 may be connected to the first wire 2813, receive electricity from the marking electrocauterizer main body 2811, and burn the mucosa 108 at a portion expected to be the posterior fontanelle 109 by electrocauterization so as to mark a position to be punctured.

The marking unit 28 configured as above burns the mucosa 108 of the posterior fontanelle 109 by electrocauterization and leaves a mark on the mucosa 108. That is, the marking unit 28 uses a burning method instead of a puncturing method. Therefore, an electrocauterization method applied to the first marking needle 2815 is in a coagulation mode instead of a cutting mode. Also, since the electricity flowing through the first marking needle 2815 may cause damage to the ultrasonic probe 22, the needle passage 2814 may be manufactured with an insulator and manufactured so that, when the marking activation button 2812 is pressed, electricity is automatically supplied only for short time and then cut off.

Since, in the posterior fontanelle 109, a portion ideal for puncturing is extremely small, it is preferable that an accurate position be marked after ultrasonic probing is performed using the ultrasonic probe 22 of the ultrasonic probing unit 20. For this, the needle passage 2814 which accommodates the first marking needle 2815 is disposed at the center of the ultrasonic probe 22 so that a portion to be punctured may be marked at the center of the ultrasonic probe 22. The first marking needle 2815 is manufactured to be replaceable in order to prevent another patient from being infected with a disease that the patient has.

FIG. 37 is a cross-sectional view taken along line X-X′ of FIG. 35 that is for describing a second embodiment of the marking unit 28 in the maxillary sinus puncturing apparatus 2 according to the second embodiment of the present invention. The marking unit 28 includes a first fluid pipe 2821, a first cylinder tube 2822, a push-pull means 2823, a first piston 2824, and a second marking needle 2825.

The first fluid pipe 2821 is a passage through which a fluid is injected into or discharged from the push-pull means 2823. The first fluid pipe 2821 may be provided in the longitudinal direction inside the probing tube 11 and may pass through the ultrasonic probe 22 and longitudinally extend to the first cylinder tube 2822, which will be described below. One end of the first fluid pipe 2821 is connected to the push-pull means 2823, which will be described below, that is provided at a portion of the ultrasonic probe 22, and the other end of the first fluid pipe 2821 is exposed to the outside.

A fluid injection/discharge means (not illustrated) such as a syringe is connected to the other end of the first fluid pipe 2821 that is exposed to the outside, and the first fluid pipe 2821 injects or discharges a fluid such as air into or from the push-pull means 2823, thereby allowing the second marking needle 2825, which will be described below, to mark a position to be punctured on the mucosa 108 of the posterior fontanelle 109.

The first cylinder tube 2822 may be provided at the central portion or the edge portion of the ultrasonic probe 22 and accommodate the push-pull means 2823, the first piston 2824, and the second marking needle 2825 and may have the structure which is open toward the liquid structure 222.

An upper inner surface of the first cylinder tube 2822 may be formed to be smooth so that the push-pull means 2823, which will be described below, may freely contract or expand. In order to allow the first piston 2824, which will be described below, to move upward and downward uniformly, as illustrated in FIG. 38, a plurality of guide bars 2827 may be provided at a lower inner surface of the first cylinder tube 2822. Also, in order to prevent the first piston 2824, which will be described below, from deviating from the first cylinder tube 2822, one or more stoppers 2826 may be provided at the lower inner surface of the first cylinder tube 2822.

The push-pull means 2823 may be provided to be connected from an inner upper portion of the first cylinder tube 2822 to one end of the first fluid pipe 2821. The first piston 2824, which will be described below, may be connected to the push-pull means 2823. The push-pull means 2823 may be manufactured as a balloon type or a bellows type.

The push-pull means 2823 may expand or contract due to the fluid injected into or discharged from the push-pull means 2823 through the first fluid pipe 2821. Due to the expansion and contraction, the first piston 2824, which will be described below, may move upward and downward, and the second marking needle 2825, which will be described below, is allowed to mark a position to be punctured on the mucosa 108 of the posterior fontanelle 109.

The first piston 2824 may be provided to be connected from the inner lower portion of the first cylinder tube 2822 to the push-pull means 2823. The second marking needle 2825, which will be described below, may be connected to the first piston 2824.

The first piston 2824 may have a shape corresponding to an inner peripheral surface of the first cylinder tube 2822 as a whole. In order to allow the first piston 2824 to uniformly move when moving upward and downward due to the contraction or expansion of the push-pull means 2823, as illustrated in FIG. 38, a plurality of guide grooves 2828 corresponding to the plurality of guide bars 2827 may be formed in an outer peripheral surface of the first piston 2824.

The second marking needle 2825 may be provided to be connected from the inner lower portion of the first cylinder tube 2822 to the first piston 2824.

As the push-pull means 2823 expands or contracts due to the fluid injected into or discharged from the push-pull means 2823 through the first fluid pipe 2821, the first piston 2824 moves upward and downward. Due to the upward and downward movement of the first piston 2824, the second marking needle 2825 may leave a scratch on the mucosa 108 of the posterior fontanelle 109 and thus mark a position to be punctured.

The second marking needle 2825 may be manufactured to be replaceable in order to prevent another patient from being infected with a disease that the patient has.

FIGS. 39A to 39C are cross-sectional views taken along line X-X′ of FIG. 35 that are for describing a third embodiment of the marking unit 28 in the maxillary sinus puncturing apparatus 2 according to the second embodiment of the present invention. FIG. 39A illustrates a preparation state; FIG. 39B illustrates a state in which a position to be punctured is marked or directly punctured; and FIG. 39C is an enlarged view for describing an airtight member. The marking unit 28 includes a second fluid pipe 2831, a second cylinder tube 2832, a second piston 2833, a tip 2834, a second wire 2835, and an airtight member 2836.

The second fluid pipe 2831 is a passage configured to inject or discharge a fluid into or from the second cylinder tube 2832, which will be described below. The second fluid pipe 2831 may be provided in the longitudinal direction outside the probing tube 11 and may pass through the ultrasonic probe 22 and longitudinally extend to the second cylinder tube 2832, which will be described below. One end of the second fluid pipe 2831 is connected to the second cylinder tube 2832, which will be described below, that is provided at a portion of the ultrasonic probe 22, and the other end of the second fluid pipe 2831 is exposed to the outside.

A fluid injection/discharge means (not illustrated) such as a syringe is connected to the other end of the second fluid pipe 2831 that is exposed to the outside, and the second fluid pipe 2831 injects or discharges a fluid such as air into or from the second cylinder tube 2832, which will be described below, thereby allowing the tip 2834, which will be described below, to mark a position to be punctured on the mucosa 108 of the posterior fontanelle 109 or to directly puncture the mucosa 108 of the posterior fontanelle 109.

The second cylinder tube 2832 may be provided at the central portion or the edge portion of the ultrasonic probe 22 and accommodate the second piston 2833 and may have a structure which is open toward the liquid structure 222. The airtight member 2836, which will be described below, may be provided at a lower portion of the second cylinder tube 2832.

The second piston 2833 may be accommodated inside the second cylinder tube 2832 and connected to the second wire 2835, which will be described below. The tip 2834, which will be described below, that is capable of marking a position to be punctured on the mucosa 108 of the posterior fontanelle 109 or directly puncturing the mucosa 108 of the posterior fontanelle 109 may be provided at the second piston 2833. The airtight member 2836, which will be described below, may be connected to a lower inner peripheral surface of the second piston 2833.

The second piston 2833 may move upward and downward due to the fluid injected into or discharged from the second piston 2833 through the second fluid pipe 2831, thereby allowing the tip 2834, which will be described below, to mark a position to be punctured on the mucosa 108 of the posterior fontanelle 109 or to directly puncture the mucosa 108 of the posterior fontanelle 109.

The tip 2834 may be provided at a lower end of the second piston 2833, and electricity may be supplied to the tip 2834 via the second wire 2835, which will be described below. The tip 2834 may be integrated with the second piston 2833 or detachable therefrom. The tip 2834 may be formed of a resistance material that generates heat when electricity is applied thereto.

The tip 2834 may mark a position to be punctured on the mucosa 108 of the posterior fontanelle 109 or directly puncture the mucosa 108 of the posterior fontanelle 109 by using an electrocauterization method.

The second wire 2835 may extend from an electricity supply source to the second cylinder tube 2832 so as to supply electricity to the tip 2834 and may be provided inside the second fluid pipe 2831.

Specifically, the second wire 2835 may extend from the electricity supply source to the second piston 2833 or the tip 2834 accommodated in the second cylinder tube 2832. When the second piston 2833 is a conductor, the second wire 2835 may be connected to the second pistons 2833 without extending to the tip 2834. The electricity supply source may be separately provided outside or may be a battery embedded in the marking electrocauterizer main body 2811.

In order to prevent the fluid injected through the second fluid pipe 2831 from leaking to the outside, the airtight member 2836 may have one end connected to the second cylinder tube 2832 and the other end connected to a lower portion of the second piston 2833. The airtight member 2836 may be formed with a flexible material in order to allow the second piston 2833 to freely move upward and downward.

The airtight member 2836 may also serve to prevent the second piston 2833 from deviating from the second cylinder tube 2832.

The marking unit 28 according to the third embodiment configured as above allows the puncturing electrocauterizer main body 33 to be removed from the puncturing unit 30. In this case, since the cautery wire 32 may be replaced with an ordinary wire, manufacturing of the maxillary sinus puncturing apparatus 2 is facilitated. When the mucosa 108 of the posterior fontanelle 109 is marked and then punctured by the tip 2834, even when the mucosa 108 of the posterior fontanelle 109 is not completely perforated, since the puncturing needle 31 included in the puncturing unit 30 is sharp, the mucosa 108 of the posterior fontanelle 109 may be easily punctured just by a physical force of pulling the trigger 413a, without using electrocauterization. Puncturing the mucosa 108 of the posterior fontanelle 109 by a physical force using the puncturing needle 31 does not cause any problem since the mucosa 108 becomes thinner due to the tip 2834 and bleeding is prevented.

Generally, in order to prevent fogging during a surgical procedure using an endoscope, an anti-fogging agent is used at the foremost portion of the endoscope 95, i.e., a lens portion of the endoscope 95. Since some anti-fogging agents are flammable, an accident may occur when electrocauterization is performed while using a flammable anti-fogging agent in the puncturing unit 30 in which the endoscope 95 is embedded. In the marking unit 28 according to the third embodiment described above, the mucosa 108 of the posterior fontanelle 109 is allowed to be directly punctured by the tip 2834. In this way, it is possible to prevent accidents even when an anti-fogging agent is used for the endoscope 95.

Also, since the puncturing unit 30 does not use a cauterization method for puncturing, the puncturing electrocauterizer main body 33 which is provided at the main body handle 42 and has a battery embedded therein may be removed, and a battery embedded in the endoscope camera main body 91 may be located in the main body handle 42. In this way, the size of the endoscope camera main body 91 may be reduced.

The marking unit 28 according to the third embodiment described above may further include the marking electrocauterizer main body 2811 and the marking activation button 2812 of the marking unit 28 according to the first embodiment. Here, the marking electrocauterizer main body 2811 may supply electricity to the tip 2834 via the second wire 2835, and the marking activation button 2812 may activate the marking function of the marking electrocauterizer main body 2811.

FIG. 40 is a cross-sectional view taken along line X-X′ of FIG. 35 that is for describing a fourth embodiment of the marking unit 28 in the maxillary sinus puncturing apparatus 2 according to the second embodiment of the present invention. The marking unit 28 includes the second fluid pipe 2831, the second cylinder tube 2832, the second piston 2833, the tip 2834, the second wire 2835, the airtight member 2836, and a surgical procedure means insertion passage 125a.

The second fluid pipe 2831, the second cylinder tube 2832, the second piston 2833, the tip 2834, the second wire 2835, and the airtight member 2836, which are the configurations of the marking unit 28 according to the fourth embodiment, are the same as or similar to the configurations of the marking unit 28 according to the third embodiment described above. Therefore, to avoid repetitive description here, detailed description thereof will be omitted, and only the surgical procedure means insertion passage 125a, which is a different element, and changes that occur due to the surgical procedure means insertion passage 125a will be described.

The surgical procedure means insertion passage 125a may be provided in the shape of a tunnel in the longitudinal direction at the inner upper portion of the probing tube 11 so that the puncturing unit 30 configured to puncture the posterior fontanelle 109 and the irrigation tube 60 configured to irrigate the posterior fontanelle 109 may be mounted in the surgical procedure means insertion passage 125a.

The marking units 28 according to the third and fourth embodiments which have been described above with reference to FIGS. 39 and 40 require the second piston 2833 and another component related thereto as configurations for employing an electrocauterization method. The marking units 28 according to the third and fourth embodiments may be configured to employ a laser method other than the electrocauterization method. Although a configuration for employing the laser method is not illustrated in the drawings, when laser is used, only a passage through which a laser beam passes, i.e., a structure such as a cylinder, is required. In this case, since the second piston 2833 or a component related thereto may be omitted, the ultrasonic probing unit 20 including the marking unit 28 can be made simple.

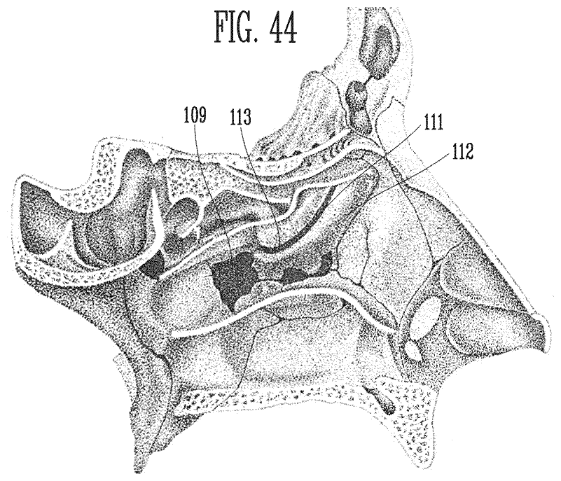

In the above-described embodiments, the ultrasonic probing unit 20 using ultrasonic waves has been described as a device for detecting a punctured position of the posterior fontanelle 109. However, the posterior fontanelle 109 may be detected using light, and this will be described below with reference to FIGS. 43 and 44.

FIG. 43 is a view illustrating a light probing unit configured to detect a posterior fontanelle using light in the maxillary sinus puncturing apparatus according to the present invention, and FIG. 44 is a left cross-sectional view of a face for describing a method of detecting a posterior fontanelle using the light probing unit of FIG. 43.

Referring to FIG. 43, a light probing unit 20a is a device configured to detect a punctured position of the posterior fontanelle 109. Unlike the ultrasonic probing unit 20 described above, the light probing unit 20a may be configured to detect a punctured position of the posterior fontanelle 109 using light.

The light probing unit 20a may include a light probing main body 21a, a light emitting unit 29a, and a light receiving unit 29b. The light probing unit 20a which is manufactured as a detachable type model that is detachable from the main body 10 may further include first and second handles 26a and 26b, which are provided at a front portion of the light probing main body 21a, and a light probing monitor 27a which is provided at a rear portion of the light probing main body 21a.

The light probing main body 21a may generate an electrical signal for generating light.

The light emitting unit 29a may emit light due to the electrical signal from the light probing main body 21a and may be configured to include a first probing tube 11a, a first light probe 22a, and a first handle 26a. When the punctured position of the posterior fontanelle 109 is being detected, the light emitting unit 29a allows light to be emitted from a natural ostium 111, which is a portion of an infundibulum 113 that is recessed inward.

Basic configurations of the first probing tube 11a are the same as or similar to those of the probing tube 11 described above. Therefore, to avoid repetitive description here, detailed description of the basic configurations of the first probing unit 11a will be omitted. The light probing main body 21a may be connected to a rear end of the first probing tube 11a, and the first light probe 22a may be connected to a front end of the first probing tube 11a.

The first light probe 22a may be provided at the front end of the first probing tube 11a, may receive the electrical signal from the light probing main body 21a and emit light, and may be a light emitting device. When the punctured position of the posterior fontanelle 109 is being detected, the first light probe 22a may be located in the natural ostium 111, which is the portion of the infundibulum 113 that is recessed inward.

The first handle 26a may be provided at the first probing tube 11a which is in front of the light probing main body 21a and may adjust a position of the first light probe 22a when the punctured position of the posterior fontanelle 109 is being detected.

The light receiving unit 29b may sense light emitted from the first light probe 22a and transmit the sensed light to the light probing main body 21a. The light receiving unit 29b may be configured to include a second probing tube 11b, a second light probe 22b, and a second handle 26b. The light receiving unit 29b may receive light from the posterior fontanelle 109 when the punctured position of the posterior fontanelle 109 is being detected. The light receiving unit 29b may be configured to be coupled to the marking unit 28. This may be understood from the configuration in which the ultrasonic probing unit 20 and the marking unit 28 are coupled to each other.

Since basic configurations of the second probing tube 11b are the same as or similar to those of the probing tube 11 described above, in order to avoid repetitive description here, detailed description of the basic configurations of the second probing tube 11b will be omitted. The light probing main body 21a may be connected to a rear end of the second probing tube 11b, and the second light probe 22b may be connected to a front end of the second probing tube 11b.

The second light probe 22b may be provided at the front end of the second probing tube 11b, may sense light emitted from the first light probe 22a and transmit the sensed light to the light probing main body 21a, and may be a light receiving device. When the punctured position of the posterior fontanelle 109 is being detected, the second light probe 22b may be located in the posterior fontanelle 109.

The second handle 26b may be provided at the second probing tube 11b which is in front of the light probing main body 21a. The second handle 26b may adjust a position of the second light probe 22b when the punctured position of the posterior fontanelle 109 is being detected.

The light probing monitor 27a may be provided at a rear portion of the light probing main body 21a and may show information on light transmitted from the second light probe 22b by using an image.

A method of detecting the posterior fontanelle 109 using the light probing unit 20a will be described below with reference to FIG. 44.

Anatomically, there is one passage entering a maxillary sinus inside a nose. This passage is the natural ostium 111. It is difficult to approach the natural ostium 111 since the natural ostium 111 is occluded by bone referred to as an uncinate process 112. However, when light is transmitted to the maxillary sinus through the natural ostium 111 so that the light is visible through the posterior fontanelle 109 which is formed only of the mucosa without bones, the posterior fontanelle 109 may be identified.

The natural ostium 111 is located in a portion referred to as the infundibulum 113 that is recessed inward like a furrow. That is, the natural ostium 111 is a portion occluded by the uncinate process 112. Therefore, the first light probe 22a of the light emitting unit 29a is located in the natural ostium 111 so that the first light probe 22a emits light, the second light probe 22b of the light receiving unit 29b is located in the posterior fontanelle 109 so that the second light probe 22b senses the light emitted from the first light probe 22a, information on the sensed light is transmitted to the light probing main body 21a, and the information on the sensed light is shown on the light probing monitor 27a by using an image. In this way, the punctured position of the posterior fontanelle 109 may be identified. That is, the light that has entered the maxillary sinus through the natural ostium 111 may be visible through the posterior fontanelle 109 where there are no bones.

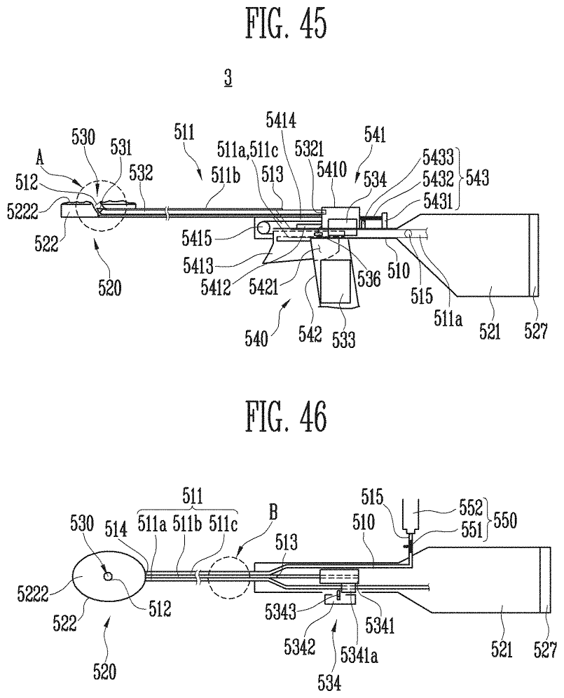

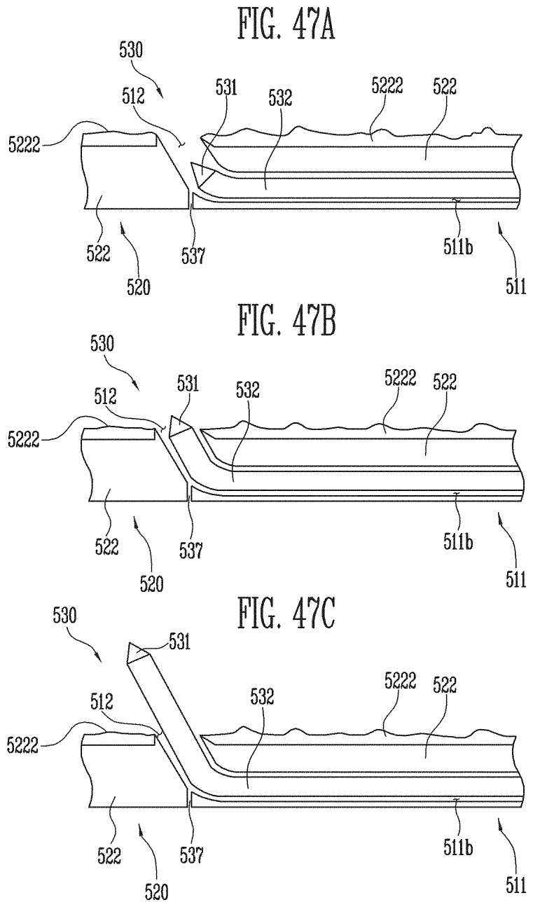

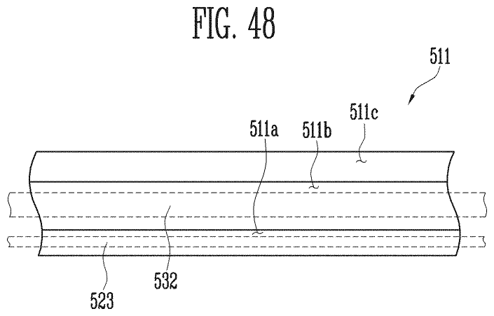

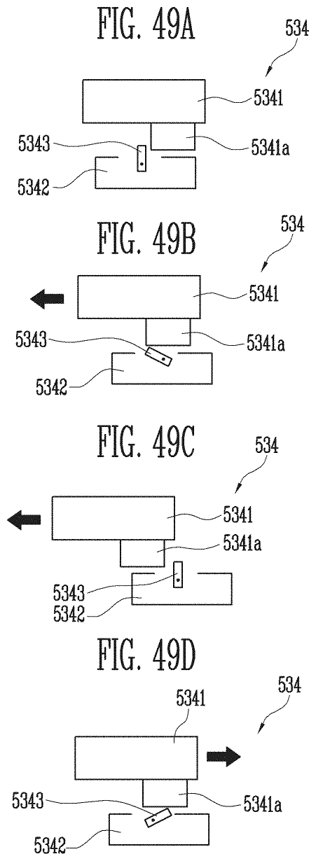

FIG. 45 is a bottom view of a maxillary sinus puncturing apparatus according to a third embodiment of the present invention; FIG. 46 is a side view of the maxillary sinus puncturing apparatus according to the third embodiment of the present invention; FIGS. 47A to 47C are enlarged views of portion A of FIG. 45; FIG. 48 is an enlarged view of portion B of FIG. 46; and FIGS. 49A to 49D are views for describing a cautery activation switch.

Referring to FIGS. 45 to 49, a maxillary sinus puncturing apparatus 3 according to the third embodiment of the present invention includes a main body 510, a second surgical procedure tube 511, an ultrasonic probing unit 520, a marking/puncturing unit 530, and an operation unit 540.

The main body 510 is a portion gripped by the operator. While gripping the main body 510, the operator may detect the posterior fontanelle 109 using the ultrasonic probing unit 520, which will be described below, mark a position to be punctured on the mucosa 108 at a portion expected to be the posterior fontanelle 109 in the middle meatus 103 that is detected by the ultrasonic probing unit 520 by using the marking/puncturing unit 530, which will be described below, and then puncture the maxillary sinus 100.

The main body 510 may be manufactured in various shapes to allow an index finger and a middle finger of the operator to perform different tasks while the operator is gripping the main body 10 and, at the same time, to facilitate securing a field of view during a surgical procedure. A direction of insertion into the middle meatus 103 through a nostril may be defined as the front, and the opposite direction may be defined as the rear. Here, the second surgical procedure tube 511 may be provided at a front portion of the main body 510. The second surgical procedure tube 511 may be configured to include a probing tube 511a, a marking/puncturing tube 511b, and a liquid tube 511c.

The main body 510 of the present embodiment may seem to be similar with the main body 10 in which the puncturing unit 30 is provided according to the first and second embodiments described above. However, taking into consideration that the ultrasonic probing unit 520 is provided in the main body 510 of the embodiment together with the marking/puncturing unit 530 and that the ultrasonic probe 522 has an oval surface in the ultrasonic probing unit 520, during actual use of the main body 510, a main body handle 542 turns toward a right side or a left side with respect to the vertical direction. That is, while the main body handle 42 provided in the main body 10 according to the first and second embodiments described above has to be made to face downward in order to insert the puncturing unit 30 into the nasal cavities, the main body handle 542 provided in the main body 510 of the present embodiment has to be made to face rightward or leftward at an ultrasonic portion according to whether a surgical procedure is performed on a right maxillary sinus 100 or a left maxillary sinus 100 in order to insert the puncturing unit 30 into the nasal cavities. Therefore, as illustrated in FIG. 45, the main body handle 542 is provided to face sideward when viewed from the bottom.

The second surgical procedure tube 511 may be configured to include the probing tube 511a configured to allow the posterior fontanelle 109 to be detected, the marking/puncturing tube 511b configured to allow a position to be punctured to be marked on the mucosa 108 at a portion expected to be the posterior fontanelle 109 in the middle meatus 103 and then allow the maxillary sinus 100 to be punctured, and the liquid tube 511c configured to allow a liquid to be injected into or discharged from a liquid structure 5222, which will be described below.

The second surgical procedure tube 511 may be provided in a lengthy form to be inserted into the middle meatus 103. Here, the probing tube 511a, the marking/puncturing tube 511b, and the liquid tube 511c which constitute the second surgical procedure tube 511 may be integrally manufactured to be parallel to each other in the longitudinal direction and may be connected to the ultrasonic probe 522, which will be described below, that is provided at the front end of the second surgical procedure tube 511.

In the present embodiment, as an example, the second surgical procedure tube 511 will be described below using the case in which the second surgical procedure tube 511 is manufactured as a model which does not include a configuration such as the irrigation tube 60 according to the first embodiment or the second embodiment described above. However, of course the irrigation tube 60 which is the same as or similar with that according to the first embodiment or the second embodiment may be included in the second surgical procedure tube 511, and thus, the maxillary sinus puncturing apparatus 3 may be manufactured as an integral type model in which the ultrasonic probing unit 520 and the marking/puncturing unit 530 are provided together in the main body 510.

The probing tube 511a may be provided in a lengthy form to be inserted into the middle meatus 103. The probing tube 511a is a tube provided to detect the posterior fontanelle 109 using ultrasonic waves. The ultrasonic probe 522 configured to detect the posterior fontanelle 109 may be connected to a front end of the probing tube 511a, an ultrasonic main body 521 configured to generate an electrical signal for generating ultrasonic waves may be connected to a rear end of the probing tube 511a, and a coaxial lead wire 523 configured to transmit the electrical signal may be disposed inside the probing tube 511a. Here, a front end of the coaxial lead wire 523 may be connected to the ultrasonic probe 522, and a rear end thereof may be connected to the ultrasonic main body 521 which will be described below. The ultrasonic main body 521 may be provided at a rear portion of the main body 510, and the probing tube 511a may have a structure connected from the ultrasonic probe 522 to the rear portion of the main body 510.

The marking/puncturing tube 511b may be provided in a lengthy form to be inserted into the middle meatus 103 together with the probing tube 511a and may be disposed to be vertically parallel to the probing tube 511a. The marking/puncturing tube 511b is disposed to be vertically adjacent to the probing tube 511a since nasal cavities are sufficient in the vertical direction but are limited in the horizontal direction.

The marking/puncturing tube 511b is a tube provided to mark a position to be punctured on the mucosa 108 at a portion expected to be the posterior fontanelle 109 in the middle meatus 103 and then puncture the maxillary sinus 100. The marking/puncturing tube 511b may have a structure connected from the ultrasonic probe 522 to the main body 510, an outlet portion 512 through which a marking/puncturing needle 531 moves in and out of the marking/puncturing tube 511b may be formed at a portion of the ultrasonic probe 522, and an inlet portion 513 through which the marking/puncturing needle 531 is inserted into the marking/puncturing tube 511b may be formed at a portion of the main body 510. Here, the outlet portion 512 of the marking/puncturing tube 511b may be formed so that the marking/puncturing tube 511b horizontally extends from the main body 510 to an inner portion of the ultrasonic probe 522 and then is obliquely penetrated to facilitate protruding of the marking/puncturing needle 531 instead of being vertically penetrated toward the liquid structure 5222.

The marking/puncturing needle 531 configured to mark and puncture the posterior fontanelle 109 and a cautery wire 532 configured to transmit electricity to the marking/puncturing needle 531 may be disposed inside the marking/puncturing tube 511b. Here, by moving back and forth inside the marking/puncturing tube 511b, the cautery wire 532 may allow the marking/puncturing needle 531 to protrude from the outlet portion 512 which forms the front end of the marking/puncturing tube 511b.

The marking/puncturing needle 531 disposed at the front end of the marking/puncturing tube 511b electrocauterizes the posterior fontanelle 109 by using electricity in order to leave a mark on the posterior fontanelle 109 and puncture the posterior fontanelle 109. Here, since heat is generated, the outlet portion 512 forming the front end of the marking/puncturing tube 511b may be insulated in order to be protected from heat. A method of insulating the front end of the marking/puncturing tube 511b is not particularly limited.

Since the outlet portion 512 of the marking/puncturing tube 511b is formed to be inclined as described above, the marking/puncturing needle 531 punctures the posterior fontanelle 109 while protruding obliquely instead of protruding at a right angle. In consideration of this, it may be preferable to mark a puncturing start point with the plus mark (+) on a probing monitor 527 during the actual surgical procedure, wherein the puncturing start point is marked on an edge of the posterior fontanelle 109 instead of being marked on the center thereof. Here, the plus mark (+) indicates a site where the tip of the marking/puncturing needle 531 is actually located when in contact with the mucosa of the posterior fontanelle 109.

The liquid tube 511c may be provided in a lengthy form to be inserted into the middle meatus 103 together with the probing tube 511a and the marking/puncturing tube 511b and may be disposed to be vertically parallel with the probing tube 511a and the marking/puncturing tube 511b. The liquid tube 511c is provided to be vertically adjacent to the probing tube 511a and the marking/puncturing tube 511b since nasal cavities are sufficient in the vertical direction but are limited in the horizontal direction.

The liquid tube 511c is a tube provided to inject or discharge a liquid into or from the liquid structure 5222 and may have a structure connected from the ultrasonic probe 522 to the main body 510. An outlet portion 514 of the liquid tube 511c may be connected to the liquid structure 5222 of the ultrasonic probe 522, and an inlet portion 515 of the liquid tube 511c may be connected to a liquid injection/discharge means 550 provided at a portion of the main body 510. The liquid injection/discharge means 550 may be provided at the inlet portion 515 of the liquid tube 511c in order to inject or discharge a liquid into or from the liquid structure 5222.

The ultrasonic probing unit 520 is a device configured to detect a punctured position of the posterior fontanelle 109 by using ultrasonic waves. The ultrasonic probing unit 520 may be provided in the main body 510 and detect the posterior fontanelle 109 where there are no bones 107 in a direction from the middle meatus 103 toward the maxillary sinus 100.

Here, a value detected by the ultrasonic probing unit 520 may be provided to the operator by being shown with an image or provided to the operator using various other method such as by being indicated with sound, vibration, or the like.

The ultrasonic probing unit 520 may include the ultrasonic main body 521, the ultrasonic probe 522, the coaxial lead wire 523, and the probing monitor 527.

The ultrasonic main body 521 is provided at the rear portion of the main body 510, is connected to a rear end of the coaxial lead wire 523, and generates an electrical signal for generating ultrasonic waves. Since the ultrasonic main body 521 has to have the size suitable for generating and analyzing an electrical signal, the ultrasonic main body 521 may be provided at the rear portion of the main body 510 which does not have to be inserted into the middle meatus 103. Of course the ultrasonic main body 521 may be provided at a position spaced apart from the main body 510 and connected to the main body 510 via a wire instead of being directly provided at the rear portion of the main body 510. Also, the ultrasonic main body 521 may be connected to the notification unit 50, which has been described above with reference to the first embodiment, via a wire or wirelessly and transmit a detected value to the notification unit 50.

The ultrasonic probe 522 may be provided at the front portion of the main body 510. In the second surgical procedure tube 511, as described above, the ultrasonic probe 522 may be disposed at the front end of the second surgical procedure tube 511. Specifically, the ultrasonic probe 522 may be provided at a front end of the probing tube 511a, which is an element of the second surgical procedure tube 511, and may be connected to the front end of the coaxial lead wire 523. Here, the ultrasonic probe 522 may detect the posterior fontanelle 109 in the middle meatus 103 by converting an electrical signal into ultrasonic waves and emitting the ultrasonic waves.

Here, an oval disk-shaped probe may be employed as the ultrasonic probe 522 so that the ultrasonic probe 522 may perform the marking/puncturing function or the liquid injection/discharge function in addition to its unique detecting function. The basic structure of the ultrasonic probe 522 may be the same as or similar to that of the ultrasonic probe 22 illustrated in FIG. 35.

Ultrasonic waves may be generated from one side surface of the oval disk-shaped ultrasonic probe 522, and the liquid structure 5222 may be further provided at the one side surface from which ultrasonic waves are generated. Also, the outlet portion 512 of the marking/puncturing tube 511b may be formed at the one side surface from which ultrasonic waves are generated. It may be preferable to form the outlet portion 512 of the marking/puncturing tube 511b at the central portion of the ultrasonic probe 522. In the present embodiment, the position at which the outlet portion 512 of the marking/puncturing tube 511b is formed is not particularly limited.

Since the oval disk-shaped ultrasonic probe 522 may detect the posterior fontanelle 109 by bringing the one side surface having a wide area into contact with the mucosa 108, the detection time may be shortened.

Generally, since propagation of ultrasonic waves is possible only when the probe is adhered to an object without an air layer therebetween, when the oval disk-shaped ultrasonic probe 522 is used at the portion of the posterior fontanelle 109 which is not flat, the propagation of ultrasonic waves becomes possible only when the shape of a surface of the probe changes according to the shape of the posterior fontanelle 109 so that the surface of the probe is adhered to the mucosa. In the present embodiment, the liquid structure 5222 allows the surface of the probe to be adhered to the mucosa.

The liquid structure 5222 may be provided at one side surface of the ultrasonic probe 522. Generally, the liquid structure 5222 is filled with a liquid, thereby allowing the detection using ultrasonic waves. In the present embodiment, a liquid may be injected into or discharged from the liquid structure 5222.

That is, when the liquid structure 5222 is always filled with a liquid, the thickness of the ultrasonic probe 522 is inevitably thick, and thus, it may not be easy to insert the ultrasonic probe 522 into the middle meatus 103. In order to address such an issue, the present embodiment adopts a structure in which a liquid is completely removed from the liquid structure 5222 until the ultrasonic probe 522 is inserted into the middle meatus 103 in a preparation step, and then, when the ultrasonic probe 522 is located at the posterior fontanelle 109, the liquid structure 5222 is filled with liquid.

The liquid structure 5222 may be connected to the outlet portion 514 of the liquid tube 511c. Liquid may be removed from and filled in the liquid structure 5222 by using the liquid injection/discharge means 550 provided at the inlet portion 515 of the liquid tube 511c.

The liquid injection/discharge means 550 may be configured to include a valve 551 provided inside the inlet portion 515 of the liquid tube 511c and a liquid supply unit 552 detachable from the inlet portion 515 of the liquid tube 511c.

The valve 551 may be a three-way valve, and the liquid supply unit 552 may be a syringe.

The liquid injection/discharge means 550 may remove liquid from the liquid structure 5222 or fill the liquid structure 5222 with liquid by using the liquid supply unit 552 and may maintain the amount of liquid in the liquid structure 5222 by using the valve 551. When an image shown on the probing monitor 527 is checked while injecting a liquid, if an image of the posterior fontanelle 109 is well-visible, no more liquid is injected into the liquid structure 5222 using the liquid supply unit 552.

The coaxial lead wire 523 may transmit an electrical signal from the ultrasonic main body 521 to the ultrasonic probe 522 and may be provided to pass through the inner portion of the probing tube 511a, which is an element of the second surgical procedure tube 511 described above.

One end of the coaxial lead wire 523 may be connected to the ultrasonic probe 522 at the front end of the probing tube 511a, and the other end of the coaxial lead wire 523 may be connected to the ultrasonic main body 521 at the rear portion of the main body 510.

The probing monitor 527 may be provided at a rear portion of the ultrasonic main body 521 and allows the posterior fontanelle 109, which is detected by the ultrasonic probe 522, to be viewed as an image during the surgical procedure.

The marking/puncturing unit 530 may be provided in the main body 510 and allow a position to be punctured to be marked on the mucosa 108 at a portion expected to be the posterior fontanelle 109 in the middle meatus 103 detected by the ultrasonic probing unit 520 and then allow the maxillary sinus 100 to be punctured.

The marking/puncturing unit 530 may include the marking/puncturing needle 531, the cautery wire 532, a marking/puncturing electrocauterizer main body 533, a cautery activation switch 534, a main body socket 536, and an emergency treatment hole 537.

The marking/puncturing needle 531 corresponds to a puncturing electrocauterizer that cauterizes the posterior fontanelle 109 using electricity. The marking/puncturing needle 531 may be manufactured as a bipolar type or the like and may also be manufactured in the form of a thermocauterizer.

The marking/puncturing needle 531 may be connected to the cautery wire 532 and may be disposed at the outlet portion 512 so as to mark a position to be punctured on the mucosa 108 and perform puncturing on the position.

The marking/puncturing needle 531 may be provided at a front end of the cautery wire 532, and a cautery wire socket 5321 may be provided at a rear end of the cautery wire 532. The cautery wire 532 may be connected to the marking/puncturing needle 531 and supply electricity thereto. The cautery wire socket 5321 may be connected to the main body socket 536 via an external wire. The main body socket 536 may be provided in the main body 510.

The cautery wire 532 may have a structure in which a wire connected to the marking/puncturing needle 531 is coated with an insulator. The cautery wire 32 may be in a thin form so as to be movable inside the marking/puncturing tube 511b.

By moving back and forth inside the marking/puncturing tube 511b due to a moving unit 541 of the operation unit 540, the cautery wire 532 may allow the marking/puncturing needle 531 to protrude from the outlet portion 512 which forms the front end of the marking/puncturing tube 511b.

The cautery wire 532 may be formed with a material that allows the cautery wire 532 to be bent along the inclined portion of the outlet portion 512 of the marking/puncturing tube 511b which is formed to be inclined. However, a portion of the cautery wire 532 which deviates from the front end of the marking/puncturing tube 511b may be formed with a material capable of firmly supporting the marking/puncturing needle 531.

A portion of the cautery wire 532 that extends from the inlet portion 513 of the marking/puncturing tube 511b to a moving block 5410 may be exposed to the outside without being protected by the marking/puncturing tube 511b. Since the exposed cautery wire 532 may be bent when the moving block 5410 moves forward, the cautery wire protection tube 322 according to the first embodiment or the cautery wire protection tube 322a according to the second embodiment, which has been described above, may be further provided in order to protect the exposed cautery wire 532.

The marking/puncturing electrocauterizer main body 533 may be provided inside the main body handle 542 of the main body 510 and may have a battery embedded therein. The marking/puncturing electrocauterizer main body 533 may be connected to the cautery wire 532 via a wire and allow electricity to be supplied to the marking/puncturing needle 531 via the cautery wire 532.

The main body socket 536 may be further provided between the marking/puncturing electrocauterizer main body 533 and the cautery wire 532 so as to connect or disconnect the wire. The main body socket 36 may be provided in the main body 510 and connected to the cautery wire socket 5321 via an external wire.

The cautery activation switch 534 may be provided in the main body 510. The cautery activation switch 534 may be connected to the marking/puncturing electrocauterizer main body 533 via a wire and activate a cauterizing function of the marking/puncturing electrocauterizer main body 533.

The cautery activation switch 534 may be configured to include a moving member 5341, a fixing member 5342, and a switch 5343. However, the cautery activation switch 534 is not limited thereto and may of course be realized in various other ways.

As illustrated in FIG. 49, a protrusion 5341a may be provided at a lower surface of the moving member 5341. The moving member 5341 is movable back and forth and turns on or off the cauterizing function.

The fixing member 5342 is fixed and installed at the main body 510 and serves to support the switch 5343.

The switch 5343 is installed at the fixing member 5342 and turns on or off the cauterizing function by rotating back and forth according to back and forth movement of the moving member 5341. The switch 5343 may be configured to turn on the cauterizing function only in the situation illustrated in FIG. 49B. Since the time taken for the switch 5343 to be turned on varies according to a length of the protrusion 5341a, the time required for turning on the switch 5343 may be obtained by varying the length of the protrusion 5341a.

The emergency treatment hole 537 may be formed in the ultrasonic probe 522 so that, when the marking/puncturing needle 531 of the marking/puncturing unit 530 for marking or puncturing is locked to an inner wall of the ultrasonic probe 522 forming the outlet portion 512 of the marking/puncturing tube 511b and thus is unable to smoothly move forward, for emergency treatment thereof, a tool such as a wire is inserted through the emergency treatment hole 537 so as to push the marking/puncturing needle 531 and change a direction thereof.

The emergency treatment hole 537 may be formed in the other side surface of the ultrasonic probe 522, which is a surface opposite to the liquid structure 5222. The emergency treatment hole 537 may be formed to pass through the outlet portion 512 of the marking/puncturing tube 511b from the other side surface of the ultrasonic probe 522.

The operation unit 540 may be provided in the main body 510 and may move the marking/puncturing unit 530, which is an element of the second surgical procedure tube 511, toward the posterior fontanelle 109.

The operation unit 540 is a configuration that allows the marking/puncturing needle 531 of the marking/puncturing unit 530 to come into contact with the posterior fontanelle 109 so that marking and puncturing of the posterior fontanelle 109 are realized. Specifically, the operation unit 540 may allow the cautery wire 532 to move back and forth inside the marking/puncturing tube 511b.

The operation unit 540 may include the moving unit 541, the main body handle 542, and a moving block position adjuster 543.

The moving unit 541 may move the cautery wire 532 along the marking/puncturing tube 511b. Particularly, the moving unit 541 may move the cautery wire 532 forward along the marking/puncturing tube 511b. When the cautery wire 532 moves forward, the marking/puncturing needle 531 connected to the front end of the cautery wire 532 may come into contact with the posterior fontanelle 109.

The moving unit 541 may include the moving block 5410, a rail 5412, and a trigger 5413.

The moving block 5410 may move while the cautery wire 532 is fixed and allow the cautery wire 532 to move along the inner portion of the marking/puncturing tube 511b. Of course the configuration of the moving block 5410 may be the same as or similar to that of the moving block 410 according to the first embodiment or the second embodiment described above.

The rail 5412 may be provided in the main body 510 and may guide movement of the moving block 5410. The rail 5412 may be provided in a T-shape, and a rail groove (not marked) into which the rail 5412 is inserted may be provided in the moving block 5410. In this way, the moving block 5410 may move back and forth while being fitted into the rail 5412 and not deviating therefrom.

Here, a roller or a sliding surface that facilitates movement of the moving block 5410 may be provided at the rail 5412, and the moving block 5410 may remain spaced apart from an upper surface of the main body 510 while moving along an upper surface of the rail 5412, so that the movement of the moving block 5410 is prevented from being restricted due to friction.

The rail 5412 may have a form that horizontally extends as much as a distance by which the cautery wire 532 has to move for the marking/puncturing needle 531 to come into contact with the posterior fontanelle 109. However, a step (not illustrated) configured to prevent deviation of the moving block 5410 may be provided on at least one of a front end and a rear end of the rail 5412. Of course the step may be provided on the upper surface of the rail 5412 and/or the upper surface of the main body 510.

The trigger 5413 may be provided in the main body 510. The trigger 5413 moves the moving block 5410 on the rail 5412 in accordance with the operator's manipulation. The trigger 5413 may be connected to the moving block 5410 via a wire 5414, and the wire 5414 may be wound around the wire roller 5415 provided at a front portion of the trigger 5413. When the trigger 5413 is moved backward by the operator's middle finger or index finger, the wire 5414 may be pulled as the wire roller 5415 is rotated, and thus the moving block 5410 may move forward.

Also, the trigger 5413 may be formed in a plate-shaped structure which is movable back and forth along the main body 510. Since the trigger 5413 is formed to have the plate-shaped structure, the trigger 5413 may interfere with the main body handle 542 when the trigger 5413 moves backward. In order to prevent the interference, an accommodating groove 5421 in which a rear end of the trigger 5413 may be accommodated may be formed in a front portion of the main body handle 542.

The main body handle 542 may be provided at a portion of the main body 510 in the form of a handle of a pistol so that the operator may easily grip the main body 510. When the trigger 5413 has the plate-shaped structure, the accommodating groove 5421 capable of accommodating the rear end of the trigger 5413 may be provided in the main body handle 542.

As compared with the main body handle 42 provided in the main body 10 according to the first and second embodiments described above that has to be made to face downward in order to insert the puncturing unit 30 into the nasal cavities, the main body handle 542 has to be made to face rightward or leftward at an ultrasonic portion according to whether a surgical procedure is performed on a right maxillary sinus 100 or a left maxillary sinus 100, in order to insert the marking/puncturing unit 530 into the nasal cavities. Therefore, as illustrated in FIG. 45, the main body handle 542 is provided to face sideward when viewed from the bottom.

The moving block position adjuster 543 may be provided in the main body 510 and installed at a rear portion of the moving block 5410 to adjust a position of the moving block 5410. The moving block position adjuster 543 includes a fixed wall 5431, a spring 5432, and a preventive wall 5433.

The spring 5432 may be installed between the rear end of the moving block 5410 and the fixed wall 5431 which is installed to be spaced a predetermined distance apart from the moving block 5410.

The length of the cautery wire 532 may be slightly increased or decreased as the maxillary sinus puncturing apparatus 3 is used. The moving block position adjuster 543 is provided to compensate for the increased or decreased length and prevents the moving block 5410 from moving from a predetermined position.

To manage the predetermined position of the moving block 5410 more quantitatively, the preventive wall 5433, which is configured to prevent further rearward movement of the moving block 5410 even when the spring 5432 pulls the moving block 5410 from the rear of the moving block 5410, is provided. The preventive wall 5433 is fixed by a fixing member such as a screw. A few preventive walls with different thicknesses may be prepared for the preventive wall 5433, and, when the moving block 5410 needs to be placed more forward or backward due to a changed length of the cautery wire 532, the preventive wall 5433 may be replaced with a preventive wall corresponding to the changed length.

The present invention is not limited to the embodiments described above and may include a combination of the above-described embodiments or a combination of at least one of the above-described embodiments and a known art as still another embodiment.

As described above, in the present invention, the level of difficulty and danger of a surgical procedure can be lowered due to not puncturing the bones 107, and the posterior fontanelle 109 where there are no bones 107 can be easily detected using ultrasonic waves and punctured, thereby promoting the operator's convenience.

The present invention has been described above on the basis of the embodiments of the present invention. However, the embodiments are merely examples, and the present invention is not limited thereto. Those of ordinary skill in the art to which the present invention pertains should understand that various combinations, modifications, or applications, which have not been described with reference to the above embodiments, are possible within the scope not departing from essential technical details of the embodiments described herein. Therefore, technical details related to modifications and applications which are easily derivable from the embodiments of the present invention should be interpreted as falling within the scope of the present invention.

1. An apparatus for puncturing a maxillary sinus, the apparatus comprising:
a main body gripped by an operator;
a probing unit detachable from the main body and configured to detect a posterior fontanelle;
a surgical procedure tube which is provided in the main body and formed of an endoscope unit configured to monitor the posterior fontanelle, a puncturing unit configured to puncture the posterior fontanelle, and an irrigation tube configured to irrigate the maxillary sinus through a punctured portion of the posterior fontanelle; and
an operation unit provided in the main body and configured to operate the surgical procedure tube.

2. The apparatus of claim 1, wherein the surgical procedure tube comprises a sheath configured to accommodate the puncturing unit, the irrigation tube, and the endoscope unit, the sheath comprising:
a guide structure which forms a front end of the sheath and has a convex outer shape and a round inner shape;
a sheath space which provides a place where a front end of the puncturing unit is bendable and in which a space region is secured by the guide structure;
a sheath opening configured to provide a passage through which the front end of the puncturing unit guided by the guide structure passes;
a surgical procedure means insertion passage through which the puncturing unit and the irrigation tube are inserted and mounted; and
an endoscope insertion passage through which the endoscope unit is inserted and mounted.

3. The apparatus of claim 2, wherein the sheath further comprises a direction-changing protrusion which is provided at an upper portion of an inner peripheral surface of the surgical procedure means insertion passage and configured to guide the front end of the puncturing unit to be bent toward the sheath opening before coming into contact with a round surface of the guide structure when the puncturing unit moves forward in order to puncture the posterior fontanelle.

4. The apparatus of claim 2, wherein the sheath further comprises an emergency treatment hole which is provided at an upper portion of the guide structure so as to pass through the sheath space and configured to allow emergency treatment to be performed when the puncturing unit or the irrigation tube is locked to an inner wall of the guide structure.

5. The apparatus of claim 2, wherein the guide structure is configured as a fixed type or a movable type.

6. The apparatus of claim 5, wherein the movable guide structure is configured so that a front portion is thick as a whole, a middle portion is thin as a whole, and a rear portion is thin as the middle portion but has a lower end which is thick as the front portion.

7. The apparatus of claim 5, wherein a fixing key configured to restrict a forward movement is further provided in the movable guide structure.

8. The apparatus of claim 2, wherein the puncturing unit comprises:
a puncturing needle which is provided in the surgical procedure means insertion passage, forms the front end of the puncturing unit, and is configured to cauterize the posterior fontanelle using electricity and puncture the posterior fontanelle;
a cautery wire which is provided in the surgical procedure means insertion passage, is moved by the operation unit, and passes through the surgical procedure means insertion passage so as to supply electricity to the puncturing needle; and
a puncturing electrocauterizer main body which is provided in the main body and configured to supply electricity to the puncturing needle through the cautery wire.

9. The apparatus of claim 8, wherein the puncturing unit further comprises:
a cautery activation button which is provided in the main body and configured to activate a cauterizing function of the puncturing electrocauterizer main body;
a switch which is provided in the main body and configured to turn on or off the electricity for driving the puncturing electrocauterizer main body; and
a socket which is provided in the main body and provided between the puncturing electrocauterizer main body and the cautery wire.

10. The apparatus of claim 8, wherein the irrigation tube is provided to be fitted to the cautery wire and is installed in the posterior fontanelle punctured by the puncturing needle.

11. The apparatus of claim 8, wherein:
a locking protrusion configured to prevent the irrigation tube from moving backward when the cautery wire moves forward is further provided;
a groove with an open upper portion is formed in the locking protrusion;
a towline is further provided at an inlet, through which drugs are injected, of the irrigation tube; and
in a state in which the irrigation tube is inserted into the maxillary sinus, the towline moves the irrigation tube upward and forward through the groove of the locking protrusion so that the irrigation tube is fixed so as not to be pushed backward.

12. The apparatus of claim 2, wherein:
the surgical procedure means insertion passage extends to a predetermined length forward from a front end of the main body and is formed of a portion inserted into nasal cavities during a surgical procedure and a portion provided at an upper portion of the main body and exposed to the outside during the surgical procedure;
a passage diameter of the portion exposed to the outside is larger than a passage diameter of the portion inserted into the nasal cavities; and
an inlet portion is provided at a rear end of the portion exposed to the outside.

13. The apparatus of claim 12, wherein:
the puncturing unit further comprises a cautery wire protection tube which has one end fixed to the operation unit and extends to a boundary between the portion inserted into the nasal cavities and the portion exposed to the outside; and
the cautery wire protection tube is inserted and mounted on the portion exposed to the outside and is fixed by a fixing clip provided at the inlet portion.

14. The apparatus of claim 2, wherein the endoscope unit comprises:
an endoscope camera main body provided in the main body;
an endoscope camera monitor rotatably mounted on the endoscope camera main body;
an endoscope camera head provided in the main body and connected to the endoscope camera main body;
an endoscope head provided in the main body and connected to the endoscope camera head;
an endoscope mounted on an inner portion of the endoscope insertion passage and connected to the endoscope head;
an endoscope window connected to a front end of the endoscope and provided to be exposed to the outside at a front end of the endoscope insertion passage so as to secure a monitoring field-of-view; and
a small light-emitting diode (LED) light source device or light source cable configured to transmit light to the endoscope.

15. The apparatus of claim 14, wherein the endoscope camera main body is provided at a rear end of the main body, has a battery embedded therein, and is manufactured so that an upper end surface thereof is placed on a straight line with an upper end surface of the main body.

16. The apparatus of claim 1, wherein the probing unit is an ultrasonic probing unit configured to detect a punctured position of the posterior fontanelle using ultrasonic waves, the ultrasonic probing unit comprising:
an ultrasonic main body provided at a rear end of a probing tube;
an ultrasonic probe which is provided at a front end of the probing tube and has one side surface at which a liquid structure is provided; and
a coaxial lead wire provided inside the probing tube and configured to transmit an electrical signal from the ultrasonic main body to the ultrasonic probe.

17. The apparatus of claim 16, wherein the ultrasonic probing unit further comprises:
a probing unit handle provided at a front portion of the ultrasonic main body; and
a probing monitor provided at a rear portion of the ultrasonic main body.

18. The apparatus of claim 16, further comprising a marking unit configured to mark a position to be punctured on a mucosa of the posterior fontanelle, wherein the marking unit comprises:
a marking electrocauterizer main body provided between the ultrasonic main body and the probing tube;
a marking activation button configured to activate a marking function of the marking electrocauterizer main body;
a needle passage which is provided in the ultrasonic probe and has a structure open toward the liquid structure;
a wire which is provided inside the probing tube and extends from the marking electrocauterizer main body to the needle passage; and
a marking needle which is provided inside the needle passage and connected to the wire so as to receive electricity from the marking electrocauterizer main body, cauterize the mucosa of the posterior fontanelle, and mark the position to be punctured thereon.

19. The apparatus of claim 16, further comprising a marking unit configured to mark a position to be punctured on a mucosa of the posterior fontanelle, wherein the marking unit comprises:
a cylinder tube which is provided in the ultrasonic probe and has a structure open toward the liquid structure;
a fluid pipe which is provided in a longitudinal direction inside the probing tube and extends to the cylinder tube by passing through the ultrasonic probe;
a push-pull means which is connected to one end of the fluid pipe at an inner upper portion of the cylinder tube and configured to expand or contract due to a fluid injected or discharged through the fluid pipe;
a piston which is connected to the push-pull means at an inner lower portion of the cylinder tube and configured to move upward and downward due to an operation of the push-pull means; and
a marking needle which is connected to the piston at the inner lower portion of the cylinder tube and configured to, according to upward and downward movement of the piston, leave a scratch on the mucosa of the posterior fontanelle to mark a position to be punctured thereon.

20. The apparatus of claim 19, wherein:
a plurality of guide bars are provided at an inner lower surface of the cylinder tube to allow the piston to move upward and downward uniformly; and
a plurality of guide grooves corresponding to the plurality of guide bars are formed in the piston.

21. The apparatus of claim 16, further comprising a marking unit configured to mark a position to be punctured on a mucosa of the posterior fontanelle, wherein the marking unit comprises:
a cylinder tube which is provided in the ultrasonic probe and has a structure open toward the liquid structure;
a fluid pipe which is provided in a longitudinal direction outside the probing tube and extends to the cylinder tube by passing through the ultrasonic probe;
a piston which is accommodated in the cylinder tube and configured to move upward and downward due to a fluid injected or discharged through the fluid pipe;
a wire which is provided inside the fluid pipe and extends from an electricity supply source to the cylinder tube;
an airtight member which has one end connected to the cylinder tube and the other end connected to the piston and is configured to prevent the fluid injected into the fluid pipe from leaking to the outside; and
a tip which is provided at a lower end of the piston and configured to mark a position to be punctured on the mucosa of the posterior fontanelle using an electrocauterization method.

22. The apparatus of claim 21, wherein the tip marks the position to be punctured on the mucosa of the posterior fontanelle and then punctures using the electrocauterization method.

23. The apparatus of claim 21, further comprising a surgical procedure means insertion passage which is provided in the shape of a tunnel at an inner upper portion of the probing tube in the longitudinal direction in order to allow the puncturing unit and the irrigation tube to be mounted.

24. The apparatus of claim 1, wherein the probing unit is a light probing unit configured to detect a punctured position of the posterior fontanelle using light, the light probing unit comprising:
a light probing main body;
a light emitting unit formed of a first probing tube, which is connected to the light probing main body, and a first light probe, which is provided at a front end of the first probing tube and configured to receive an electrical signal from the light probing main body and emit light; and
a light receiving unit formed of a second probing tube, which is connected to the light probing main body, and a second light probe, which is provided at a front end of the second probing tube and configured to sense light emitted from the first light probe and transmit the light to the light probing main body.

25. An apparatus for puncturing a maxillary sinus, the apparatus comprising:
a main body gripped by an operator;
a surgical procedure tube provided in the main body; and
an operation unit configured to operate the surgical procedure tube,
wherein the surgical procedure tube comprises:
a probing tube configured to allow a posterior fontanelle to be detected by an ultrasonic probing unit; and
a marking/puncturing tube configured to allow the maxillary sinus to be punctured after a position to be punctured is marked on a mucosa of the posterior fontanelle by a marking/puncturing unit.

26. The apparatus of claim 25, wherein the surgical procedure tube further comprises a liquid tube configured to allow a liquid to be injected into or discharged from a liquid structure provided at one side surface of an ultrasonic probe of the ultrasonic probing unit.

27. The apparatus of claim 26, wherein each of the probing tube, the marking/puncturing tube, and the liquid tube is provided in a lengthy form to be inserted into a middle meatus and is disposed to be parallel to one another.

28. The apparatus of claim 26, wherein the liquid tube has a structure connected from the ultrasonic probe to the main body, an outlet portion of the liquid tube is connected to the liquid structure, and an inlet portion of the liquid tube is connected to a liquid injection/discharge means provided at a portion of the main body.

29. The apparatus of claim 28, wherein the liquid injection/discharge means comprises:
a valve provided inside the inlet portion of the liquid tube; and
a liquid supply unit detachable from the inlet portion of the liquid tube.

30. The apparatus of claim 25, wherein the ultrasonic probing unit comprises:
an ultrasonic main body which is provided at a rear end of the probing tube at a rear portion of the main body and configured to generate an electrical signal for generating ultrasonic waves;
an ultrasonic probe which is provided at a front end of the probing tube and configured to detect the posterior fontanelle;
a liquid structure provided at one side surface of the ultrasonic probe;
a coaxial lead wire which is provided inside the probing tube to pass therethrough and configured to transmit an electrical signal from the ultrasonic main body to the ultrasonic probe; and
a probing monitor provided at a rear portion of the ultrasonic main body.

31. The apparatus of claim 30, wherein the probing tube has a structure connected from the ultrasonic probe to the rear portion of the main body, the ultrasonic probe is connected to a front end of the probing tube, the ultrasonic main body is connected to a rear end of the probing tube, and the coaxial lead wire configured to transmit an electrical signal to the ultrasonic probe is disposed inside the probing tube.

32. The apparatus of claim 25, wherein the marking/puncturing unit comprises:
a marking/puncturing needle which is disposed at an outlet portion of the marking/puncturing tube and configured to cauterize the posterior fontanelle, mark a position to be punctured on a mucosa of the posterior fontanelle, and then puncture the maxillary sinus;
a cautery wire which is provided inside the marking/puncturing tube and moved by the operation unit and is configured to pass through the marking/puncturing tube to supply electricity to the marking/puncturing needle; and
a marking/puncturing electrocauterizer main body which is provided in the main body and configured to supply electricity to the marking/puncturing needle through the cautery wire.

33. The apparatus of claim 32, wherein the marking/puncturing unit further comprises:
a cautery activation switch which is provided in the main body and configured to activate a cauterizing function of the marking/puncturing electrocauterizer main body; and
a main body socket which is provided in the main body and provided between the marking/puncturing electrocauterizer main body and the cautery wire.

34. The apparatus of claim 32, wherein the marking/puncturing unit further comprises an emergency treatment hole which is formed at the other side surface of the ultrasonic probe, which is a surface opposite to the liquid structure provided at the one side surface of the ultrasonic probe of the ultrasonic probing unit, and formed to pass through the outlet portion of the marking/puncturing tube from the other side surface of the ultrasonic probe.

35. The apparatus of claim 34, wherein the marking/puncturing tube has a structure connected from the ultrasonic probe to the main body, the outlet portion through which the marking/puncturing needle moves in and out of the marking/puncturing tube is formed at a portion of the ultrasonic probe, an inlet portion through which the marking/puncturing needle is inserted into the marking/puncturing tube is formed at a portion of the main body, and the cautery wire configured to transmit electricity to the marking/puncturing needle is disposed inside the marking/puncturing tube.
